(19) United States
(12) Patent Application Publication
(10) Pub. No.: US 2025/0375465 A1
(43) Pub. Date: Dec. 11, 2025

(54) METHODS FOR AFFECTING TISSUE-RESIDENT T CELLS

(71) Applicant: THE TRUSTEES OF THE UNIVERSITY OF PENNSYLVANIA, Philadelphia, PA (US)

(72) Inventor: Christoph T. Ellebrecht, Philadelphia, PA (US)

(21) Appl. No.: 19/193,495

(22) Filed: Apr. 29, 2025

Related U.S. Application Data
(60) Provisional application No. 63/640,047, filed on Apr. 29, 2024.

(51) Int. Cl. A61K 31/7105; A61K 9/00; A61K 31/395; A61K 38/17; A61P 17/00; C07K 16/28; C12N 5/0783; C12N 5/10; C12N 9/22; C12N 15/113; C12Q 1/68

(52) U.S. Cl. CPC A61K 31/7105; A61K 9/0014; A61K 31/395; A61K 38/1709; A61P 17/00; C07K 16/2896; C12N 5/0636; C12N 5/10; C12N 9/226; C12N 15/113; C12Q 1/68; C12N 2310/20; C12N 2510/00; C12Q 2600/136

(57) ABSTRACT

Described herein are methods for reducing tissue resident memory T cells (TRM) in the skin, as well as a method for treating, ameliorating, and/or preventing autoimmune and inflammatory diseases or disorders in the skin. These methods include downregulating the expression of GSPT1 in the tissue resident T cells. Also described are methods for screening therapeutic targets for regulating T cell functions.

### CROSS REFERENCE TO RELATED APPLICATION

The present application is entitled to priority under 35 U.S.C. § 119 (e) to U.S. Provisional Patent Application No. 63/640,047, filed Apr. 29, 2024, which is hereby incorporated by reference in its entirety herein.

### SEQUENCE LISTING

The XML file named “046483-7456US1 (03892) Sequence Listing.xml” created on Apr. 27, 2025, comprising 9,054 bytes, is hereby incorporated by reference in its entirety.

### BACKGROUND

The skin is the most commonly affected organ in autoimmune and inflammatory diseases and disorders. T cell-mediated inflammatory and autoimmune skin diseases in particular, such as psoriasis, lichen planus, vitiligo, alopecia areata, and graft-versus-host disease, affect millions of people worldwide, leading to over $100 billion in annual healthcare costs in the US alone. Similarly, alopecia areata affects about 700,000 people, lichen planus affects about 3,000,000 people, lupus affects about 1,500,000 people, psoriasis affects about 7,500,000 people, and vitiligo affects about 2,800,000 people.

Despite decades of therapeutic advances, these devastating conditions continue to follow a frustratingly predictable pattern: treatment, temporary and mostly incomplete improvement, and inevitable relapse upon treatment reduction or cessation. As a result, existing treatments rarely achieve durable results, often require long term or even life-long administration, and are considered as long-term management strategies rather than as a cure.

Therefore, there is a need for methods of treating, ameliorating and/or preventing autoimmune and inflammatory diseases or disorders in the skin, especially those that can achieve long-term effectiveness. The present invention addresses this need.

### SUMMARY OF THE INVENTION

As described herein, the present disclosure relates to methods and compositions useful for reducing the presence of tissue-resident memory T cells (TRM) in the skin, as well as methods for treating, ameliorating, and/or preventing autoimmune and inflammatory diseases or disorders in the skin by reducing or inhibiting the pathologic function of skin TRM. In certain embodiments, the methods include downregulating the expression of GSPT1 in T cells, including TRM. Also described are methods for screening therapeutic targets for regulating T cell functions.

In one aspect, provided is a method of reducing skin-homing T cells or tissue-resident memory T cells (TRM) in a skin tissue, comprising contacting the skin-homing T cells or TRM with an effective amount of a composition that downregulates an expression level and/or an activity of GSPT1.

In certain embodiments, the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

In certain embodiments, the composition is delivered by a T cell-specific delivery method.

In certain embodiments, the composition comprises a lipid nanoparticle (LNP) that is conjugated to an antibody targeting a T cell specific surface antigen.

In certain embodiments, the T cell specific surface antigen is CD5.

In certain embodiments, the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

In certain embodiments, the composition comprises a molecular degrader that links GSPT1 to a ubiquitin ligase.

In certain embodiments, the composition comprises a small molecule inhibitor of GSPT1, and wherein the small molecule inhibitor of GSPT1 comprises at least one selected form the group consisting of SP-3204, CDG0501,

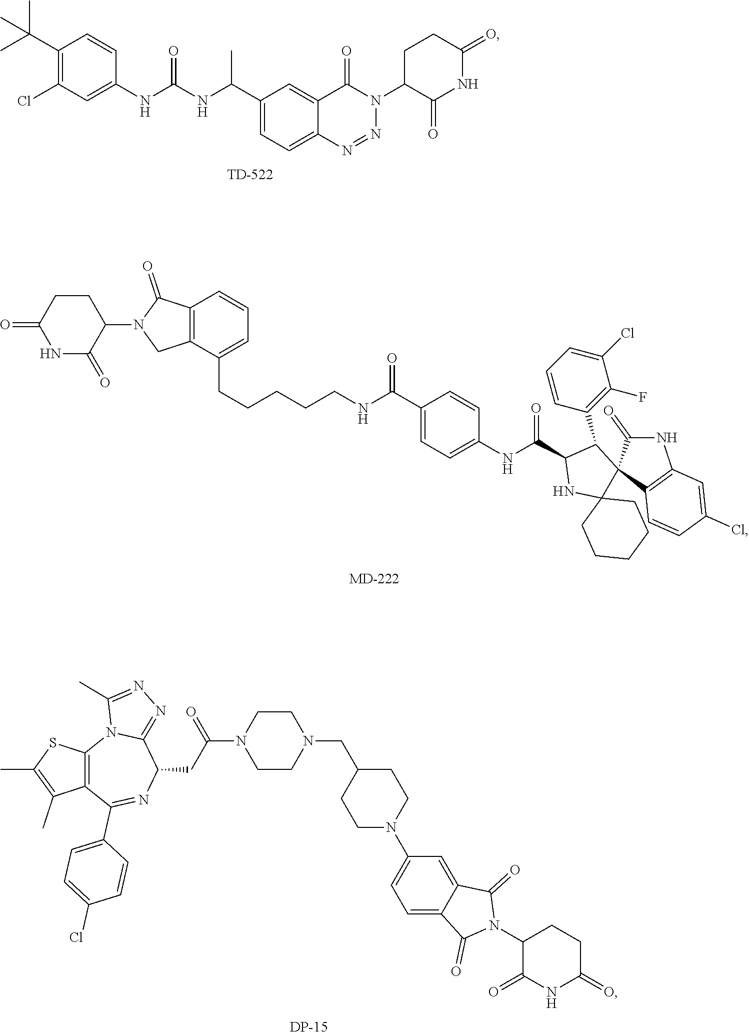 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; "DP-15"]

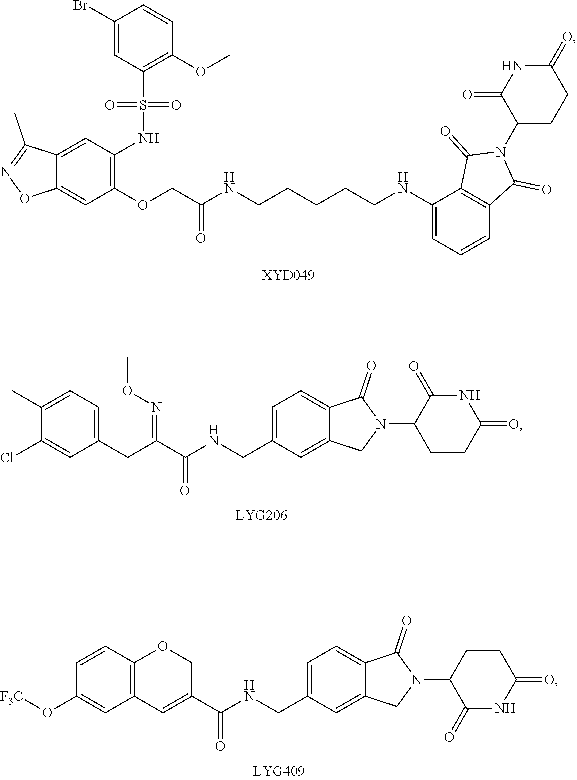

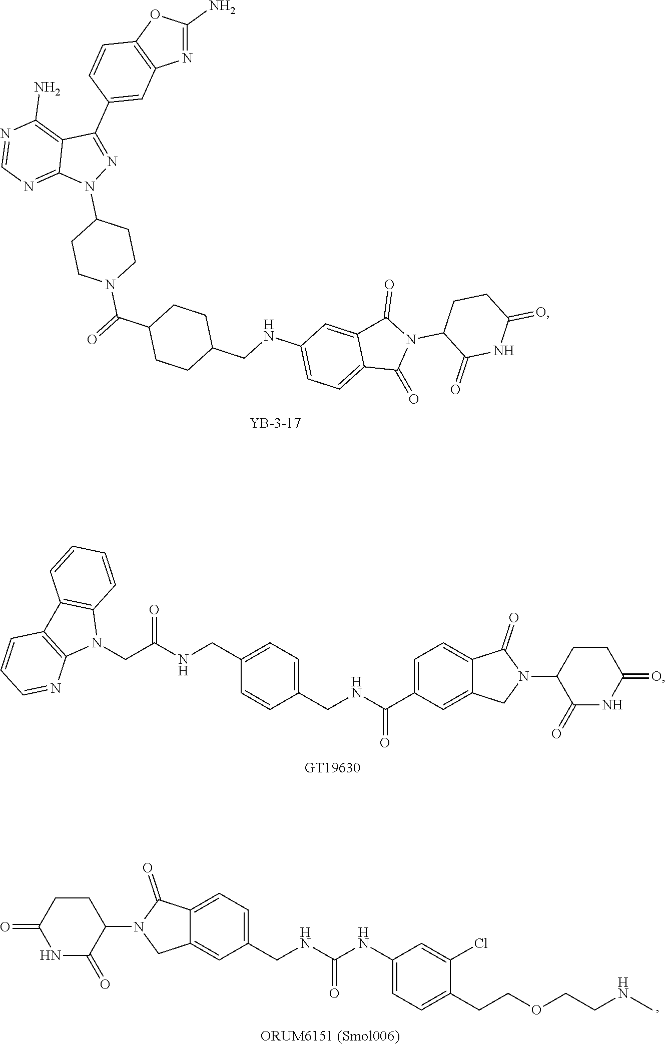 [SMILES: Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; "YB-3-17" ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl]

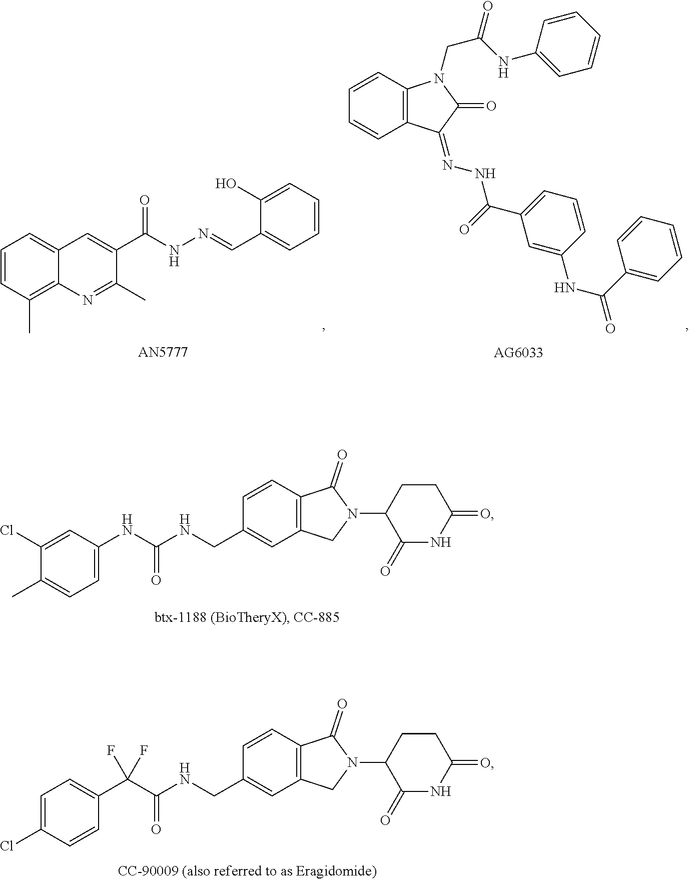 [SMILES: Cc1nc2c(C)cccc2cc1C(=O)N/N=C/c1ccccc1O ; O=C(CN1C(=O)/C(=N\NC(=O)c2cccc(NC(=O)c3ccccc3)c2)c2ccccc21)Nc1ccccc1 ; Cc1ccc(NC(=O)NCc2ccc3c(c2)CN(C2CCC(=O)NC2=O)C3=O)cc1Cl ; O=C1CCC(N2Cc3cc(CNC(=O)C(F)(F)c4ccc(Cl)cc4)ccc3C2=O)C(=O)N1]

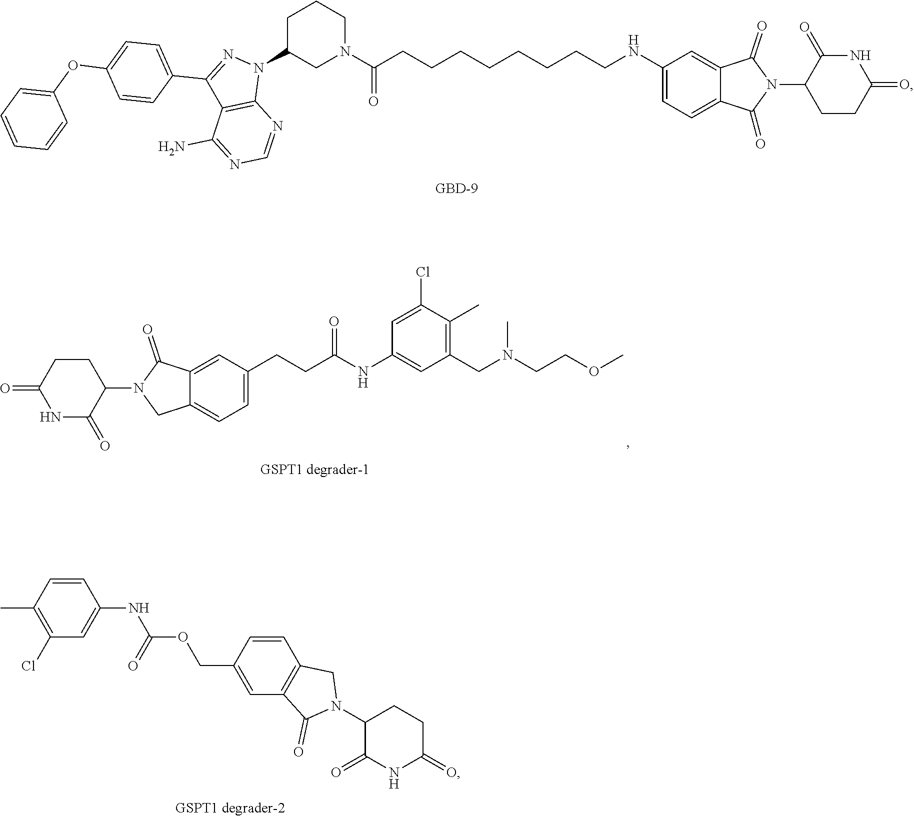

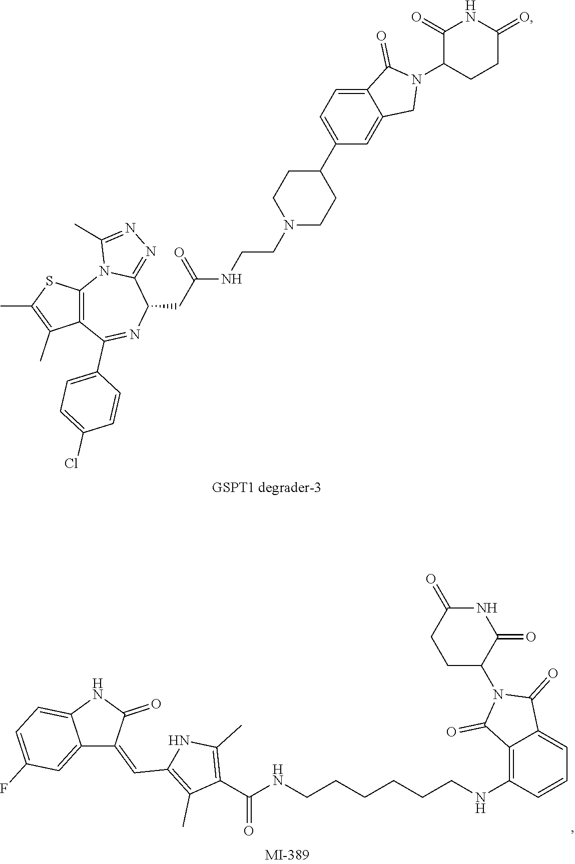 [SMILES: Cc1sc2c(c1C)C(c1ccc(Cl)cc1)=N[C@@H](CC(=O)NCCN1CCC(c3ccc4c(c3)CN(C3CCC(=O)NC3=O)C4=O)CC1)c1nnc(C)n1-2 ; Cc1[nH]c(/C=C2\C(=O)Nc3ccc(F)cc32)c(C)c1C(=O)NCCCCCCNc1cccc2c1C(=O)N(C1CCC(=O)NC1=O)C2=O]

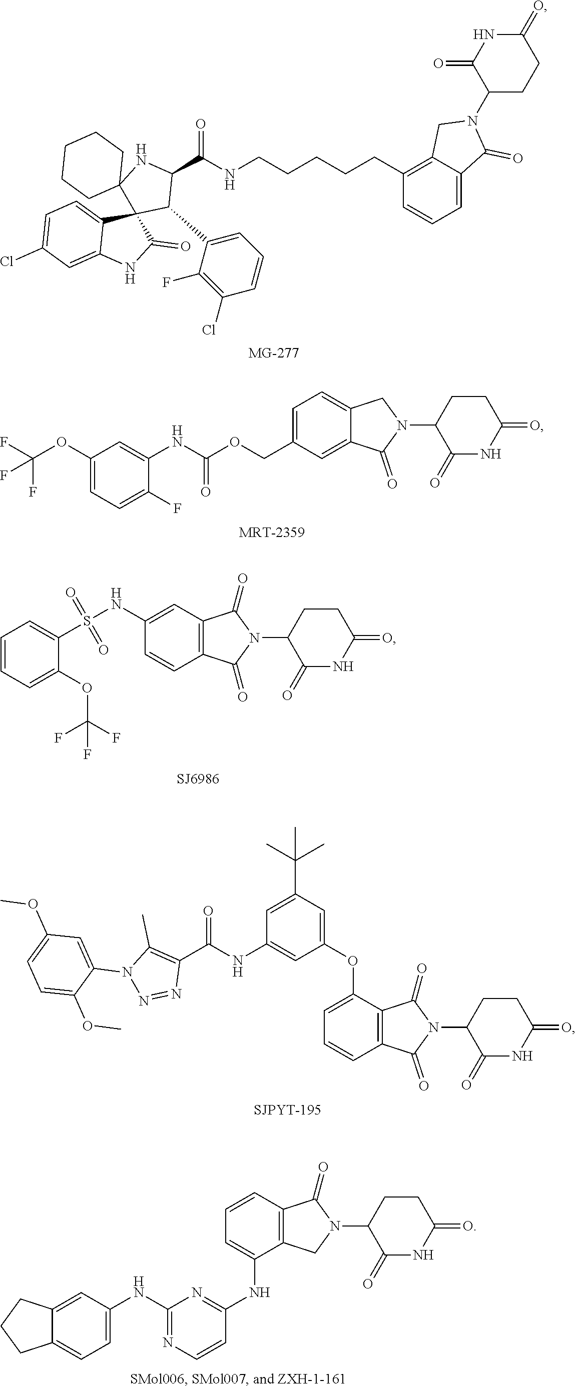 [SMILES: O=C1CCC(N2Cc3c(CCCCCNC(=O)[C@@H]4NC5(CCCCC5)[C@@]5(C(=O)Nc6cc(Cl)ccc65)[C@H]4c4cccc(Cl)c4F)cccc3C2=O)C(=O)N1 ; O=C1CCC(N2Cc3ccc(COC(=O)Nc4cc(OC(F)(F)F)ccc4F)cc3C2=O)C(=O)N1 ; O=C1CCC(N2C(=O)c3ccc(NS(=O)(=O)c4ccccc4OC(F)(F)F)cc3C2=O)C(=O)N1 ; COc1ccc(OC)c(-n2nnc(C(=O)Nc3cc(Oc4cccc5c4C(=O)N(C4CCC(=O)NC4=O)C5=O)cc(C(C)(C)C)c3)c2C)c1 ; O=C1CCC(N2Cc3c(Nc4ccnc(Nc5ccc6c(c5)CCC6)n4)cccc3C2=O)C(=O)N1]

In certain embodiments, administering an effective amount of the composition causes apoptosis in the TRM cells in the skin tissue.

In certain embodiments, wherein at least one of the following applies:
(i) the skin tissue is an isolated skin tissue; or
(ii) the skin tissue is in a subject.

In certain embodiments, the skin tissue is in a subject, and wherein the subject is a mammal, optionally a human.

In another aspect, provided is a method of selectively depleting T cells in the skin, comprising:
contacting T cells in the skin with a composition that downregulates an expression level and/or an activity of GSPT1.

In certain embodiments, the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

In certain embodiments, the composition is delivered by a T cell-specific delivery method.

In certain embodiments, the composition comprises a lipid nanoparticle, wherein the lipid nanoparticle is conjugated to an antibody targeting a T cell specific surface antigen.

In certain embodiments, the T cell specific surface antigen is CD5.

In certain embodiments, the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

In certain embodiments, the composition comprises a molecular degrader that links GSPT1 to a ubiquitin ligase.

In certain embodiments, the composition comprises a small molecule inhibitor of GSPT1, and wherein the small molecule inhibitor of GSPT1 comprises at least one compound selected form the group consisting of SP-3204, CDG0501,

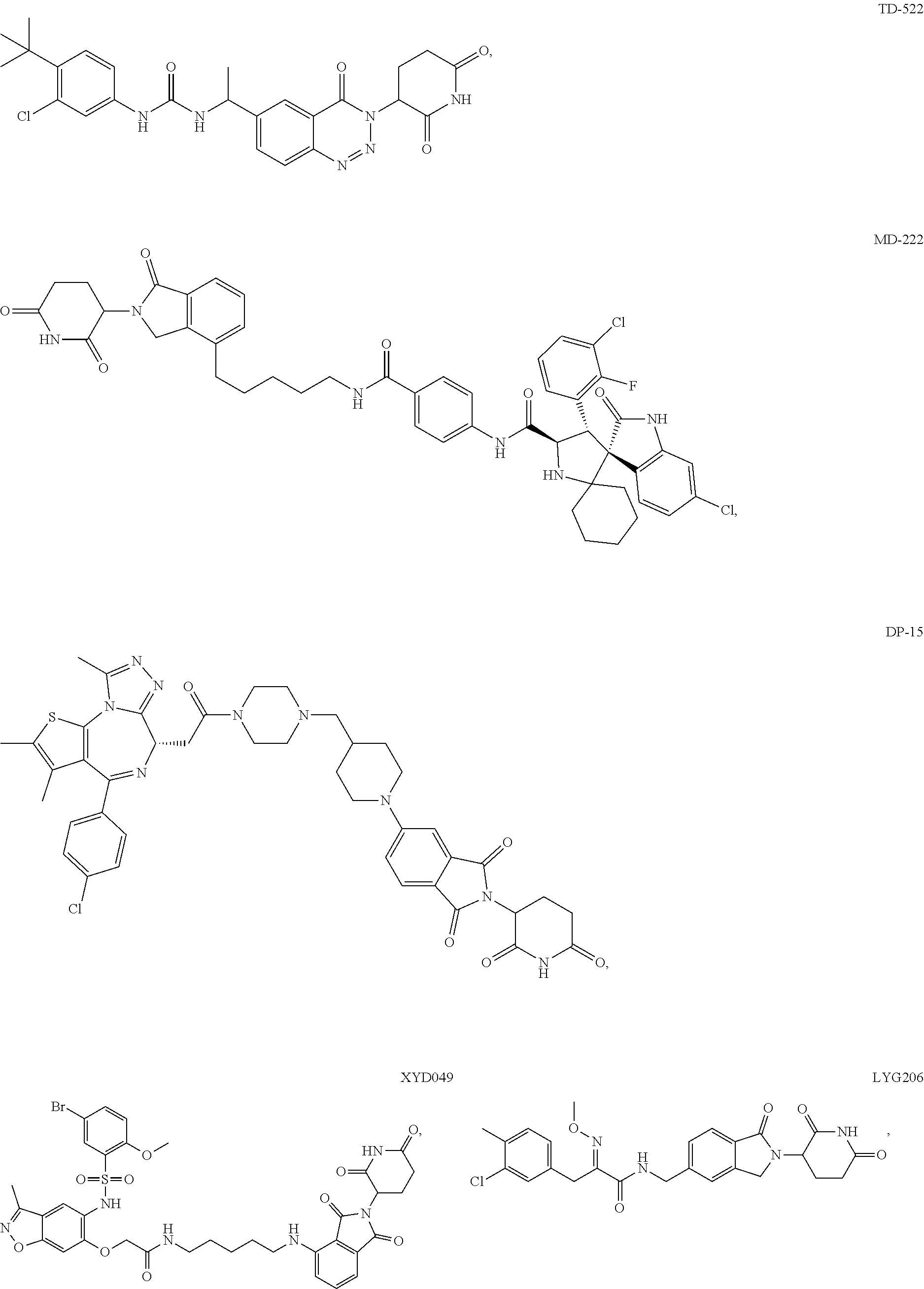 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O ; CO/N=C(\Cc1ccc(C)c(Cl)c1)C(=O)NCc1ccc2c(c1)CN(C1CCC(=O)NC1=O)C2=O]

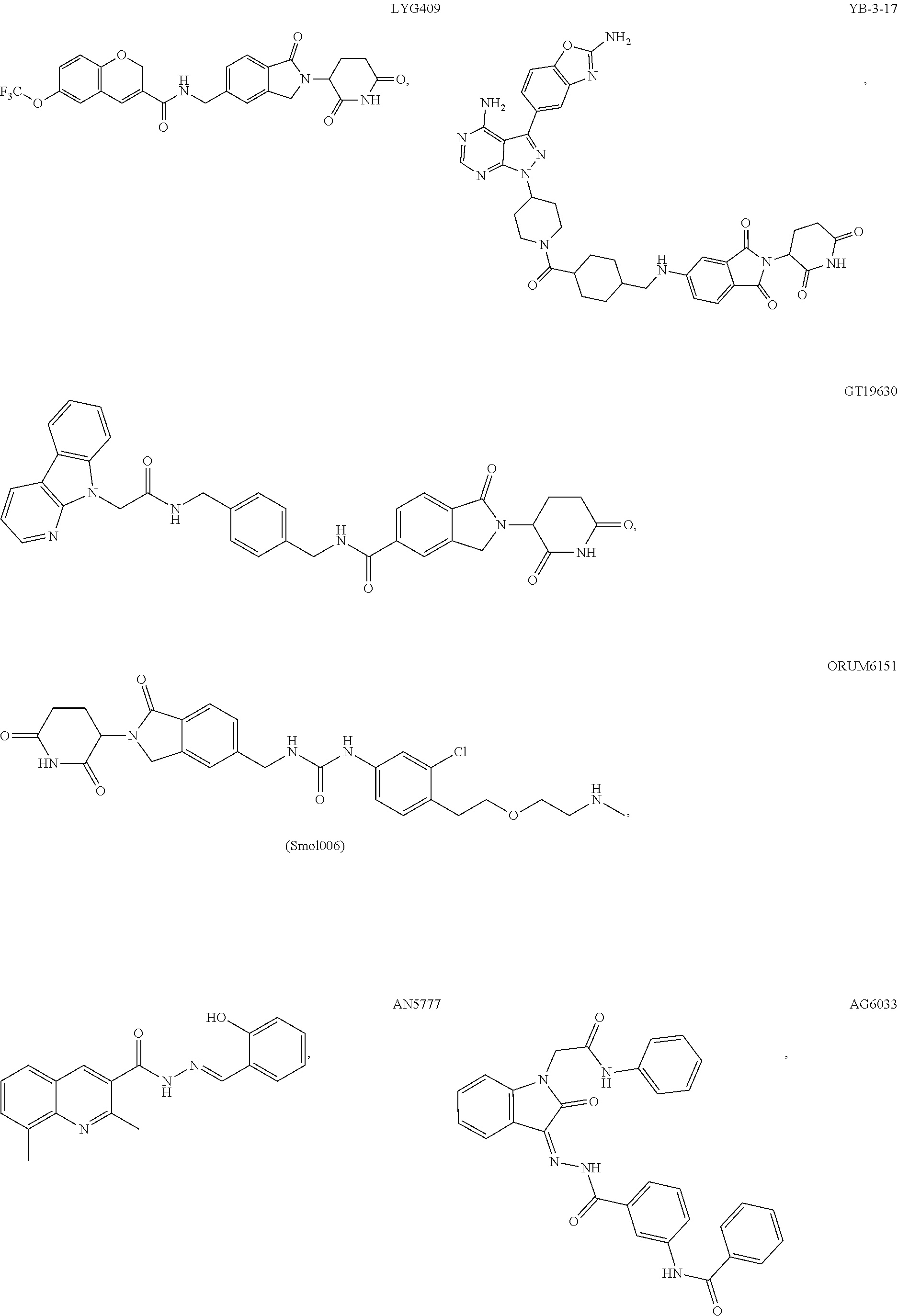 [SMILES: O=C1CCC(N2Cc3cc(CNC(=O)C4=Cc5cc(OC(F)(F)F)ccc5OC4)ccc3C2=O)C(=O)N1 ; Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl ; Cc1nc2c(C)cccc2cc1C(=O)N/N=C/c1ccccc1O ; O=C(CN1C(=O)/C(=N\NC(=O)c2cccc(NC(=O)c3ccccc3)c2)c2ccccc21)Nc1ccccc1]

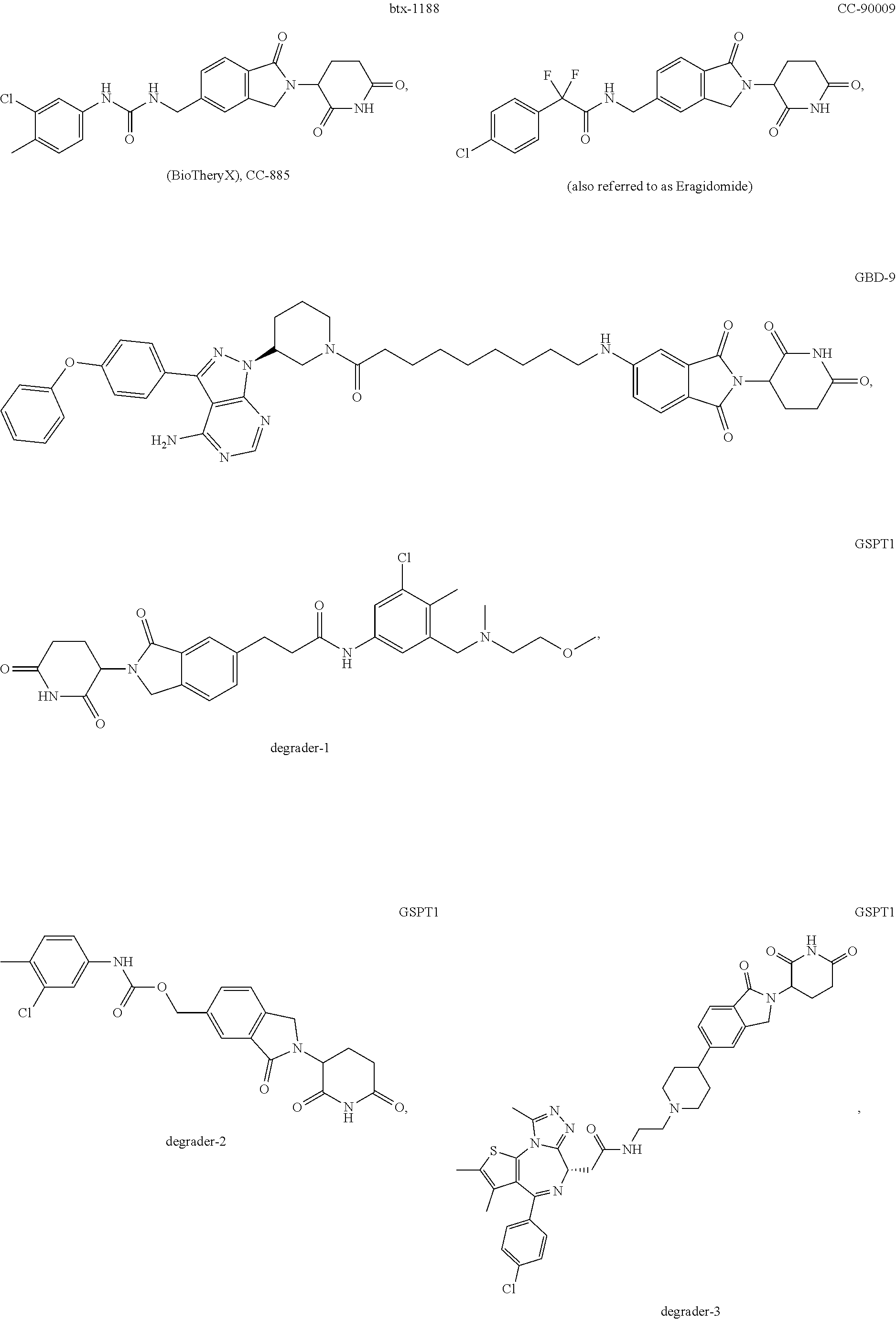

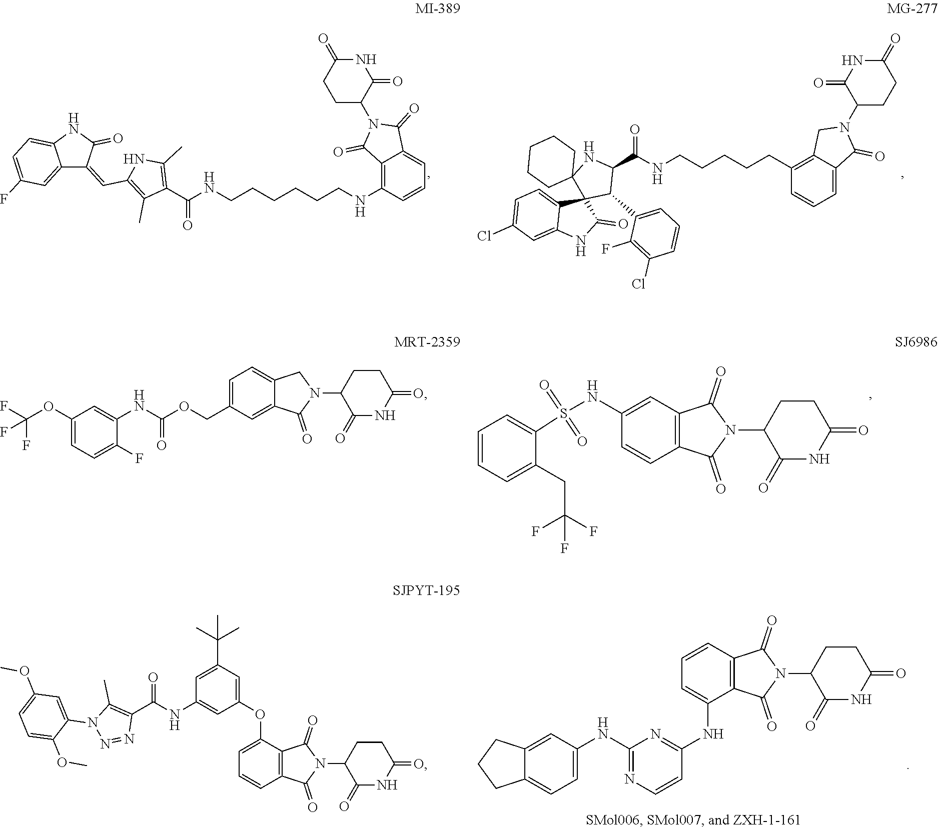

In certain embodiments, administering an effective amount of the compound causes apoptosis in the TRM cells in the skin tissue.

In certain embodiments, at least one of the following applies:
(i) the skin tissue is an isolated skin tissue; or
(ii) the skin tissue is in a subject.

In certain embodiments, the skin tissue is in a subject, and wherein the subject is a mammal, optionally a human.

In another aspect, provided is a method of treating, ameliorating and/or preventing an autoimmune or inflammatory disease or disorder in a skin in a subject in need thereof, comprising:
administering to the subject an effective amount of a composition that downregulates an expression level and/or an activity of GSPT1.

In certain embodiments, the autoimmune or inflammatory disease or disorder in the skin is alopecia areata, graft-versus-host disease (GVHD), lichen planus, lupus erythematosus, scleroderma (optionally localized forms such as morphea), psoriasis and related conditions such as psoriatic arthritis, Stevens-Johnson syndrome, urticarial vasculitis, vitiligo, atopic dermatitis (eczema), or bullous pemphigoid.

In certain embodiments, the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

In certain embodiments, the composition is delivered by a T cell-specific delivery method.

In certain embodiments, the compound further comprises a lipid nanoparticle, wherein the lipid nanoparticle is conjugated to an antibody targeting a T cell specific surface antigen.

In certain embodiments, the T cell specific surface antigen is CD5.

In certain embodiments, the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

In certain embodiments, the composition comprises a molecular degrader that links GSPT1 to a ubiquitin ligase.

In certain embodiments, the composition comprises a small molecule inhibitor of GSPT1, and wherein the small molecule inhibitor of GSPT1 comprises at least one selected form the group consisting of SP-3204, CDG0501,

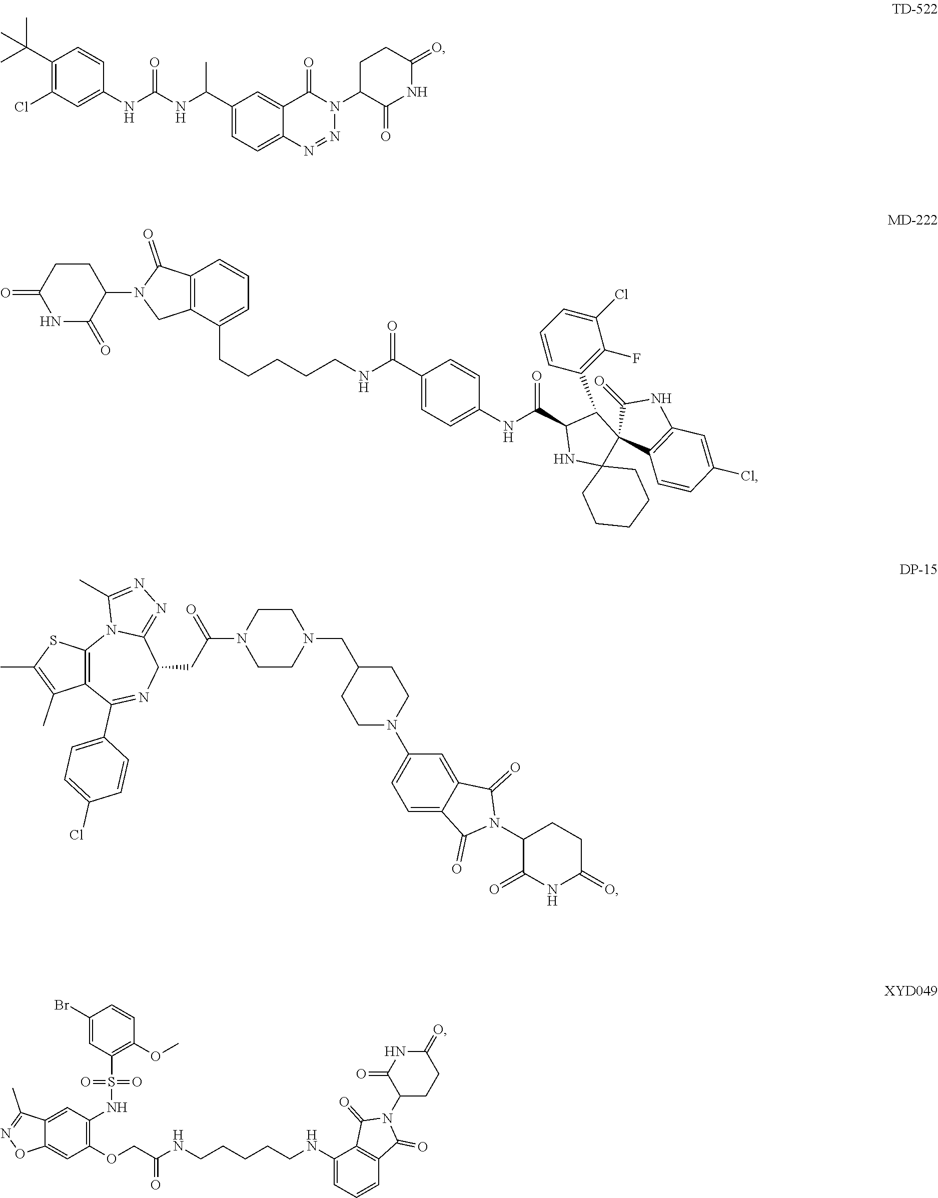 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O]

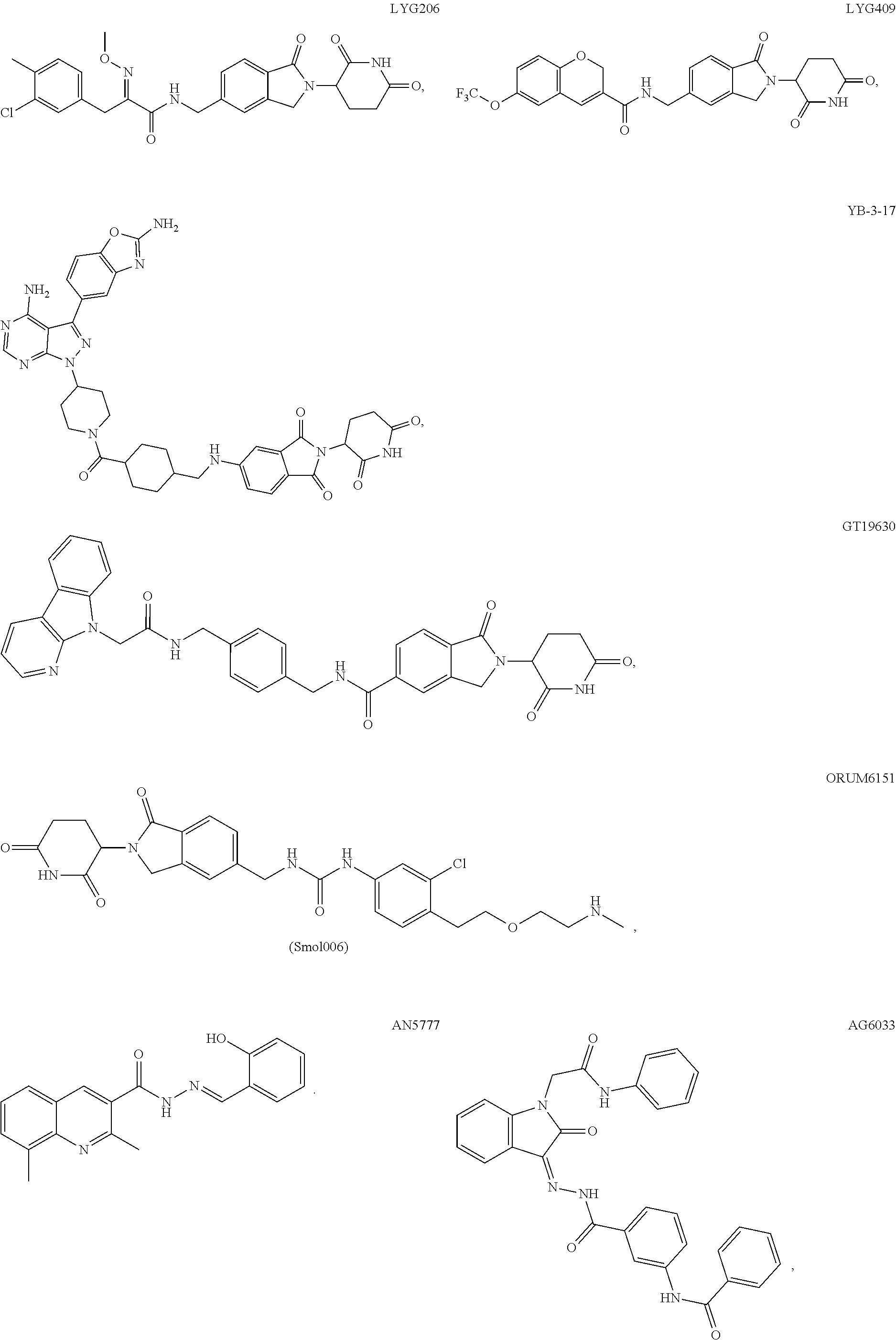

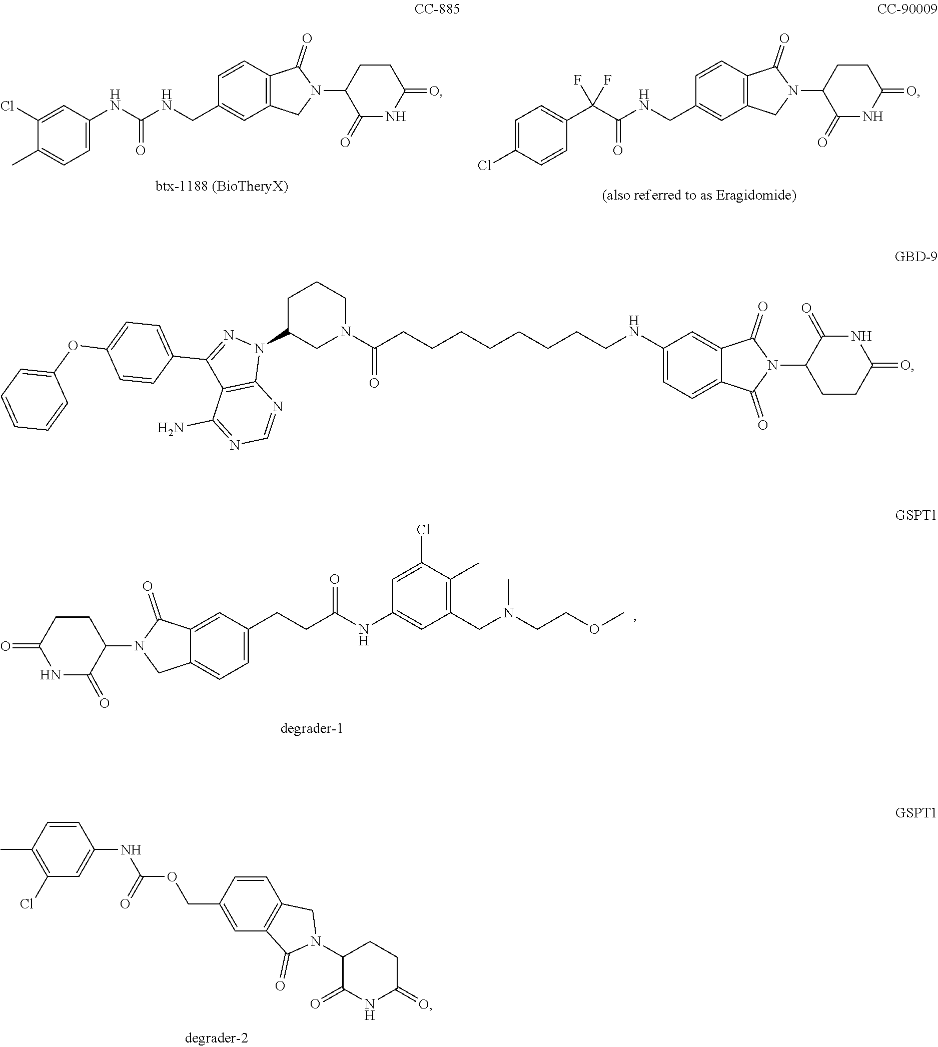

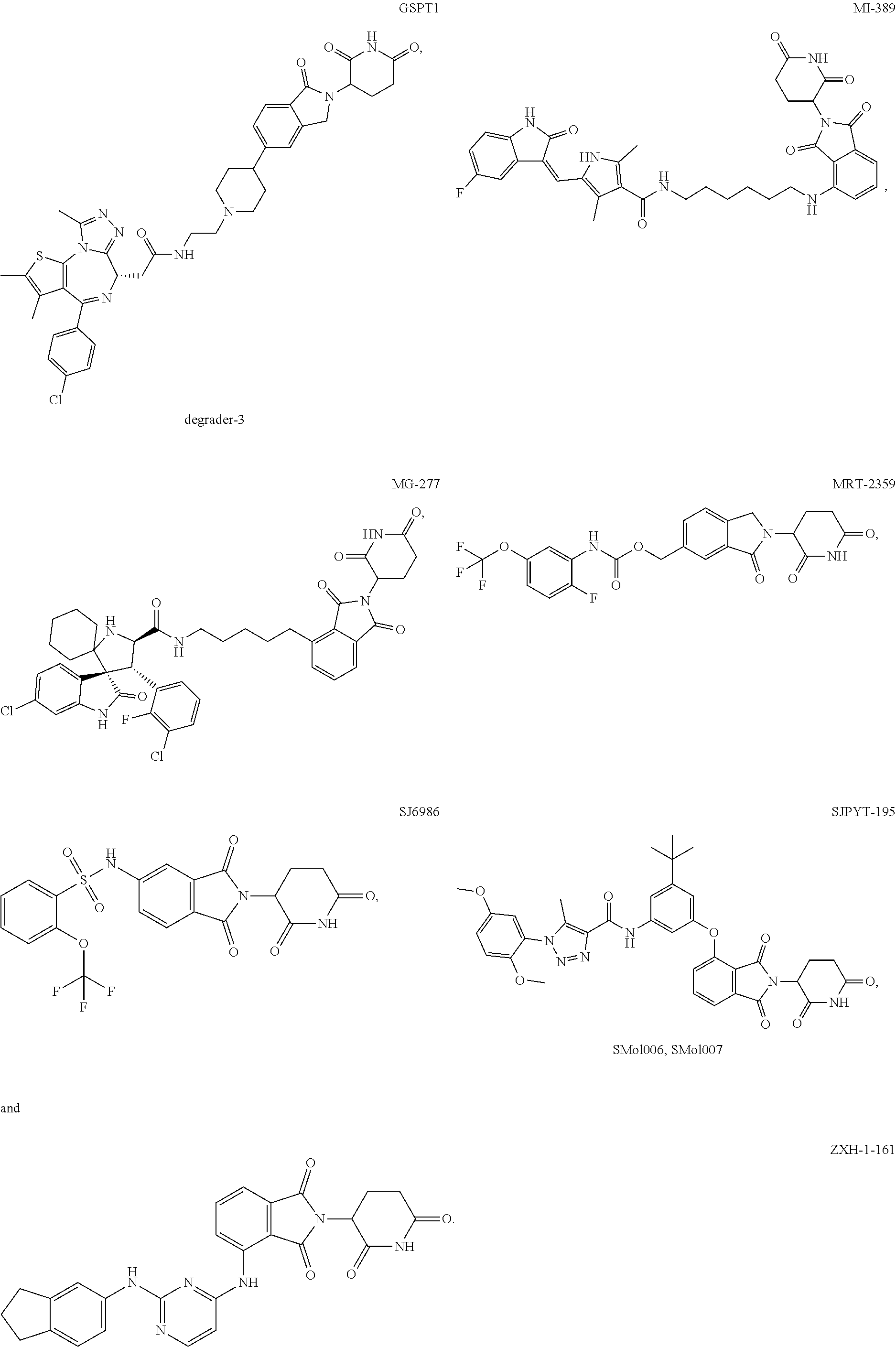

In certain embodiments, administering the composition causes apoptosis in skin-homing T cells.

In certain embodiments, administering the composition causes apoptosis in the skin tissue resident memory T cells (TRMs).

In certain embodiments, the composition is a pharmaceutical composition.

In certain embodiments, the composition is administered topically, orally, or parentally.

In certain embodiments, the subject is a mammal, optionally a human.

In another aspect, provided is a pharmaceutical composition, comprising:
a compound that downregulates an expression level and/or an activity of GSPT1; and
a pharmaceutically acceptable carrier,
wherein the pharmaceutical composition is formulated as a topical formulation.

In another aspect, provided is a method for treating, ameliorating, and/or preventing an autoimmune or inflammatory disease or disorder in a skin in a subject in need thereof, the method comprising administering an effective amount of the pharmaceutical composition comprising:
a compound that downregulates an expression level and/or an activity of GSPT1; and
a pharmaceutically acceptable carrier,
wherein the pharmaceutical composition is formulated as a topical formulation.

In certain embodiments, the autoimmune or inflammatory disease or disorder is selected from the group consisting of alopecia areata, graft-versus-host disease (GVHD), lichen planus, lupus erythematosus, scleroderma, localized scleroderma such as morphea, psoriasis, psoriasis-related conditions such as psoriatic arthritis, Stevens-Johnson syndrome, urticarial vasculitis, vitiligo, atopic dermatitis (eczema), and bullous pemphigoid.

In certain embodiments, the compound comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

In certain embodiments, the composition is formulated for a T cell-specific delivery method.

In certain embodiments, composition comprises a lipid nanoparticle (LNP) conjugated to an antibody targeting a T cell specific surface antigen.

In certain embodiments, the T cell specific surface antigen is CD5.

In certain embodiments, the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

In certain embodiments, the compound comprises a molecular degrader that links GSPT1 to a ubiquitin ligase.

In certain embodiments, the compound comprises a small molecule inhibitor of GSPT1, and wherein the small molecule inhibitor of GSPT1 comprises at least one selected form the group consisting of SP-3204, CDG0501,

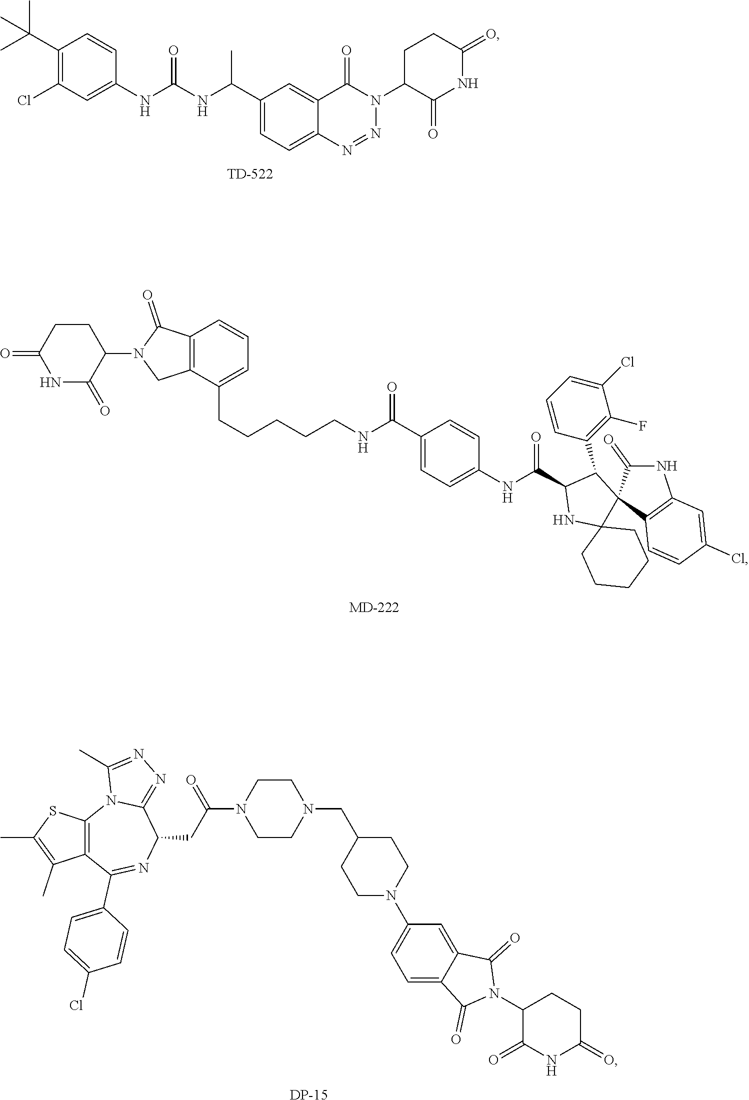 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; "DP-15"]

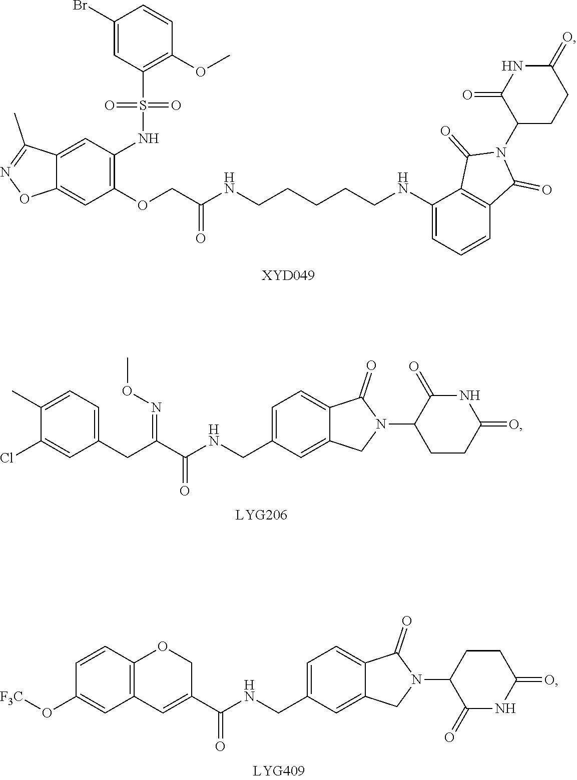

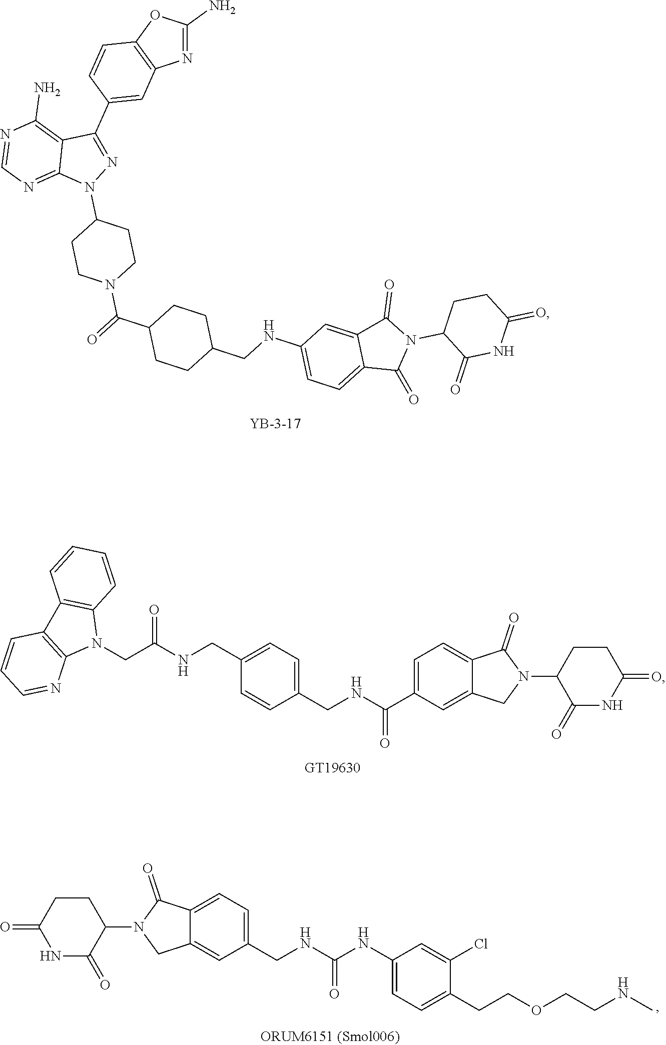 [SMILES: Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; "YB-3-17" ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl]

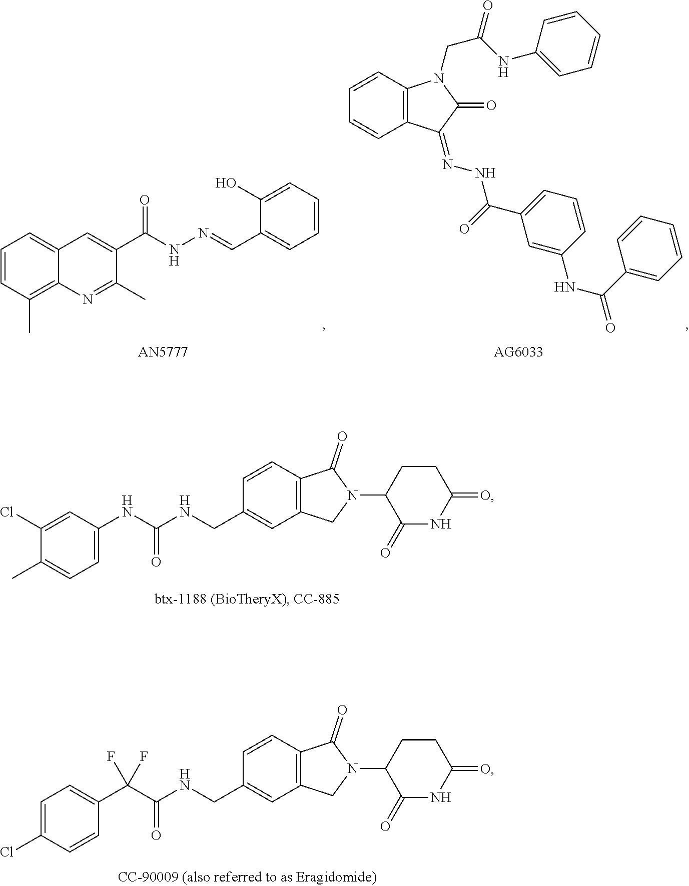 [SMILES: Cc1nc2c(C)cccc2cc1C(=O)N/N=C/c1ccccc1O ; O=C(CN1C(=O)/C(=N\NC(=O)c2cccc(NC(=O)c3ccccc3)c2)c2ccccc21)Nc1ccccc1 ; Cc1ccc(NC(=O)NCc2ccc3c(c2)CN(C2CCC(=O)NC2=O)C3=O)cc1Cl ; O=C1CCC(N2Cc3cc(CNC(=O)C(F)(F)c4ccc(Cl)cc4)ccc3C2=O)C(=O)N1]

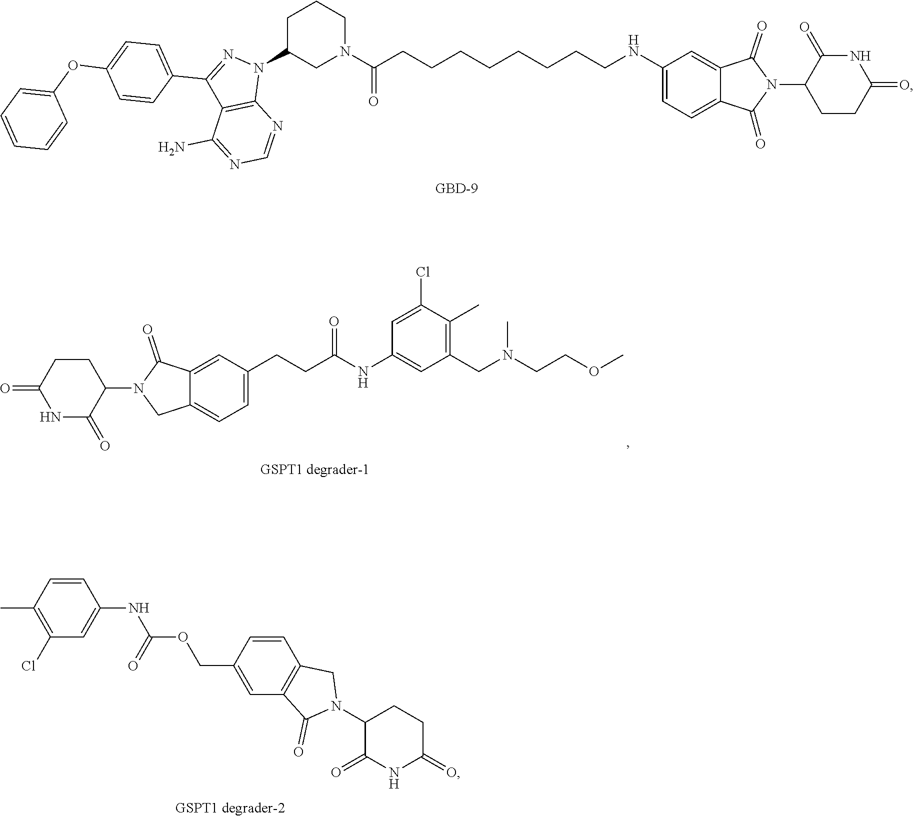

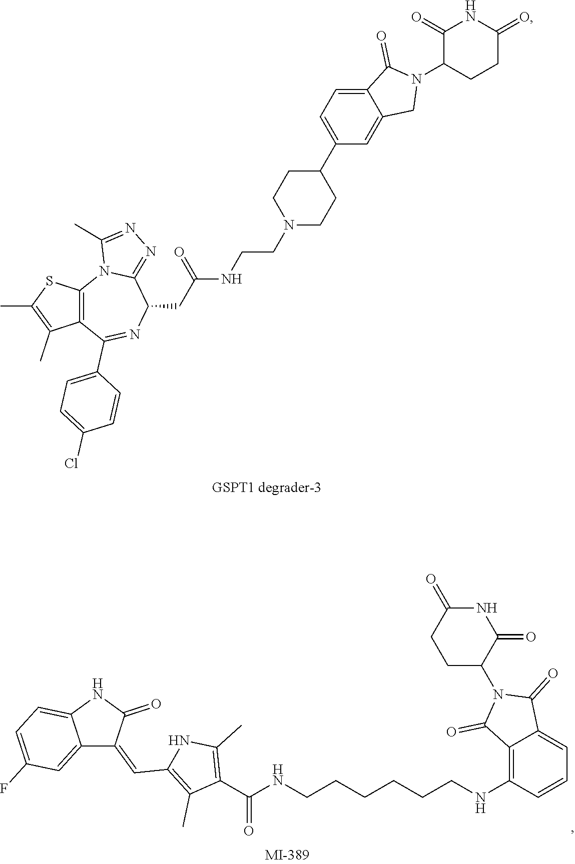 [SMILES: Cc1sc2c(c1C)C(c1ccc(Cl)cc1)=N[C@@H](CC(=O)NCCN1CCC(c3ccc4c(c3)CN(C3CCC(=O)NC3=O)C4=O)CC1)c1nnc(C)n1-2 ; Cc1[nH]c(/C=C2\C(=O)Nc3ccc(F)cc32)c(C)c1C(=O)NCCCCCCNc1cccc2c1C(=O)N(C1CCC(=O)NC1=O)C2=O]

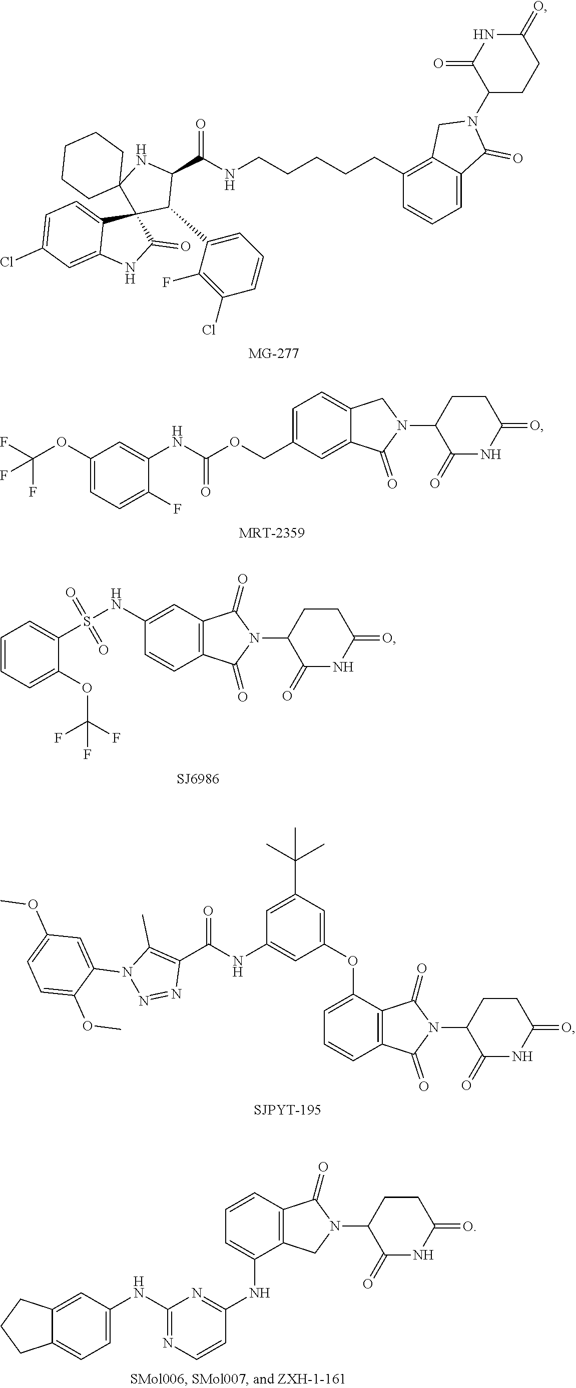 [SMILES: O=C1CCC(N2Cc3c(CCCCCNC(=O)[C@@H]4NC5(CCCCC5)[C@@]5(C(=O)Nc6cc(Cl)ccc65)[C@H]4c4cccc(Cl)c4F)cccc3C2=O)C(=O)N1 ; O=C1CCC(N2Cc3ccc(COC(=O)Nc4cc(OC(F)(F)F)ccc4F)cc3C2=O)C(=O)N1 ; O=C1CCC(N2C(=O)c3ccc(NS(=O)(=O)c4ccccc4OC(F)(F)F)cc3C2=O)C(=O)N1 ; COc1ccc(OC)c(-n2nnc(C(=O)Nc3cc(Oc4cccc5c4C(=O)N(C4CCC(=O)NC4=O)C5=O)cc(C(C)(C)C)c3)c2C)c1 ; O=C1CCC(N2Cc3c(Nc4ccnc(Nc5ccc6c(c5)CCC6)n4)cccc3C2=O)C(=O)N1]

In certain embodiments, the composition is formulated as a cream, a gel, a lotion, an ointment, a patch, a paste, a self-sticking membrane, or a spray.

In another aspect, provided is a method of screening for therapeutic targets for regulating T cell function in a tissue of interest, comprising:
preparing a pool of genetically engineered T cells, wherein all or some of the genetically engineered T cells comprises one or more knocked-out or disrupted genes;
introducing the pool of genetically engineered T cells into a subject carrying the tissue of interest;
collecting T cells from a plurality of tissues including the tissue of interest;
detecting genes that are knocked-out or disrupted in the T cells,
wherein
a gene is determined to inhibit T cell function in the tissue of interest if the gene is knocked-out or disrupted at a higher rate in T cells in the tissue of interest than in other tissues, or
a gene is determined to enhance T cell function in the tissue of interest if the gene is knocked-out or disrupted at a lower rate in T cells in the tissue of interest than in other tissues.

In certain embodiments, the genetically engineered T cells and the tissue of interest is originated from a first species, and wherein the subject is from a second species different from the first species.

In certain embodiments, the first species is human, and the second species is a non-human species, optionally an immunodeficient non-human species.

In certain embodiments, the method screens genes that cause or are associated with a T cell-mediated autoimmune disease in the tissue of interest, and wherein a gene is determined to cause or associate with the T cell-mediated autoimmune disease in the tissue of interest if the gene is knocked-out or disrupted at a higher rate in T cells found in the tissue of interest than in other tissues.

In certain embodiments, the method screens genes that promote T cell mediated anti-cancer immunity, and wherein a gene is determined to promote T cell mediated anti-cancer immunity against a cancer tissue if the gene is knocked-out or disrupted at a lower rate in T cells found in the cancer tissue than in other tissues.

In certain embodiments, preparing the pool of genetically engineered T cells comprises editing T cells with a CRISPR gene editing system comprising a gRNA library comprising gRNAs for targeting a plurality of genes in the T cells.

In certain embodiments, the gRNA library is a genome-wide gRNA library.

In certain embodiments, the method of the above aspects or embodiments or any aspect or embodiment disclosed herein further comprises confirming that the gene identified in the screening inhibits or enhances T cell function.

In certain embodiments, confirming that the gene identified in the screening inhibits or enhances T cell function comprises:
introducing T cells into a subject carrying the tissue of interest;
isolating a plurality of tissues including the tissue of interest;
detecting a level of the gene identified in the screening,
wherein the gene is confirmed to inhibit T cell function in the tissue of interest if the level of the gene is reduced in the tissue of interest as compared to that in other tissues, and
wherein the gene is confirmed to enhance T cell function in the tissue of interest if the level of the gene is increased in the tissue of interest as compared to that in other tissues.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1B illustrate certain aspects of T-cell differentiation/distribution study using a human skin xenograft mouse model, in accordance with some embodiments. FIG. 1A: the immunodeficient NSG mice were grafted with human skin xenografts at day −50. At day 0, between 6 and 7×10⁶ primary human T cells were introduced into the mice intravenously. At day 20, the skin xenografts were harvested. FIG. 1B: human T cells introduced into the mice were detected by staining human CD3. The human T cells were found to be in the human skin graft or remain in compartments such as spleen, blood, or bone marrow.

FIG. 2 illustrates certain aspects of a genome-wide in vivo CRISPR/Cas9 knock-out screen of primary human T cells infiltrating human skin xenografts, in accordance with some embodiments. Specifically, primary human T cells were edited with the CRISPR/Cas9 system and gRNAs targeting genome-wide genes to knock-out one gene per T cell in a random fashion. Using example antigens such as CD45 and TRAC, it was confirmed that the edit efficiency was more than 85%. Furthermore, it was confirmed that more than 375 T cells were transduced for each of the gRNAs. The edited T cells were then introduced into NSG mice in the manner as described in FIGS. 1A-1B.

FIG. 3 demonstrates that T cells in skin and spleen/bone marrow (BM) display distinct expression profiles, in accordance with some embodiments. Human T cells from skin graft and spleen/bone marrow (BM) were isolated by digestion and flow cytometry, followed by single-cell whole transcriptome sequencing. Displayed is a UMAP dimensionality reduction that is color-coded with respect to the tissue from which the T cells were isolated. Green=T cells from skin, red=T cells from bone marrow (BM), blue=T cells from spleen. Each dot represents a cell, red and blue cells are overlapping, indicating that spleen and bone marrow T cells demonstrate similar expression profiles. T cells from the skin cluster separately. Proximity of clusters represents similarity in terms of expression profile.

FIGS. 4A-4B depict the whole genome CRISPR knock-out screen that identifies genes that are essential for skin-homing T cells, in accordance with some embodiments. In FIG. 4A, each dot represents a gene. Y-axis: fold skin-depletion of T cells with disruption of a given gene. X-axis: gene knock-outs in T cells ranked in descending order based on how strong their gene KO is depleting T cells from the skin. GSPT1 demonstrates the 4th strongest depletion of T cells from the skin when disrupted in T cells. FIG. 4B demonstrates that GSPT1 disruption resulted in gRNA depletion from the skin, in accordance with some embodiments. Top panel: histogram of control gRNA distribution; x-axis: zlfc=z-score log 2 fold change. Positive value indicates that T cells expressing these gRNAs (i.e. deficient in these genes) are enriched in the skin; conversely, negative values indicate that T cells expressing these gRNAs (i.e. deficient in these genes) are depleted from the skin. Control non-targeting gRNAs exhibit a normal distribution with a mode around 0, i.e. they are as a group neither enriched, nor depleted from the skin. Bottom panel: gRNAs targeting GSPT1 indicated by orange bars demonstrate a depletion from the skin, i.e. T cells with disrupted GSPT1 gene are depleted from the skin. Gray shadowing represents distribution of control gRNAs.

FIGS. 5A-5B: Skin T cells substantially increase their GSPT1 expression, demonstrating an important role of GSPT1 in T cell tissue adaptation. FIG. 5A: Single cell sequencing of skin and circulating T cells from humanized model mice. Skin T cells demonstrate increased expression of GSPT1 compared to circulating T cells (darker color indicates higher expression). Quantification of expression difference: 2.1 log 2 FC, adjusted p value <0.0001. FIG. 5B: Single cell expression of skin and circulating T cells from human GVHD patients. Skin T cells demonstrate higher expression of GSPT1 compared to circulating T cells.

FIGS. 6A-6C demonstrate that, while GSPT1-depletion resulted in T cell death, circulating T cells were significantly more resistant to GSPT1-depletion, in accordance with some embodiments. FIGS. 6A-6B: treatment of acute myeloid leukemia (AML) cells (FIG. 6A) and primary leukemia cells (FIG. 6B) with a GSPT-1 degrader CC-90009 resulted in dose-dependent depletion of the cells. (Data extracted from J Clin Invest. 2022; 132 (16): e153514). FIG. 6C: in contrast to the AML cells, circulating T cells required about 100-fold higher dose of the GSPT-1 degrader to be depleted. The GSPT-1 degrader used in FIG. 6C is C90009, the same drug as in FIGS. 6A-6B. Blue curve: kill curve with MRT-2359. Primary human T cells were incubated with indicated doses for 3 days followed by viability quantification using a standard celltiter glow assay. Survival was quantified relative to T cells from the same donor treated with DMSO.

FIG. 7 illustrates a conceptual overview of the key role played by GSPT1 as a critical regulator of translation termination, especially in tissue-resident T cells (TRM) especially in the skin.

FIGS. 8A-8D depict a UMAP of T cells isolated from humanized model mice (FIG. 8A), demonstrating distinct transcriptional profiles between skin T cells and circulating T cells (isolated from spleen and bone marrow). FIGS. 8B/8C Skin T cells express bona fide TRM markers at the RNA and protein level. FIG. 8D Positive TRM module score in skin T cells, indicating their bona fide TRM identity.

FIGS. 9A-9B depict an in vivo genome-wide CRISPR-Cas9 knock-out (KO) screen. FIG. 9A Schematic design of in vivo CRISPR/Cas9 knock-out screen. FIG. 9B Validation of knock-out of proof-of concept gene T cell receptor alpha (TRAC). >95% of T cells demonstrate loss of surface T cell receptor. FIG. 9C T cells with disrupted GSPT1 gene demonstrate strongest impairment to become TRM. All labeled genes in C with FDR <0.05.

FIGS. 10A-10D illustrate a study confirming increased GSPT1 expression in TRM in the dataset of the present studies and in an independent human graft-versus-host disease cohort. FIG. 10A Increase in GSPT1 expression in TRM vs. circulating T cells. FIG. 10B T cells with GSPT1-KO are depleted from the skin. FIG. 10C GSPT1 deficiency does not impair proliferation of circulating T cells after activation (n=3). FIG. 10D TRM demonstrate increased cell death after GSPT1 inhibition in vitro. **p<0.01, ***p<0.001, Mann-Whitney test, BH-corrected where appropriate.

FIGS. 11A-11C illustrate a stable isotope labeling by amino acids in culture (SILAC) study to demonstate the impact of GSPT1 on the TRM translatome and overall translation efficiency, we conducted proof-of-concept SILAC experiments using primary human T cells (isolated from peripheral blood) from two donors: FIG. 11A Schematic of SILAC studies. FIG. 11B Protein turnover (fraction newly synthesized) within 24 hours. FIG. 12C GSPT1 KO T cells (circulating) demonstrate a decrease in polysome to monosome ratio, indicating less efficient protein translation. Representative of three biological replicates.

FIGS. 12A-12B depict the in vivo targeting of TRM with lipid nano-particles (LNPs). FIG. 12A CD5-targeting LNP containing GFP mRNA transfect human T cells in a dose-dependent manner. FIG. 12B Sanger chromatograms of GSPT1 gene of wild-type and base-editor treated T cells, resulting in mutated splice acceptor site and >95% reduction in GSPT1 protein. FIGS. 12C/12D illustrate a timeline of a proof-of-concept in vivo experiment, demonstrating GFP expression in >95% of T cells by 2-photon microscopy (white arrows) and flow cytometry.

FIGS. 13A-13B illustrate degradation of GSPT1 in primary human T cells using CC-90009 and MRT-2359. FIG. 13A is a Western blot analysis of GSPT1 protein in primary human T cells isolated from peripheral blood. T cells were treated with indicated concentrations of GSPT1 degrader for 24 hours in vitro. GSPT1 protein was detected with a monoclonal anti-GSPT1 antibody. FIG. 13B is a graph quantifying GSPT1 protein remaining after degrader treatment as shown in FIG. 13A compared to a DMSO negative control. Both CC-90009 and MRT-2359 cause dose-dependent downregulation of GSPT1 protein.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Autoimmune/inflammatory diseases/disorders involve T cell mediated tissue damage. For example, psoriasis is an incurable inflammatory skin disease that is associated with systemic complications, reduced quality of life, increased mortality, and affects over 125 million individuals worldwide. Due to their chronic and incurable nature, these diseases contribute to systemic complications, reduced life expectancy, and severely diminished quality of life. Long-term disease management relies on systemic immunosuppression, which unfortunately raises the likelihood of infections and malignancies. While therapies targeting cytokines and intracellular signaling (JAK, TYK2 inhibitors) have improved symptom control, they often lose efficacy over time, forcing dose escalations or treatment changes. Thus, breaking this cycle of treatment and relapse by targeting the root cause of disease chronicity represents a critical unmet need.

While both innate and adaptive immune populations are implicated in the pathogenesis of skin-related autoimmune diseases such as psoriasis, skin-homing T lymphocytes and skin tissue-resident memory T cells (TRM) in particular are now considered the main drivers of these diseases: when non-lesional skin from patients with psoriasis (but not normal control skin) is grafted onto immunocompromised mice, the skin develops a psoriatic phenotype indicating that a pre-existent, non-circulatory pathogenic T cell population exists in the skin graft and is sufficient to cause psoriatic immunopathology. Furthermore, human clinical trials evaluating the efficacy of blocking T cell egress from the vasculature into the skin by targeting E-selectin with a monoclonal antibody failed to improve psoriasis. These results indicate that ongoing influx of T lymphocytes into psoriatic plaques is not necessary for persistent disease and that resident T cells are sufficient to maintain disease activity.

Similarly, atopic dermatitis (AD) is a common chronic inflammatory skin condition, impacting 15% of children and 7% of adults across the United States. Notably, a significant fraction of those affected-approximately 20-30%-suffer from moderate to severe forms of the disease, underscoring the pressing need for more effective management strategies that provide lasting remissions. Atopic dermatitis manifests as a systemic inflammatory disorder and has been implicated in elevating the risk of cardiovascular complications, which is further highlighted by the observation that severe atopic dermatitis is associated with increased mortality. T cells, as well as T cell-derived cytokines, have long been considered as a pathogenic factor.

The persistent, relapsing nature of these skin diseases is driven largely by tissue-resident memory T cells (TRM). Even after apparent resolution, TRM remain in quiescent lesions and rapidly reignite inflammation upon minimal stimulation, explaining why even potent systemic therapies rarely achieve durable remission. Their resistance stems from specialized adaptations allowing them to survive long-term in the nutrient- and oxygen-limited skin microenvironment. TRM also rarely re-enter circulation, thus evading many conventional systemic treatments.

The conventional understanding of T cell mediated tissue damage in these conditions centers around T cell receptor (TCR) specificity and cytokine signaling as the primary determinant for niche entry and maintenance. However, skin-homing T cell clones persist for years in their niche and do not demonstrate signs of T cell exhaustion (a state characterized by progressive loss of T cell function due to chronic antigen stimulation), indicating that additional adaptation must occur to facilitate persistence. Although advancements in cytokine-targeting therapeutics have revolutionized chronic skin disease control, these approaches do not provide a cure, mainly due to their inability to target the persistence mechanisms of skin-homing T cells in the skin niche.

In an unexpected turn of results from a genome-wide CRISPR-Cas9 knockout screen, the studies described herein (“the present studies”) identified the translation termination factor GSPT1 (or G1-to-S Phase Transition 1) as indispensable for TRM survival due to their high protein synthesis levels which is required in nutrient-poor skin tissues. Sustaining the TRM state in situ demands continuous production of tissue-retention molecules, core residency transcription factors, and metabolic enzymes tailored to hypoxia and nutrient scarcity—a biosynthetic burden unmatched by circulating T cells. Indeed, while GSPT1 expression is required by TRM cells, its absence is well-tolerated by circulating T cells.

GSPT1 is the sole isoform in T lymphocytes. It forms a complex with eRF1 to recognize stop codons and release nascent peptides from ribosomes, ensuring accurate translation termination and preventing aberrant protein accumulation. Notably, effective termination is integral to overall “translation efficiency”, the ability of each transcript to produce maximal functional protein with minimal wasted energy. Data presented herein show that depleting GSPT1 in healthy circulating T cells does not affect proliferation or survival, aligning with clinical observations that circulating T cells tolerate partial GSPT1 loss. These data strongly suggest that TRM in human skin, which are forced to function under hypoxic, nutrient-poor conditions, are uniquely vulnerable to GSPT1 inhibition. This vulnerability reflects TRM's dependence on GSPT1 to maintain their identity-defining proteome under energetic constraints in the skin, making it a promising tissue-specific therapeutic target that could eliminate TRM while sparing circulating T cells.

GSPT1's significant upregulation in skin T cells, as confirmed through independent datasets, highlights its role in the adaptation and persistence of skin-homing T cells in the skin environment. Patients with psoriasis and graft-versus-host disease (GVHD) exhibit high GSPT1 levels in skin T cells, in stark contrast to circulating T cells.

Indeed, GSPT1 depletion dose-dependently induces T cell apoptosis. Using two distinct GSPT1 inhibitors, the present study demonstrated that the inhibition of GSPT1 can achieve dose-dependent killing, underscoring the therapeutic value of GSPT1 blockade in diseases characterized by the dysregulation of skin-resident T cells. Additionally, the present study establishes that disruption of the GSPT1 gene results in the inability of T cells to persist in the skin niche. Given that GSPT1 is the hallmark of a specific adaptation program in skin-homing T cells, targeting GSPT1 offers an innovative therapeutic approach that treats, ameliorates and/or prevents autoimmune/inflammatory skin disease/disorder, which selectively eliminates the persisting T cells from the skin niche rather than managing the damaging effects by these T cells. As such, the method developed in the present study can achieve long-lasting remissions in autoimmune/inflammatory skin disease/disorder caused by or involving skin-homing T cells.

Accordingly, in certain aspects, the present invention is directed to a method of reducing skin-homing T cells in a skin tissue. The invention also includes a method of treating, ameliorating, and/or preventing autoimmune skin diseases, including but not limited to alopecia areata, graft-versus-host disease (GVHD), lichen planus, lupus erythematosus, scleroderma (including localized forms such as morphea), psoriasis and related conditions such as psoriatic arthritis, Stevens-Johnson syndrome, urticarial vasculitis, vitiligo, atopic dermatitis (eczema), and bullous pemphigoid.

Furthermore, it is expected that the depletion of T cells from tissues being damaged by T cells reside in the tissues have applications in other autoimmune conditions where T cells play a pivotal role. Such autoimmune conditions includes, but not limited to: Autoimmune thyroid diseases such as Graves' disease and Hashimoto's thyroiditis; rheumatologic conditions such as rheumatoid arthritis, systemic lupus erythematosus, and Sjögren's syndrome, granulomatosis with polyangiitis; gastrointestinal diseases such as celiac disease, inflammatory bowel diseases (including Crohn's disease and ulcerative colitis), and autoimmune hepatitis; neurological conditions such as multiple sclerosis, Guillain-Barré syndrome, and myasthenia gravis; type 1 diabetes mellitus, where autoreactive T cells target pancreatic β-cells; autoimmune myocarditis, which involves T cell-mediated damage to cardiac tissue; autoimmune uveitis, a T cell-driven inflammatory condition affecting the eyes; systemic sclerosis, characterized by T cell dysregulation contributing to fibrosis of the skin and internal organs; and, autoimmune cytopenias, including immune thrombocytopenia and autoimmune hemolytic anemia.

This broader perspective emphasizes the versatility of the method in addressing T cell-mediated pathologies beyond the skin, offering therapeutic benefits across a spectrum of autoimmune disorders.

Mis-regulation of T cell homing can lead to various medical challenges. In autoimmune disorders, an excess of T cells erroneously localizes and activates within tissues, causing damage. Conversely, certain cancers, such as those of the breast, ovary, prostate, pancreas, and brain, often display unusually low T cell infiltration, contributing to their classification as ‘cold tumors’. Our screening method is designed to pinpoint genes that govern T cell activity within these tissues, highlighting potential therapeutic targets.

Testing T cell infiltration in native human tissues, particularly skin, using the screening method described herein, has distinct advantages over tumor models using cell lines, or patient-derived tumor xenografts. The methods disclosed herein provide a more accurate representation of T cell behavior and interaction within their physiological context. Furthermore, knockout (KO) studies in this setting can reveal T cells with heightened in vivo fitness, implying that genes disrupted in these KOs can serve as valuable targets for cancer immunotherapy, enhancing T cell presence and activity in tumor environments.

Accordingly, in some embodiments, the present invention is further directed to a method of screening therapeutic targets for regulating T cell function in a tissue of interest.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments±5%, in certain embodiments±1%, in certain embodiments±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

### Methods of Screening Therapeutic Targets for Regulating T Cell Function

In some aspects, the present invention is directed to a method of screening therapeutic targets for regulating T cell function, such as in a tissue of interest.

In some embodiments, the method comprises:
preparing a pool of genetically engineered T cells, in which all or some of the T cells have one or more genes being knocked-out or disrupted;
introducing the pool of genetically engineered T cells into a subject carrying the tissue of interest;
harvesting a plurality of tissues including the tissue of interest;
detecting genes that are knocked-out or disrupted in the plurality of tissues.

In some embodiments, a gene is determined to inhibit T cell function in the tissue of interest if the gene is knocked-out or disrupted at a higher rate in T cells found in the tissue of interest than in other tissues.

In some embodiments, a gene is determined to enhance T cell function in the tissue of interest if the gene is knocked-out or disrupted at a lower rate in T cells found in the tissue of interest than in other tissues.

In some embodiments, the pool of genetically engineered T cells is prepared by subjecting T cells, such as primary T cells, to a gene-editing method that knock-out or otherwise disrupt one or more random genes in each of the T cells.

In some embodiments, on average, in the pool of genetically engineered T cells, each T cell has about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2 gene(s) knocked-out or otherwise disrupted.

In some embodiments, the pool of genetically engineered T cells is prepared by editing a pool of T cells with a CRISPR system that includes a guide RNA (gRNA) library including gRNAs for targeting all or some of the genes across the entire genome. In some embodiments, the gRNA library has a coverage about 1 gene or more, about 10 genes or more, about 100 genes or more, about 500 genes or more, about 1,000 genes or more, about 2,000 genes or more, about 2,000 genes or more, about 3,000 genes or more, about 4,000 genes or more, about 5,000 genes or more, about 6,000 genes or more, about 7,000 genes or more, about 8,000 genes or more, about 9,000 genes or more, about 10,000 genes or more, about 12,000 genes or more, about 14,000 genes or more, about 16,000 genes or more, about 18,000 genes or more, about 20,000 genes or more, about 25,000 genes or more, about 30,000 genes or more, or all known protein-encoding genes in the species that the T cells originate from.

In some embodiments, in the pool of genetically engineered T cells, about 1% or more, about 2% or more, about 3% or more, about 5% or more, about 10% or more, about 20% or more, about 30% or more, about 40% or more, about 50% or more, about 60% or more, about 70% or more, about 80% or more, about 90% or more, or about 95% or more of the T cells have one or more genes knocked-out or otherwise disrupted.

In some embodiments, the genetically engineered T cells and the tissue of interest is originated from a first species, and the subject is from a second species, such as a second species which is different from the first species. In some embodiments, the tissue of interest is originated from a human subject. In some embodiments, the subject into which the genetically engineered T cells are introduced is a non-human subject, such as a rodent subject (e.g., mouse, rat, rabbit, hamster, etc.), and/or an immunodeficient subject (such as a genetically engineered immunodeficient subject). Non-limiting examples of tissues of interest that can be xenografted from the subject from the first species to the subject of the second species include a skin tissue, a cancer tissue, and the like.

In some embodiments, the method screens genes that cause or are associated with a T cell-mediated autoimmune disease in the tissue of interest. In some embodiments, a gene is determined to cause or associate with the T cell-mediated autoimmune disease in the tissue of interest if the gene is knocked-out or disrupted at a higher rate in T cells found in the tissue of interest than in the other harvested tissues.

In some embodiments, the method screens genes that promote T cell mediated anti-cancer immunity. In some embodiments, a gene is determined to promote T cell mediated anti-cancer immunity against a cancer tissue if the gene is knocked-out or disrupted at a lower rate in T cells found in the cancer tissue than in other tissues.

In some embodiments, the method further comprises confirming that the gene identified in the screening inhibits or enhances T cell function in the tissue of interest.

In some embodiments, confirming that the gene identified in the screening inhibits or enhances T cell function in the tissue of interest comprises:
introducing T cells into a subject carrying the tissue of interest;
isolating a plurality of tissues including the tissue of interest; and
detecting a level of the gene identified in the screening,

In some embodiments, the gene is confirmed to inhibit T cell function in the tissue of interest if the level of the gene is reduced in the tissue of interest as compared to that in other tissues.

In some embodiments, the gene is confirmed to enhance T cell function in the tissue of interest if the level of the gene is increased in the tissue of interest as compared to that in other tissues.

In some embodiments, the tissue of interest is a skin tissue. In some embodiments, the tissue of interest is a xenograft skin tissue.

In some embodiments, the tissue of interest is a cancer tissue. In some embodiments, the tissue of interest is a xenograft cancer tissue.

Apart from skin and tumor tissues, several other types of human tissues can be xenografted into non-human subjects (such as mice, such as immunodeficient non-human subjects, such as immunodeficient mice) for various research purposes. These include, but are not limited to: Bone marrow—to study hematopoiesis and immune cell generation; liver—for research on metabolism, drug detoxification, and liver disease; kidney—to investigate renal function and disease; pancreas—often used in diabetes research to study insulin production; h eart—for cardiovascular research, including heart disease mechanisms; brain—in neurological research, particularly for studying neurodegeneration; breast—to study normal breast tissue development or breast cancer; prostate—for prostate cancer research and normal prostate physiology; ovarian—in studies of ovarian function, fertility, and ovarian cancer; and, intestinal—to explore gut biology, microbiome interactions, and intestinal disorders.

These xenografts can help model human disease in a controlled environment, allowing researchers to observe disease progression and test therapeutic interventions.

Methods of Reducing T Cell Presence in Skin and Methods of Treating, Ameliorating, and/or Preventing Autoimmune and Inflammatory Diseases/Disorders in Skin

In some aspects, the present invention is directed to a method of reducing T cell presence in a skin tissue (also referred to as “skin-homing T cells” herein). In some embodiments, the method reduces the presence of tissue-resident memory T cells (TRMs) in the skin tissue.

In some embodiments, the method includes introducing into the T cells a compound for down regulating GSPT1, such as down regulating a level of GSPT1 and/or an activity of GSPT1.

In some embodiments, the T cell is a T cell in the skin tissue, such as in the skin of a subject. In some embodiments, the T cell is not in the skin tissue, but has the potential to move into the skin tissue, such as from bone marrow or spleen, such as via T cell homing and/or differentiation. In some embodiments, the subject is a mammal, such as a human.

In some aspects, the present invention is directed to a method of treating, ameliorating, and/or preventing autoimmune and inflammatory diseases/disorders in the skin in a subject in need thereof.

In some embodiments, the autoimmune/inflammatory disease/disorder is caused by T cell mediated tissue damage in the skin, or involves T cell mediated tissue damage in the skin as a contributing factor. In some embodiments, the T cell is a tissue-resident memory T cell reside in the skin.

In some embodiments, the autoimmune and inflammatory diseases/disorders is alopecia areata, graft-versus-host disease (GVHD), lichen planus, lupus erythematosus, scleroderma (including localized forms such as morphea), psoriasis and related conditions such as psoriatic arthritis, Stevens-Johnson syndrome, urticarial vasculitis, vitiligo, atopic dermatitis (eczema), or bullous pemphigoid.

In some embodiments, the method comprises administering to the subject an effective amount of a compound for down regulating GSPT1, such as down regulating a level of GSPT1 and/or an activity of GSPT1.

In some embodiments, the subject is a mammal, such as a human.

In some embodiments, the compound that downregulates the expression level or the activity of GSPT1 acts at the genomic level. For example, the expression level of GSPT1 can be down-regulated by gene knockout, such as CRISPR knockout and other knockout techniques.

In some embodiments, the compound that downregulates the expression level or the activity of GSPT1 acts at the transcriptional level or the translational level. For example, the expression level of GSPT1 can be down-regulated by gene knockdown, such as by RNA interference technique, ribozyme knockdown, or CRISPR knockdown.

In some embodiments, the compound that downregulates the expression level or the activity of GSPT1 acts at the post-translational level. For example, the expression level of GSPT1 can be down-regulated by targeted protein degradation, such as proteolysis-targeting chimera (PROTAC), molecular glues and other protein degradation strategies. For example, the activity of GSPT1 can be down-regulated by small molecules inhibitors of GSPT1, antibodies that neutralizes GSPT1, and trans-dominant negative mutant of GSPT1.

In some embodiments, the compound that downregulates the expression level or the activity of GSPT1 includes a small molecule inhibitor of GSPT1, a protein inhibitor of GSPT1, or a compound that downregulates the expression level and/or activity of GSPT1 by RNA interference, by ribozyme, by CRISPR knockout/knockdown, or by producing a trans-dominant negative mutant, and so forth.

In some embodiments, the compound contemplated herein can be delivered by a vector, such as a plasmid or a viral vector. One of ordinary skill in the art would understand that such vectors can be used to deliver compounds in the form of nucleic acids, such as RNA or DNA. Such vectors are described herein below.

In certain embodiments, the compound contemplated herein (including but not limited to nucleic acids) can be more efficiently delivered to the cell nucleus by coupling the compound with the monoclonal anti-DNA antibody 3E10, which penetrates living cells and localizes in the nucleus without causing any apparent harm to the cell (Hansen J E, et al., Intranuclear protein transduction through a nucleoside salvage pathway. J Biol Chem 2007; 282:20790-3; see also WO 2020/047353 and WO 2021/042060, all of which are incorporated herein in their entireties by reference). 3E10 and its single-chain variable fragment (3E10 scFv) have been developed as an intracellular delivery system for macromolecules. After localizing in the cell nucleus, 3E10 scFv is largely degraded within 4 hours, thus further minimizing any potential toxicity.

In certain embodiments, the compounds contemplated herein (including but not limited to nucleic acids) can be more efficiently delivered to the central nervous system using certain lipid nanoparticle formulations known in the art, such as but not limited to those described in Cullis, P. R. et al., Molecular Therapy Vol. 25 No 7 Jul. 2017. See also US20150165039 and WO 2014/008334, all of which are incorporated herein in their entireties by reference.

In certain embodiments, the compounds contemplated herein can be more efficiently delivered to tissue by coupling with certain protein fragments, called “pHLIP” (pH (Low) Insertion Peptide), which allow for the cargo to accumulate in acidic environments within the body. In certain embodiments, a polypeptide with a predominantly hydrophobic sequence long enough to span a membrane lipid bilayer as a transmembrane helix (TM) and comprising one or more dissociable groups inserts across a membrane spontaneously in a pH-dependent fashion placing one terminus inside cell. The polypeptide conjugated with various functional moieties delivers and accumulates them at cell membrane with low extracellular pH. The functional moiety conjugated with polypeptide terminus placed inside cell are translocated through the cell membrane in cytosol. The peptide and its variants or non-peptide analogs can be used to deliver therapeutic, prophylactic, diagnostic, imaging, gene regulation, cell regulation, or immunologic agents to or inside of cells in vitro or in vivo in tissue at low extracellular pH. See also US20080233107, WO2012/021790, US20120039990, US20120142042, US20150051153, US20150086617, and US20150191508, all of which are incorporated herein in their entireties by reference.

### Downregulating GSPT1 Expression and Function by Small Molecule Inhibitors

In some embodiments, the compound that downregulates the expression level or the activity of GSPT1 includes a small molecule that inhibits the activity of GSPT1. As used herein, the term “small molecule” refers to a molecule having a size of less than 2000, 1800, 1600, 1400, 1200, 1000, 800, or 600 daltons.

Various inhibitors of GSPT1 have been identified. For example, Matyskiela et al. (Nature. 2016 Jul. 14; 535 (7611): 252-7), Nishiguchi et al. (J. Med. Chem. 2021, 64, 11, 7296-7311, Publication Date: May 27, 2021), Surka et al. (Blood. 2021 Feb. 4; 137 (5): 661-677), Chang et al. (Med Res Rev. 2024 February), WO 2022/219412 A1, Palacino et al. (Cancer Res (2022) 82 (12_Supplement): 3933), and others describe various small inhibitors of GSPT1.

In some embodiments, small molecule inhibitor comprises a degrader of GSPT1 which promotes the degradation of GSPT1 in T cells.

In some embodiments, the small molecule inhibitor comprises a PROTAC or a Proteolysis Targeting Chimeric Molecule. PROTACs are heterobifunctional nanomolecules that can target protein for ubiquitination and degradation. In certain embodiments, the PROTAC contemplated in the present invention comprises a group that is recognized by the E3 ubiquitin ligase and a group that is recognized by GSPT1. The PROTAC is able to simultaneously bind to the GSPT1 and the E3 ligase. Formation of such trimeric complex formation leads to the transfer of ubiquitins to the GSPT1, marking it for degradation. PROTAC molecules possess good tissue distribution and the ability to target intracellular proteins, thus can be directly applied to cells or injected into animals without the use of vectors. PROTACS useful within the invention can be prepared using any known compound that binds to and/or recognizes and/or inhibits GSPT1, which is linked through a linker to an E3 ubiquitin ligase, such as but not limited to those described in WO 2013/106643, WO 2013/106646, and WO 2019/148055.

In some embodiments, the small molecule inhibitor comprises a molecular glue, which are similar to PROTACs in that they also target proteins for degradation. Molecular glues are small molecules that act as adhesives by making two proteins bind each other, in this case one protein being GSPT1, and the other being a ubiquitin ligase such as the E3 ubiquitin ligase.

Non-limiting examples of small molecule inhibitors of GSPT1 include SP-3204 (U.S. 11,535,603), CDG0501 (Dong et al. (2024) Blood, Vol144, Supplement 1, Page 156),

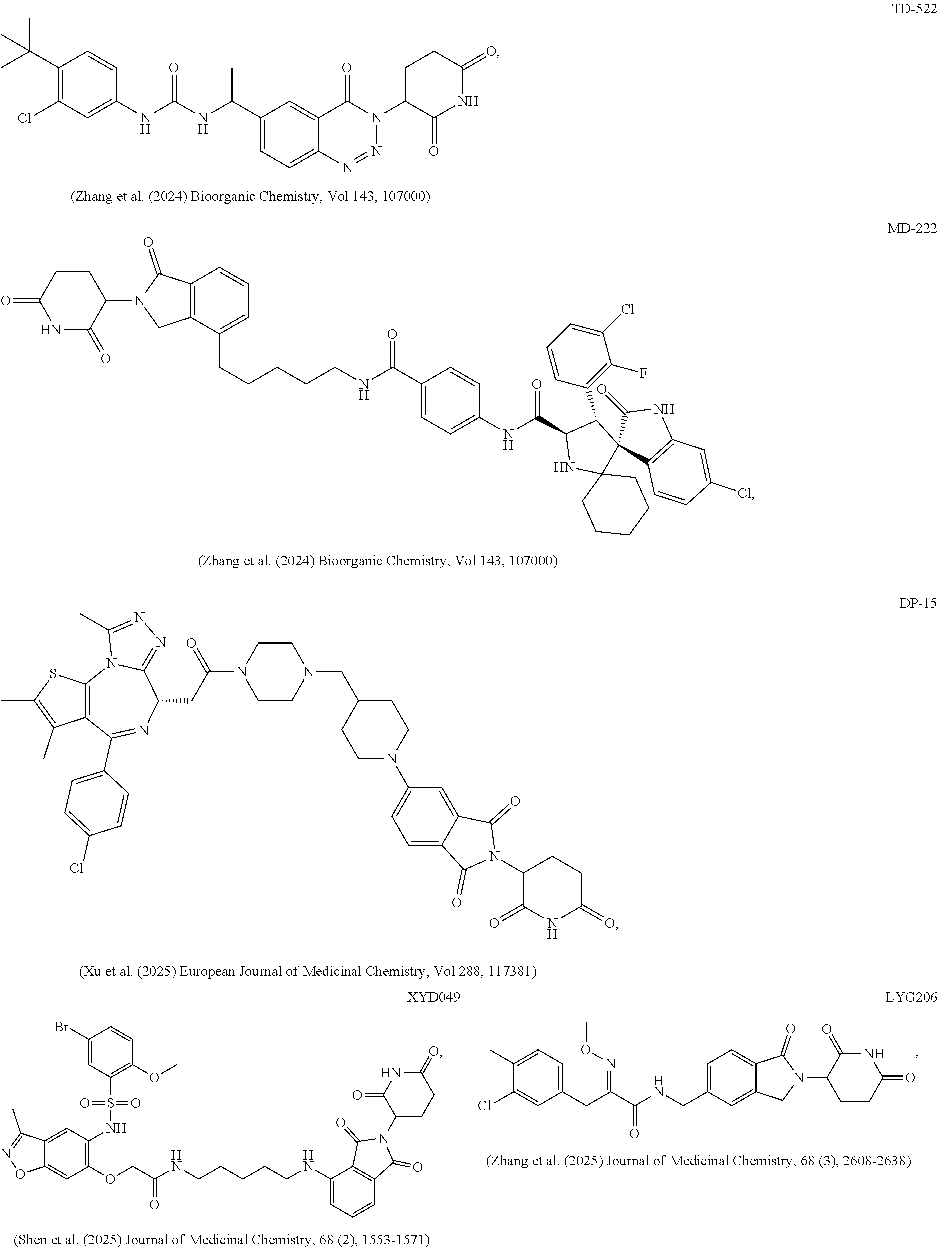 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O ; CO/N=C(\Cc1ccc(C)c(Cl)c1)C(=O)NCc1ccc2c(c1)CN(C1CCC(=O)NC1=O)C2=O]

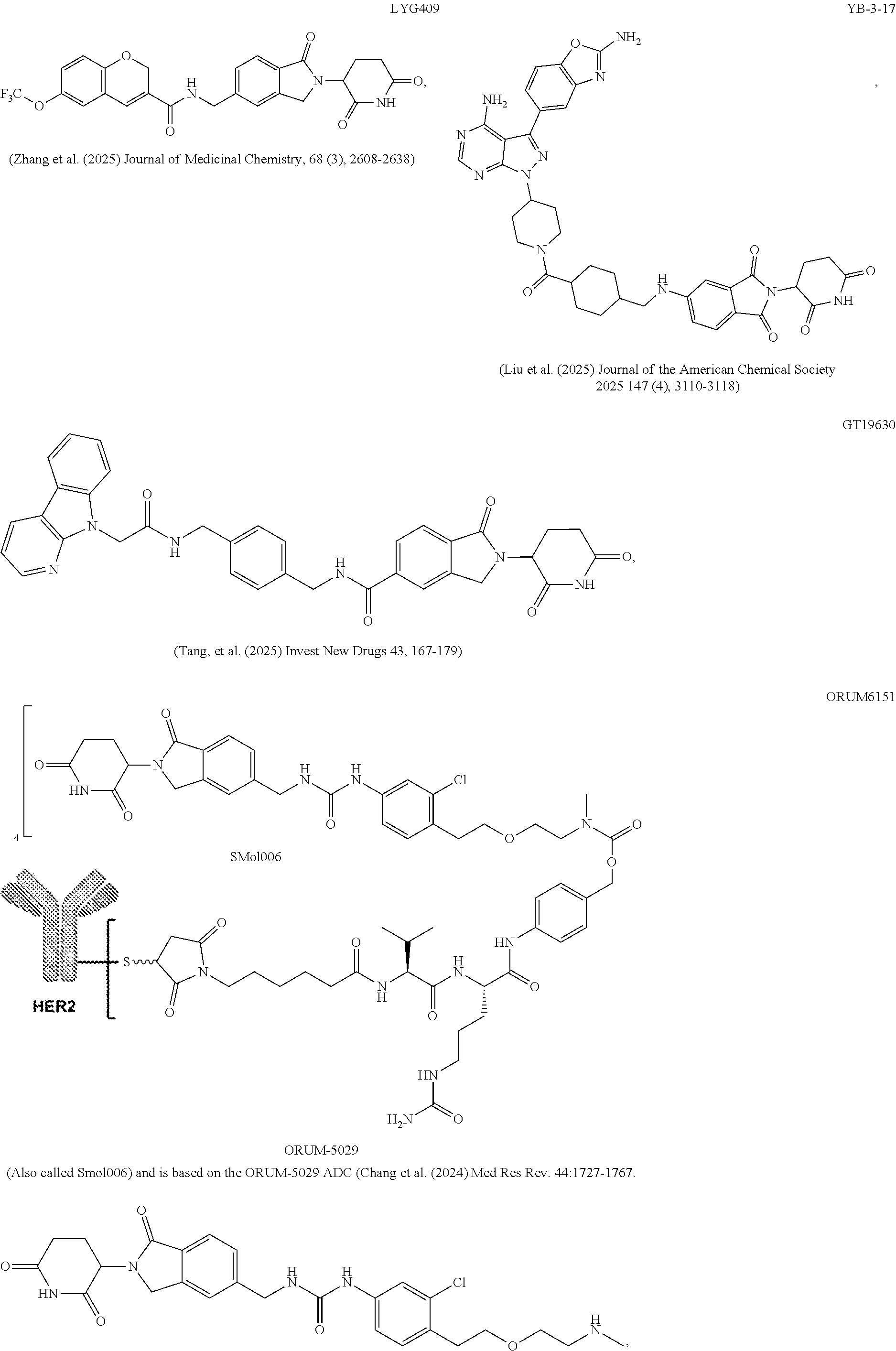

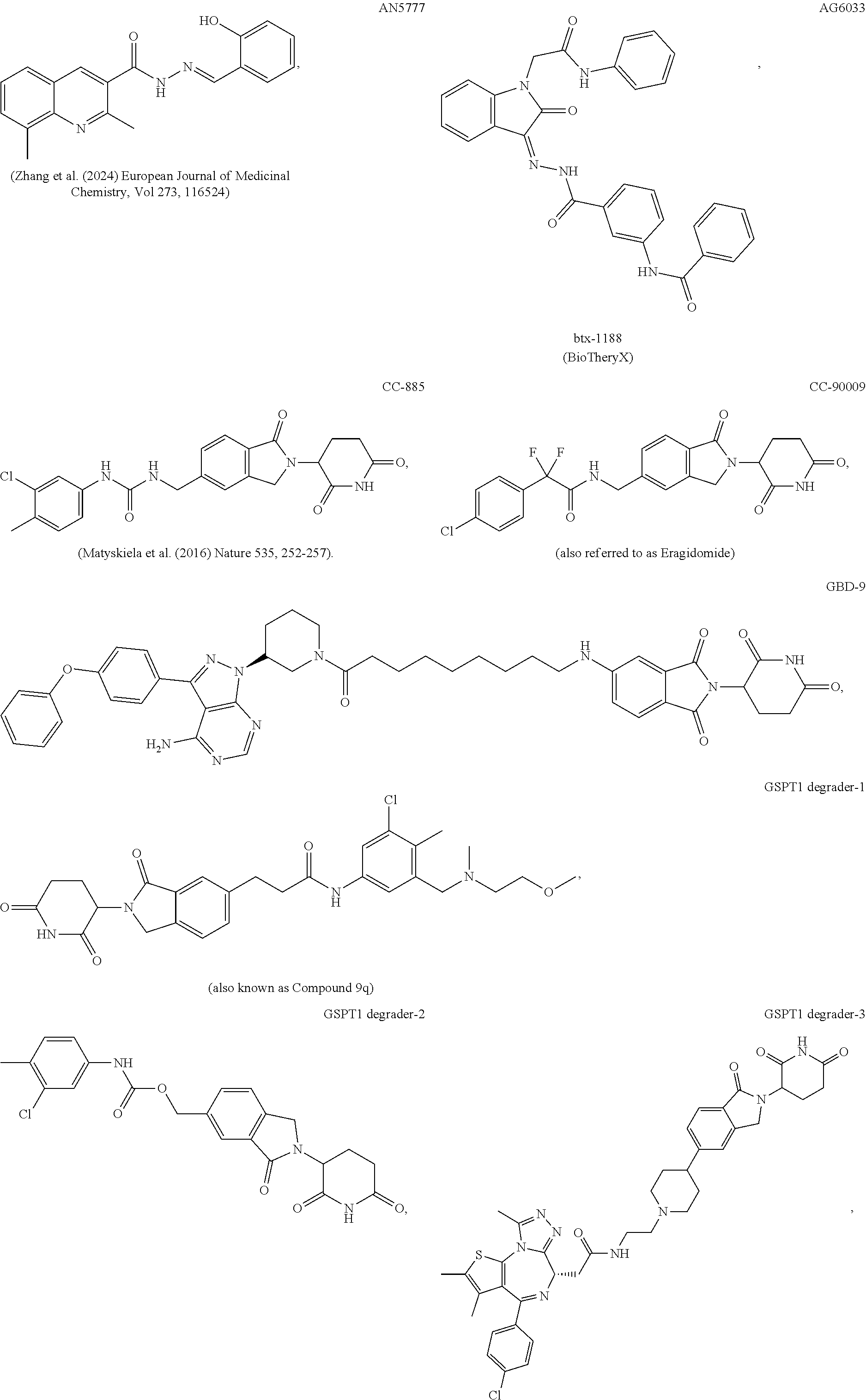

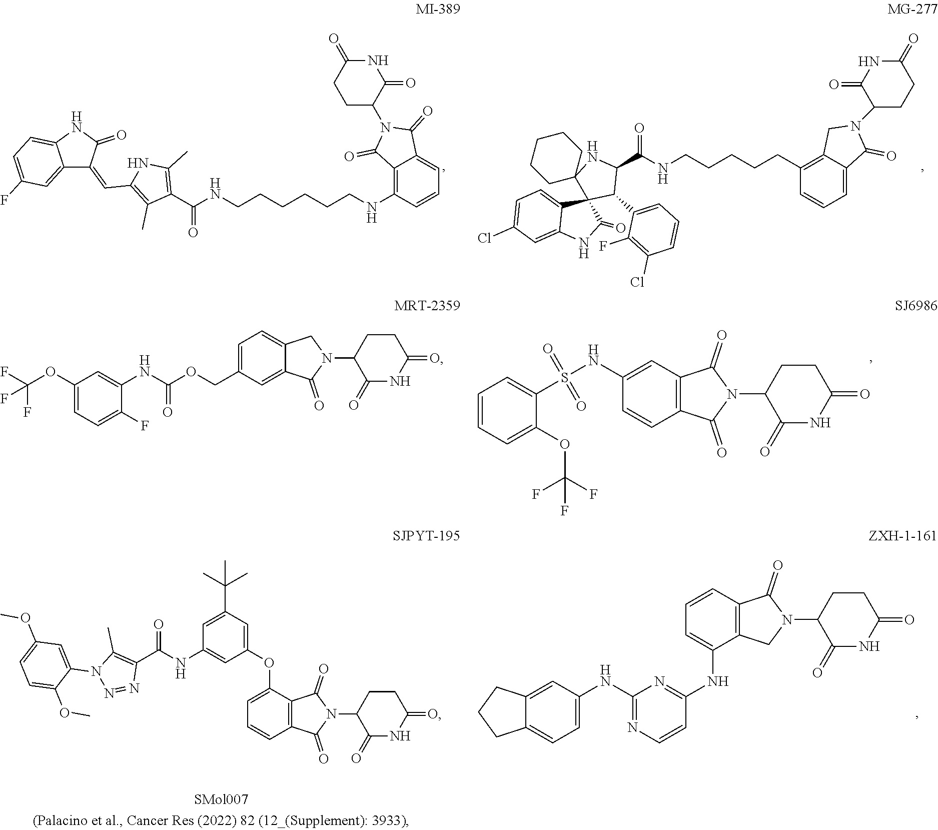 [SMILES: Cc1[nH]c(/C=C2\C(=O)Nc3ccc(F)cc32)c(C)c1C(=O)NCCCCCCNc1cccc2c1C(=O)N(C1CCC(=O)NC1=O)C2=O ; O=C1CCC(N2Cc3c(CCCCCNC(=O)[C@@H]4NC5(CCCCC5)[C@@]5(C(=O)Nc6cc(Cl)ccc65)[C@H]4c4cccc(Cl)c4F)cccc3C2=O)C(=O)N1 ; O=C1CCC(N2Cc3ccc(COC(=O)Nc4cc(OC(F)(F)F)ccc4F)cc3C2=O)C(=O)N1 ; O=C1CCC(N2C(=O)c3ccc(NS(=O)(=O)c4ccccc4OC(F)(F)F)cc3C2=O)C(=O)N1 ; COc1ccc(OC)c(-n2nnc(C(=O)Nc3cc(Oc4cccc5c4C(=O)N(C4CCC(=O)NC4=O)C5=O)cc(C(C)(C)C)c3)c2C)c1 ; O=C1CCC(N2Cc3c(Nc4ccnc(Nc5ccc6c(c5)CCC6)n4)cccc3C2=O)C(=O)N1]

and the like. It is also contemplated that any small molecule degrader of GSPT1 known in the art could be used in the present invention, including, but not limited to, those disclosed in WO2024215130, WO2024201248, WO2024165577, WO2024167423, WO2025016457, WO2024251301, WO2025026218, WO2025051180, CN119431327, CN119019370, WO2024222614, CN118852145, CN118812512, and CN118496213.

In some embodiments, the small molecule inhibitors described herein are conjugated to an antibody molecule, such as an antibody molecule that target T cells, to form antibody-drug-conjugates, which is described elsewhere herein.

### Downregulating GSPT1 by Protein Inhibitors of GSPT1

In some embodiments, the compound that downregulates the expression level or the activity of GSPT1 includes a protein that downregulates the expression level or the activity of GSPT1.

In some embodiments, the protein that downregulates GSPT1 is an antibody, such as a monoclonal antibody or a polyclonal antibody.

Non-limiting examples of monoclonal and/or polyclonal antibodies that target GSPT1 include PA5-62621, MA5-47005 (clone HL1345), MA5-47006 (clone HL1346), and MA5-46543 (clone CL13332) available from Invitrogen (Waltham, MA, USA), AMAB91851 (clone CL13336) available from MilliporeSigma (Burlington, MA, USA), BS-20307R available from Bioss (Woburn, MA, USA), and any humanized derivatives thereof.

In some embodiments, the protein that downregulates the expression level and/or activity of GSPT1 is administered in form of a protein. In some embodiments, the protein that downregulates the expression level and/or activity of GSPT1 is administered in form of a nucleic acid that expresses the protein, such as an expression vector. The expression vector is described in the “Vector” section elsewhere in the instant specification.

Since GSPT1 is an intracellular protein and is therefore inaccessible by antibodies present in an extracellular environment, in some embodiments, the antibody herein is delivered by an intracellular delivery mechanism. Intracellular delivery of therapeutic antibodies is described in, for example, Gaston et al. (Scientific Reports volume 9, Article number: 18688 (2019)) and Li et al. (Advanced Therapeutics, Volume 3, Issue 12, December 2020).

It is worth noting that antibodies that specifically target certain cell types, such as T cells, rather that GSPT1 per se are also useful for the methods herein. As described elsewhere herein, various compounds described herein can be conjugated or otherwise linked to T cell-specific antibodies to increase the target specificity of the compounds.

### Downregulating GSPT1 Protein Expression by RNA Interference

In some embodiments, the compound that downregulates the activity or expression level of GSPT1 includes a nucleic acid that downregulates the activity and/or expression level of GSPT1 by the means of RNA interreference.

In some embodiments, the nucleic acid that downregulates the expression level of GSPT1 by the means of RNA interreference includes an isolated nucleic acid. In other embodiments, the modulator is an RNAi molecule (such as but not limited to siRNA and/or shRNA and/or miRNAs) or antisense molecule, which inhibits GSPT1 expression and/or activity. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of GSPT1. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of GSPT1 using RNAi technology.

In certain embodiments, the instant specification provides a vector comprising an siRNA or antisense polynucleotide. In other embodiments, the siRNA or antisense polynucleotide inhibits the expression of GSPT1. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In certain embodiments, an antisense nucleic acid sequence expressed by a plasmid vector is used to inhibit GSPT1 protein expression. The antisense expressing vector is used to transfect a mammalian cell or the mammal itself, thereby causing reduced endogenous expression of GSPT1.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

RNA interference molecules that downregulate GSPT1, such as human GSPT1, are commercially available. Non-limiting examples of GSPT1 RNAi molecules include Human GSPT1 siRNA (Catalog No. abx918796) available from Abbexa (Cambridge, UK), GSPT1 Human Pre-designed siRNA Set A (Catalog No. HY-RS05868) available from MedChemExpress (Monmouth Junction, NJ, USA), siRNA IDs 10855, 10949, 11038, 144883, 144884, and 144885 available from ThermoFisher (Waltham, MA) and the like.

### Downregulating GSPT1 Protein Expression by Ribozyme

In some embodiments, the compound that down regulates the activity or expression level of GSPT1 includes a ribosome that inhibits GSPT1 protein expression.

A ribozyme is used to inhibit GSPT1 protein expression. Ribozymes useful for inhibiting the expression of a target molecule may be designed by incorporating target sequences into the basic ribozyme structure which are complementary, for example, to the mRNA sequence encoding GSPT1. Ribozymes are antisense RNAs which have a catalytic site capable of specifically cleaving complementary RNAs. Therefore, ribozymes having sequence complementary to GSPT1 mRNA sequences are capable of downregulating the expression of GSPT1 by reduces the level of GSPT1 mRNA. Ribozymes targeting GSPT1, may be synthesized using commercially available reagents (Applied Biosystems, Inc., Foster City, CA) or they may be genetically expressed from DNA encoding them. In some embodiments, the DNA encoding the ribozymes are incorporated in a vector, which is described in the “Vector” section elsewhere in the instant specification.

### Downregulating GSPT1 Protein Expression by CRISPR Knockout/Knockdown and Other Gene Editing or Expression Modulating Techniques

In some embodiments, the compound that down regulates the activity or expression level of GSPT1 comprises a nucleic acid that down regulates the expression level of GSPT1 by the means of CRISPR knockout.

In some embodiments, the compound down regulates the activity or expression level of GSPT1 comprises a CRISPR/Cas9 system for knocking out GSPT1.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cas 12, Cas 13, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to skin cells, neurons, stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In certain embodiments, the guide RNA comprises a nucleic acid sequence set forth in SEQ ID NOs: 5-9 and described in Table 1. In certain embodiments, the guide RNA consists of a nucleic acid sequence set forth in SEQ ID NOs: 5-9. Tolerable variations of the guide RNA sequence will be known to those of skill in the art. For example, in certain embodiments, the guide RNA comprises a nucleic acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 5-9.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the compound that down regulates the activity or expression level of GSPT1 comprises a nucleic acid that down regulates the expression level of GSPT1 by the means of CRISPR knockdown. CRISPR knockdown includes, but not limited to, CRISPRCas13 knockdown. (See e.g., Mendez-Mancilla et al., Cell Chemical Biology 29, 1-7, 2021 Jul. 27, and Kushawah et al., Dev Cell. 2020 Sep. 28; 54 (6): 805-817. The entireties of which are incorporated herein by reference).

In some embodiments, the present invention includes any other methods for effecting gene knockdown and/editing, which allow for deletion and/or inactivation of GSPT1, such as but not limited to those described in WO 2018/236840 (which is incorporated herein in its entirety by reference).

Downregulating GSPT1 Protein Activity or Expression by Inactivating and/or Sequestering

In some embodiments, the compound that downregulates the activity or expression level of GSPT1 includes a protein that downregulates the activity of GSPT1 by inactivating and/or sequestering GSPT1. In some embodiment, the compound includes a nucleic acid that express the protein that downregulates the activity of GSPT1 by inactivating and/or sequestering GSPT1. In some embodiments, the compound includes an expression vector that express the protein that downregulates the activity of GSPT1 by inactivating and/or sequestering GSPT1 (see “Vector” section for descriptions on vectors).

In some embodiments, the compound that downregulates the expression level of GSPT1 is a trans-dominant negative mutant of GSPT1, and/or a nucleic acid or a vector expressing the trans-dominant negative mutant of GSPT1.

### T Cell Specific Delivery

In some embodiments, the compounds disclosed herein are delivered via a T cell-specific delivery method.

In some embodiments, the compounds disclosed herein are delivered in the form of antibody-drug conjugates (ADCs) in which an antibody specifically targeting T cells is conjugated with the compounds herein.

For example, Parameswaran et al. (Blood (2021) 138 (Supplement 1): 1193) and Zhang et al. (JCI Insight. 2023 Dec. 8; 8 (23): e172914) both describes that conjugating drug molecules on CD6-targeted antibodies was able to deliver the drug molecule specifically into T cells.

Conjugating small molecule inhibitors, such as PROTACs or molecular glues to antibodies is also widely known in the art. Indeed, ADCs including GSPT1 inhibitors (such as degraders) as drugs have also been reported. For example, ORM-5029, ORM-1023, GSPT-001, and ORM-6151/BMS-986497 from Orum Tx are examples of ADCs in which cancer antigen-specific antibodies conjugated are conjugated with degraders of GSPT1.

For another example, the compound disclosed herein can be delivered into T cells by nanoparticles. T cell specific delivery using nanoparticles is described in, for example, Lee et al. (Biomater Res. 2021; 25:44), Cevaal et al. (ACS Nano 2021, 15, 3, 3736-3753), Haist et al. (Front. Immunol., 24 May 2022), Gong et al. (Nature Nanotechnology volume 16, pages25-36 (2021)), and others.

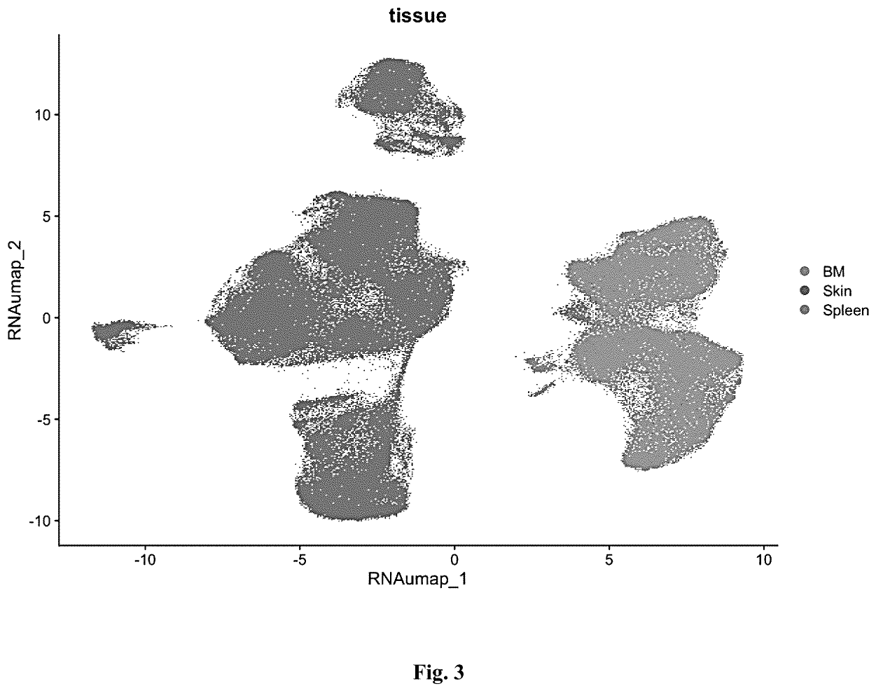

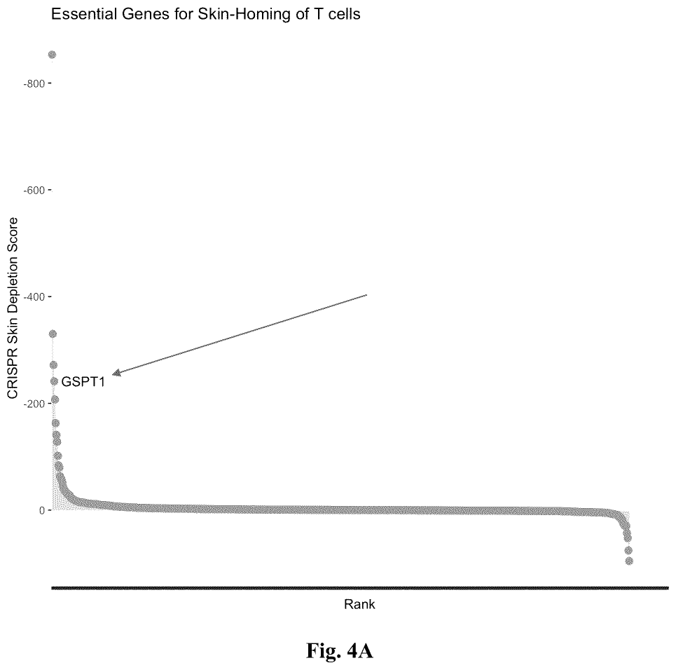

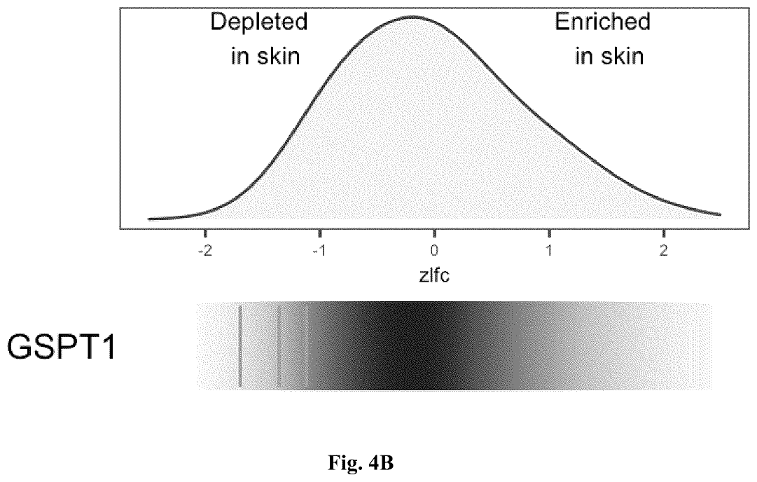

It is also worth noting that, while delivering the compounds disclosed herein can be limited to T cells reside in the skin in accordance with some embodiments, the delivery is not limited to only the skin-homing T cells. Rather, the present study discovered that GSPT1 is highly expressed in skin-homing T cells but is either absent or expressed at low levels in circulating T cells (such as those reside in the bone marrow, spleen or blood, see e.g., FIGS. 3 and 4A-4B). As such, delivering the compounds herein into all T cell types in the body is expected to have relatively limited impact on the circulating T cells.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that that down regulates the activity or expression level of GSPT1 is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that downregulates GSPT1 described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the T cells, especially skin-homing T cells (e.g., TRMs reside in the skin). In some embodiments, the promoter is a T cell-selective promoter or a T cell-specific promoter. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the autoimmune/inflammatory condition includes administering to the subject the effective amount of at least one compound and/or composition contemplated within the disclosure.

In some embodiments, the composition for treating autoimmune/inflammatory condition includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating autoimmune/inflammatory conditions, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In some embodiments, the carrier is a hydrogel. In some embodiments, the composition herein, such as the pharmaceutical composition herein, is formulated as a hydrogel.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of autoimmune/inflammatory conditions in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compounds or compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration. In some embodiments, the compounds or composition herein is administered via topical route.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. patent application Ser. Nos. 20/030,147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the autoimmune/inflammatory condition in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Experimental Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Human Skin Xenograft as a Model for Studying Skin-Homing and Tissue-Resident Memory T Cells (TRM)

The skin presents a challenging environment for T cells due to lower oxygen levels compared to the bloodstream and limited nutrient availability. TRM cells must adapt to this nutrient-poor, hypoxic environment of the skin to maintain long-term survival and functionality. Their resilience is driven by metabolic reprogramming and mTOR-mediated protein synthesis. This adaptation creates an intriguing paradox: TRM require sustained protein synthesis to maintain their tissue-resident identity and function, yet must meet high biosynthetic demands in a nutrient-poor environment. Based on this rigorous scientific foundation, we propose that GSPT1 resolves this paradox by maximizing translation efficiency, ensuring each transcript yields functional protein while minimizing energy expenditure. By enhancing translational fidelity and efficiency, GSPT1 preserves the epigenetic, transcriptional, and proteomic landscapes that underlie the specialized functions of TRM in human skin.

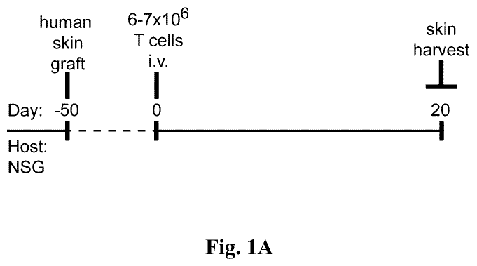

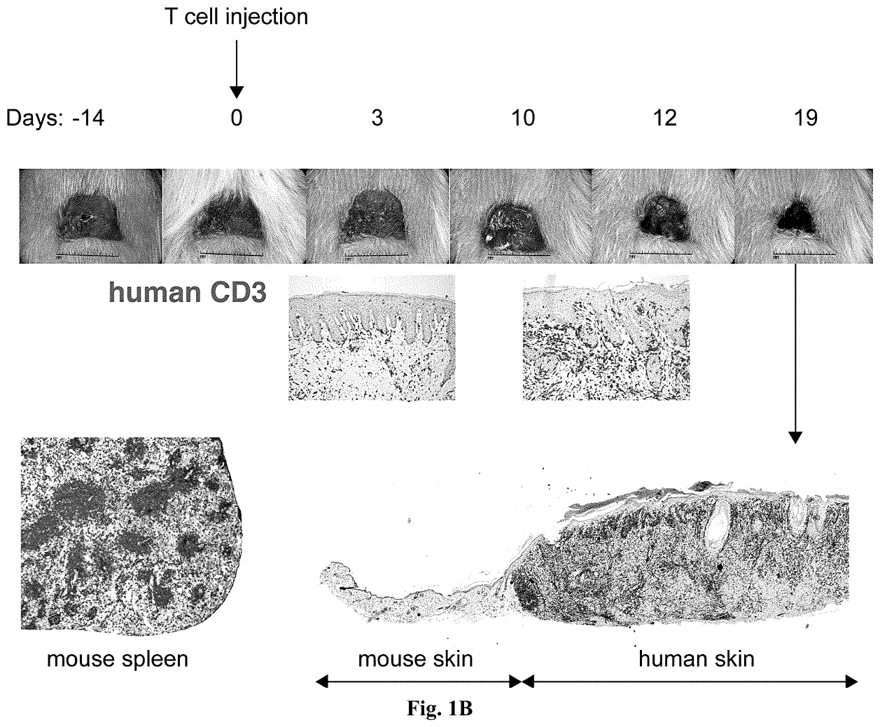

To dissect human TRM adaptations, an advanced human skin xenograft model was employed using immunodeficient NSG also known as NOD.Cg-Prkde^{scid} Il2rg^{tmlWil}/SzJ) mice. Human neonatal foreskin (which lacks intrinsic T cells) is grafted onto NSG mice at day. At day 0, between 6×10⁶ and 7×10⁶ primary human T cells were introduced into the mice intravenously. At day 20, the skin xenografts were harvested (FIG. 1A). Within days, T cells initiate a polyclonal alloreactive (low-affinity) response in the skin (FIG. 1A). The polyclonal, alloreactive nature of this model mimics the activation of autoreactive T cells through the T cell receptor in autoimmune skin diseases and is therefore desirable since autoantigens in these conditions remain poorly defined. Skin inflammation becomes macroscopically and microscopically evident within 1-2 weeks, providing an excellent platform for dissecting T cell entry, exit and tissue adaptation mechanisms.

Notably, a subset of T cells remains circulating and detectable in spleen and bone marrow as human T cells introduced into the mice were detected by staining for human CD3. The human T cells were found to be in human skin graft and differentiate towards tissue-resident memory T cells (TRMs) or remain in compartments such as spleen, blood, or bone marrow. (FIG. 1B), creating a clear fork in the T cell fate decision-either remaining in circulation or committing to the skin TRM lineage. This dual outcome makes the model uniquely suited to study the TRM versus circulating T cell fate decision. The ability to simultaneously track both CD4 and CD8 T cell subsets offers a comprehensive view of tissue residency programs across T cell lineages.

### Example 2: CRISPR/Cas9 Knock-Out Screen Identified GSPT1 as a Target for Inhibiting T Cell Function in the Skin

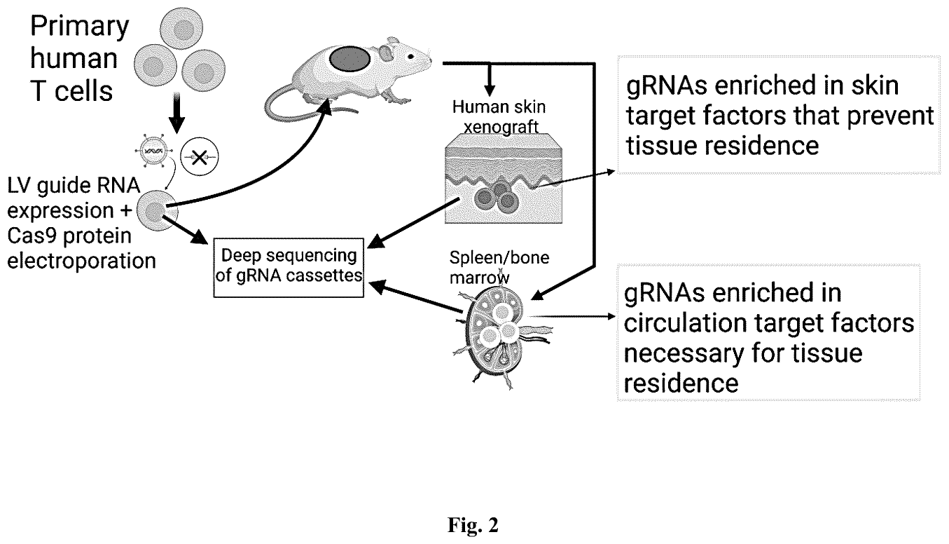

Referring to FIG. 2, the present study carried out a genome-wide in vivo CRISPR/Cas9 knock-out screen of primary human T cells infiltrating human skin xenografts.

The genome-wide CRISPR KO library (known as ‘Brunello’ library) was obtained from Addgene. The library was amplified by electroporation of plasmid DNA into Lucigen's Endura electrocompetent E. coli cells according to the manufacturer's recommendations, grown overnight at 30 degrees Celsius. Endotoxin free plasmid DNA was prepared from the cells using standard isolation methods. Lentiviral particles were produced in HEK 293 by co-transfection of the library plasmid with three additional packaging plasmids and concentrated from tissue culture supernatant after 24 and 48 hours by centrifugation.

To conduct a genome-wide CRISPR KO screen, 150×10⁶ primary human T cells were activated for 24 hours with anti-CD3/anti-CD28 beads before transduction with lentivirus encoding 76,441 gRNAs targeting 19,114 human genes (resulting in a ˜400 fold cell/guide ratio). Transduced cells were be electroporated with Cas9 protein on day 3 after transduction. Transduces T cells expressing gRNAs were selected for by puromycin selection (2.5 micrograms/mL) for three days (day 5-7). Transduction efficiency was determined by incubating decreasing numbers of unselected T cells with identical puromycin concentrations (2.5 micrograms/mL) for 48 hours and compare their viability to T cells that were cultured at the same concentration but not treated with puromycin. All T cell batches demonstrated transduction efficiencies below 20% before selection, confirming that the large majority of T cells expressed only one gRNA.

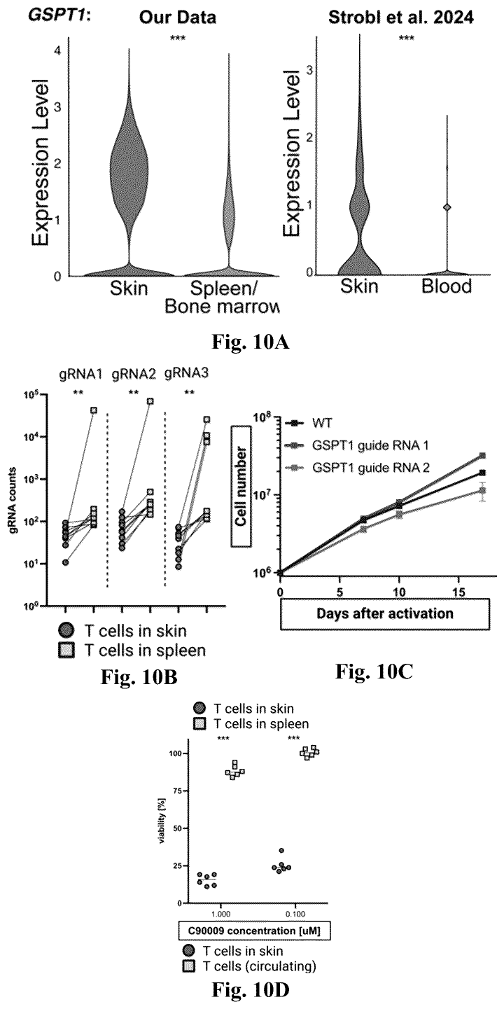

The present study grafted human foreskin samples (which lack resident memory T cells) onto NSG mice and the graft was allowed to heal for 6-7 weeks before mice were injected intravenously with 5-10×10⁶ CRISPR engineered T cells, which are HLA-mismatched to stimulate T cell activation and proliferation after homing to human skin xenografts. The alloreactive nature of this model mimics the activation of autoreactive T cells through the T cell receptor in autoimmune skin diseases and is therefore desirable since autoantigens in these conditions remain poorly defined. As skin inflammation in this model occurs within two weeks, the present study harvested skin, bone marrow and spleen after week 3, isolated genomic DNA and PCR-amplified the gRNA cassette followed by production of a standard Illumina paired-end sequencing library. The read count of individual gRNAs and genes was calculated and compared to the T cell pool prior to infusion and control non-targeting gRNAs. Enrichment and depletion of gRNAs was calculated using the Mageck pipeline comparing bone marrow/spleen with skin. For each gene, the log 2 fold enrichment in skin infiltrating and spleen T cells was be calculated. In this way, the primary human T cells were edited with the CRISPR/Cas9 system and the gRNA library including gRNAs targeting genome-wide genes to knock-out different genes in different T cells. This assay is illustrated in FIG. 10A.

In confirmatory experiments, the present study cloned gRNAs targeting the human PTRPC (CD45) locus into the lentiviral plasmid pJRH051 (obtained from Addgene) or used the lentiviral plasmid pCAT003 that targets the human TRAC locus. Lentiviral particles from these 2 plasmids were produced as described above and primary human T cells transduced with lentivirus encoding the two gRNAs 24 hours after activation. Electroporation with Cas9 protein was performed as above and surface staining of CD45 and CD3 (T cell receptor complex) was performed 10 days after activation. The proportion of edited cells was calculated by dividing the CD45/TRAC positive cells in the edited T cell group by the positive cells in the non-electroporated control group. This percentage was divided by the percent transduced cells, which was obtained by quantifying the proportion of neon positive cells by flow cytometry (neon is a fluorescent marker that is expressed together with the gRNAs). Using example antigens such as CD45 and TRAC, it was confirmed that the edit efficiency was more than 85%. (See FIG. 10B) It was also confirmed that more than 375 T cells were transduced for each of the gRNAs. The edited T cells were then introduced into NSG mice in the manner as described in Example 1.

After IV injection of these T cells into human skin xenograft mice, samples were collected from the skin, bone marrow, and spleen after 25 days. By comparing the frequency of different gene knockouts between skin-resident and circulating T cells (from bone marrow and spleen), genes which were important for T cells to enter and survive in the skin could be identified. Across multiple experiments with different donors (n=9), it was found that when certain genes were knocked out, T cells failed to establish skin residence. By comparing these results with those from the gene expression data allowed for the identification of genes whose expression was increased in TRM, which identified them as part of the natural tissue-adaptation program. Notably, GSPT1 emerged as top hit, whose loss severely impaired T cells' ability to become tissue-resident (FIG. 10C) alongside known TRM regulators (NR4A3, ELL2, FOSL2). Thus, this unbiased, genome-wide loss-of function screen revealed GSPT1 as a previously unknown regulator of tissue residency, providing strong evidence that protein translation termination is an essential component of the human T cell skin adaptation program. See also FIG. 3, which illustrates that T cells in skin and spleen/bone marrow (BM) display distinct expression profiles.

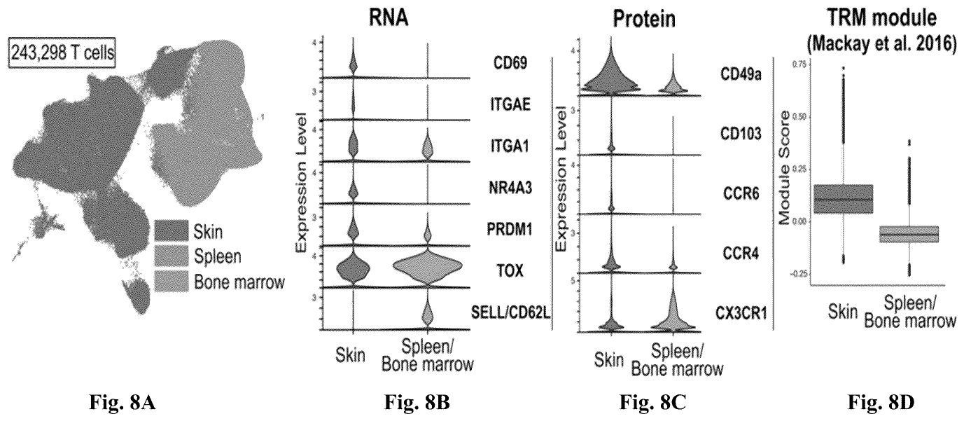

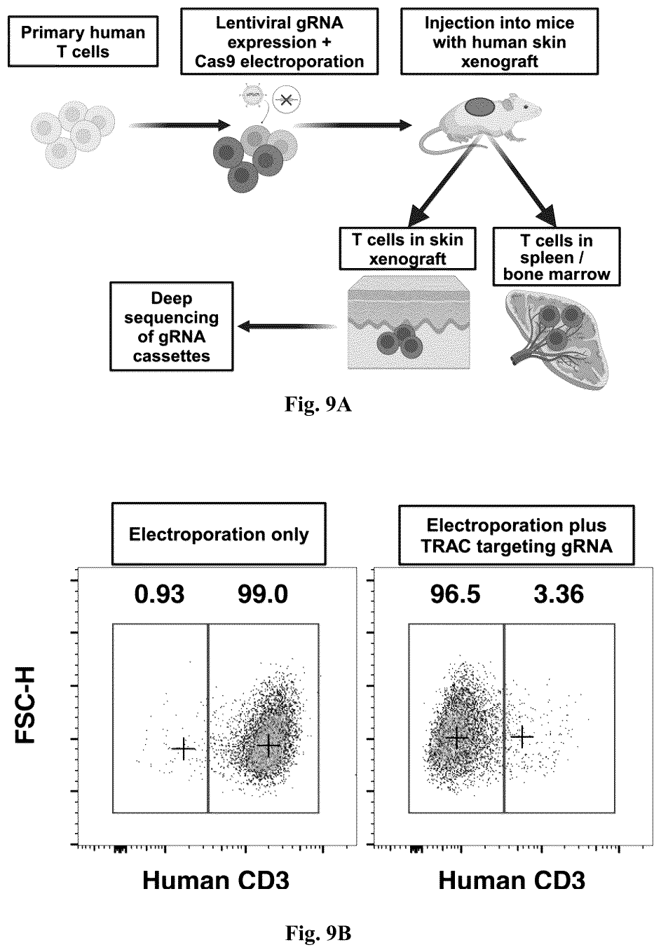

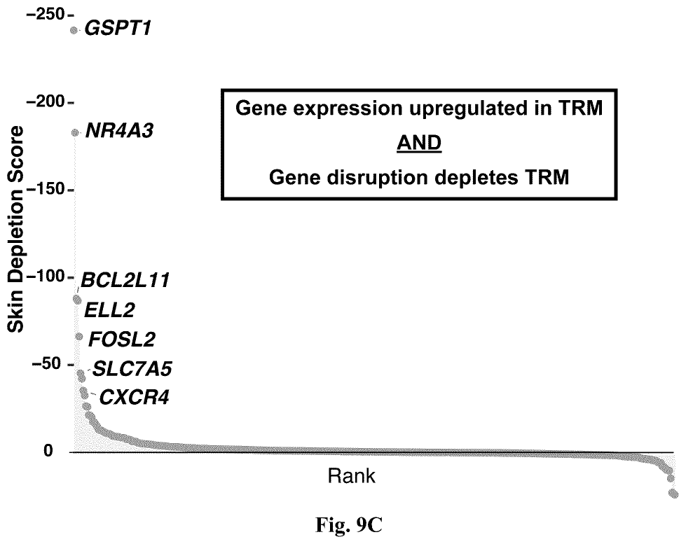

In another study, the skin-homing T cells in the xenograft model were confirmed to become TRM by using split-pool single cell barcoding (Parse Bio) which allows to profile the transcriptome of 1×10⁶ cells at single cell resolution. Three weeks post-injection, T cells were isolated from skin xenografts (CD3/CD45 flow cytometry sorted) and from spleen/bone marrow, then analyzed with combined single-cell RNA-seq and surface protein profiling (CITE-seq46) using a 154-antibody panel. In total, 243,298 T cells (n=4) were profiled. Skin-derived T cells clustered separately from circulating T cells (FIG. 8A), showing elevated expression of canonical TRM genes (CD69, ITGAE, ITGA1, NR4A3, PRDM117,36,47, (FIG. 8B) and lower exhaustion/circulation markers (TOX48, SELL). Surface protein data confirmed increased TRM homing markers (CD103, CD49a, CCR436) and decreased CX3CR1 compared to circulating T cells (FIG. 8C). A module score for 57 established TRM genes from a published, independent dataset49 further verified that most skin T cells (both CD4 and CD8) fulfill the TRM profile, while spleen/bone marrow T cells do not (FIG. 8D). Together, these data demonstrate that skin-infiltrating T cells in this human xenograft model faithfully recapitulate the transcriptome and surface proteome of TRM, making it a powerful tool to dissect human TRM differentiation.

### Example 3: GSPT1 is Upregulated in Skin-Homing T Cells and TRM and Plays a Key Role in Maintaining their Survival and Function

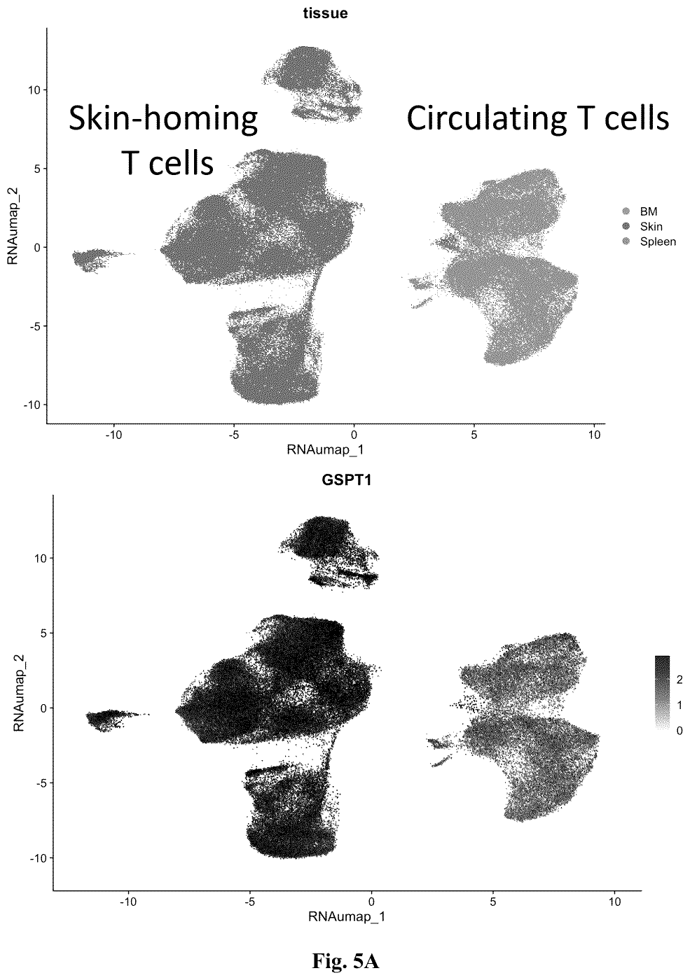

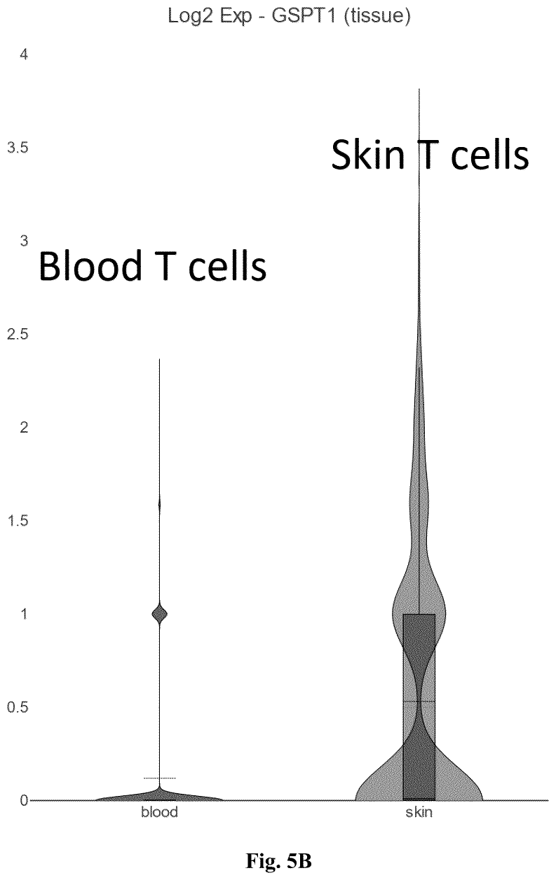

Referring to FIGS. 5A-5B, the present study discovered that skin T cells substantially increase their GSPT1 expression, which demonstrates an important role of GSPT1 in T cell tissue adaptation.

Referring to FIG. 5A, skin T cells demonstrated increased expression of GSPT1 compared to circulating T cells

Referring to FIG. 5B, study of single cell expression of skin and circulating T cells from human GVHD patients demonstrated that skin T cells have higher expression of GSPT1 when compared to circulating T cells.

In the experiments, 5×10⁶ T cells were injected intravenously into skin xenograft bearing mice as described above. Three weeks later, skin grafts, spleens and bone marrow were isolated. Skin tissue was digested with liberase TL (0.25 g/ml) for 2 hours at 37° C. T cells from spleen, bone marrow and skin were sorted by flow cytometric sorting (BD ARIA) by sorting live, CD45+ cells. Following flow sorting, cells were stained with the Biolegend Totalseq-A universal human cocktail according to the manufacturer's recommendation further processed according to the Parse Biosciences single cell TCR workflow and sequences on an Illumina Novaseq 6000 sequencer. Single cell analysis was performed with the Parse Bio split-pipe pipeline and further analyzed with Seurat.

GSPT1 expression in TRM was further confiemd in an additional, independent human graft-versus-host disease cohort (FIG. 10A). Further, it was determined that all detectable GSPT1-targeting gRNAs in these screens were consistently enriched in circulating T cells compared to skin TRM, confirming the robustness of these screening results (FIG. 10B). Importantly, GSPT1 deficiency following CRISPR editing did not affect circulating T cell proliferation in vitro (FIG. 10C), indicating that GSPT1-deficiency is not essential for circulating T cell expansion after activation.

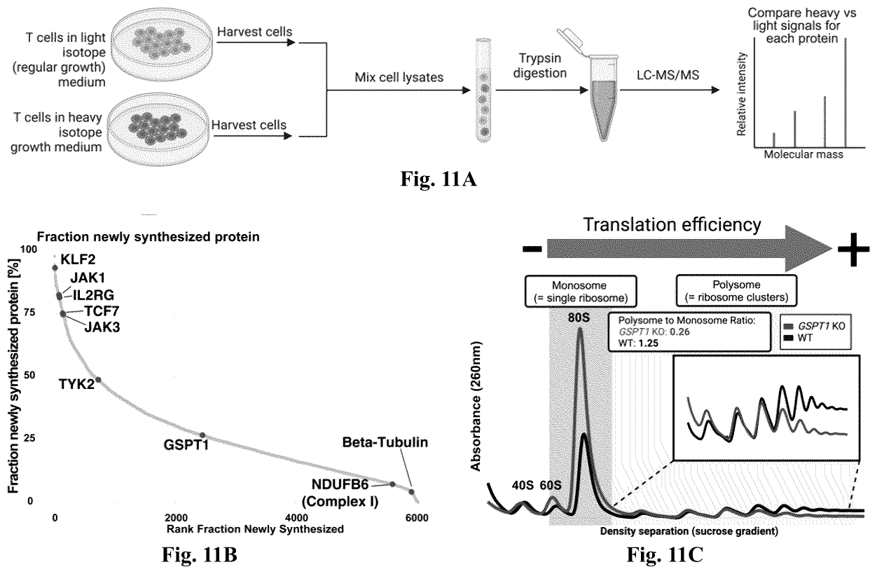

To establish the importance of GSPT1 expression on the TRM translatome and overall translation efficiency, SILAC (Stable Isotope Labeling by Amino Acids in Culture) experiments were conducted using primary human T cells (isolated from peripheral blood) from two donors (FIG. 11A). This method precisely tracks protein abundance changes by comparing heavy-isotope (Lys-8, Arg-10) vs. normal (“light”) amino acid incorporation. Unstimulated T cells were cultured 24 hours in media containing either “heavy” or “light” amino acids, then mixed and digested for high-resolution LC-MS/MS on a Thermo Orbitrap Astral (Wistar Proteomics Core, see letter of support from Dr. Tang). Using data-independent acquisition (DIA) and MSFragger-DIA (FDR <1%), protein turnover was accurately quantified (FIG. 11B). The observed patterns matched previously published data for naive T cells58, confirming our approach. We noted rapid turnover for critical transcription factors (KLF2, TCF7) and for therapeutic targets such as JAK1/3, TYK2, and IL2RG (>50-75% newly synthesized within 24 hours), explaining why inhibitors often have limited durability. By contrast, housekeeping proteins (B-tubulin) and metabolic components (NDUFB6) had slower turnover. Importantly, GSPT1 also exhibited slow turnover, suggesting that its therapeutic targeting might achieve more sustained effects compared to current approaches targeting JAKs or cytokine receptors. Previous studies have shown that circulating memory T cells maintain higher baseline translation rates compared to naive T cells, enabling their rapid recall responses. As we identified GSPT1 as essential for TRM, understanding these cell's unique translational landscape becomes especially relevant for deciphering how “translation efficiency” is maintained under resource scarcity. These data demonstrate how GSPT1 loss affects protein synthesis rates in TRM vs. circulating T cells.

To complement the SILAC studies (which measures ongoing protein synthesis over time-FIG. 11C), a polysome profiling study was performed. This technique provides a snapshot of how many ribosomes are actively translating mRNA. T cells (isolated from peripheral blood of healthy donors) were briefly treated with cycloheximide to “freeze” ribosomes on mRNA transcripts, then fractionated via density gradient centrifugation to separate mRNAs by ribosome load. This directly informs “translation efficiency” since higher polysome loading often correlates with higher protein output per bound mRNA, while fewer polysomes suggest diminished ribosome engagement (workflow illustrated in FIG. 11A). This method is especially relevant for GSPT1, which facilitates translation termination and ribosome recycling. The approach was validated in activated human circulating T cells, achieving 98% GSPT1 knockout without affecting circulating T cell survival, aligning with clinical data showing 90% GSPT1 depletion is tolerable in blood T cells. Notably, the polysome: monosome ratio fell five-fold in GSPT1-knockout cells, indicating substantially reduced ribosome engagement and thus reduced translation efficiency, even though these cells still proliferated normally in culture (FIG. 11C). This disconnect suggests that GSPT1-driven efficient termination is especially critical under the nutrient-poor, hypoxic conditions of skin, where TRM must optimize protein output. Thus, GSPT1 may be dispensable for survival in well-nourished environments (e.g., blood) but becomes essential for high-fidelity protein synthesis in resource-limited skin niches. Overall, these data confirm (I) our capacity to perform polysome profiling in primary T cells and (II) why GSPT1 is upregulated and required in TRM, setting the stage to compare translation dynamics in TRM vs. circulating T cells and to investigate how GSPT1 deficiency disrupts translation efficiency in human skin.

### Example 4: GSPT1 Depletion Dose-Dependently Induced T Cell Apoptosis and Resulted in the Inability of T Cells to Persist in the Skin Niche

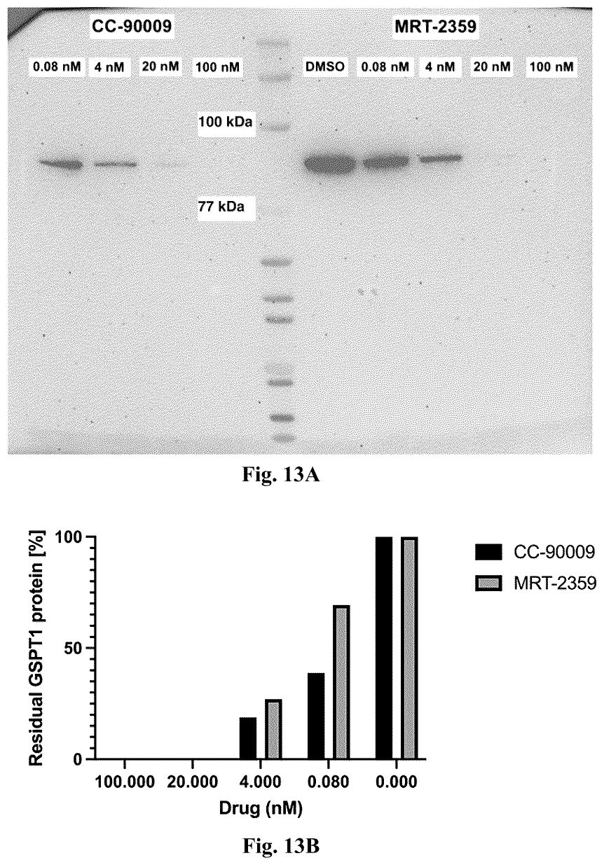

Given that GSPT1-degrading small-molecules are already advancing through early-phase trials for cancer, it was tested whether targeting GSPT1 could selectively affect TRM in vitro. TRM were isolated from healthy human skin (n=6) and their sensitivity was to the GSPT1-degrading drug CC-90009 with that of human blood T cells. After 72 hours of treatment, skin TRM showed reduced viability in response to GSPT1 depletion than their circulating counterparts (FIG. 10D). Thus, GSPT1 is upregulated and essential for TRM. In a similar study, another small molecule GSPT1 degrader, MRT-2359, was used to treat peripheral blood T cells from normal human donors for 24 hours in vitro at various concentrations. Western blot analysis demonstrated that, like CC-90009, MRT-2359 treatment also resulted in a dose-dependent decrease in GSPT1 expression (FIG. 13A). These data were quantified in FIG. 13B, which shows that both CC-90009 and MRT-2359 cause significant dose-dependent loss of GSPT1 protein in treated T cells.

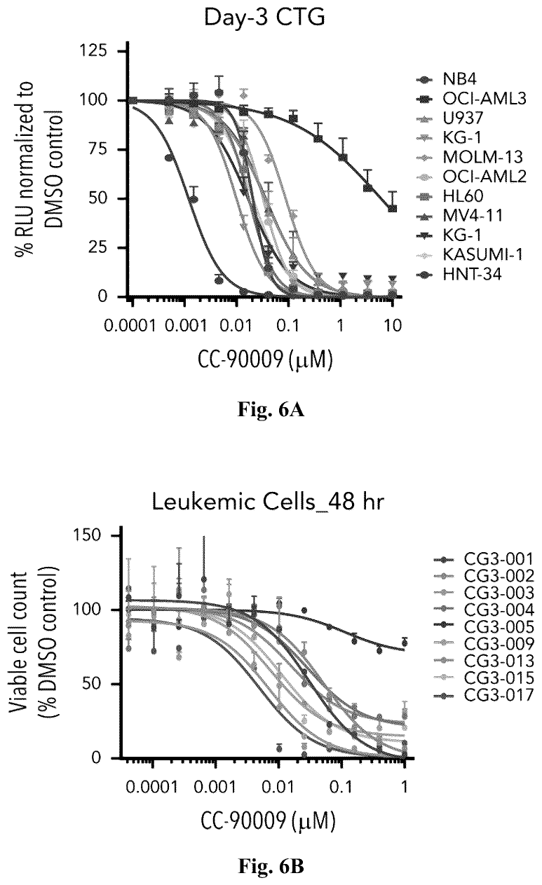

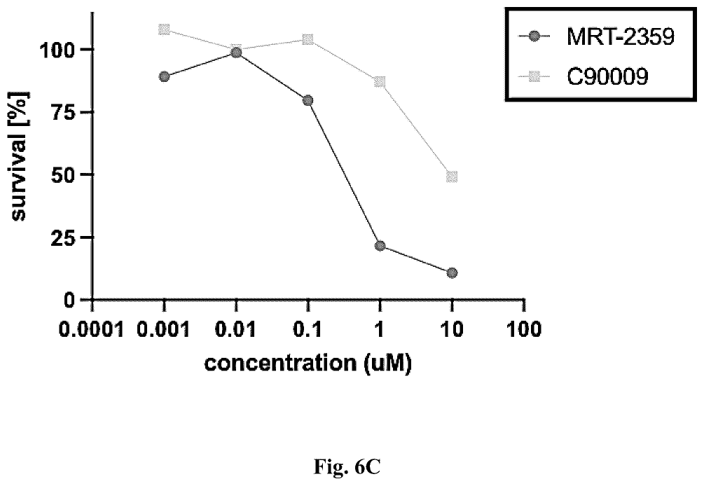

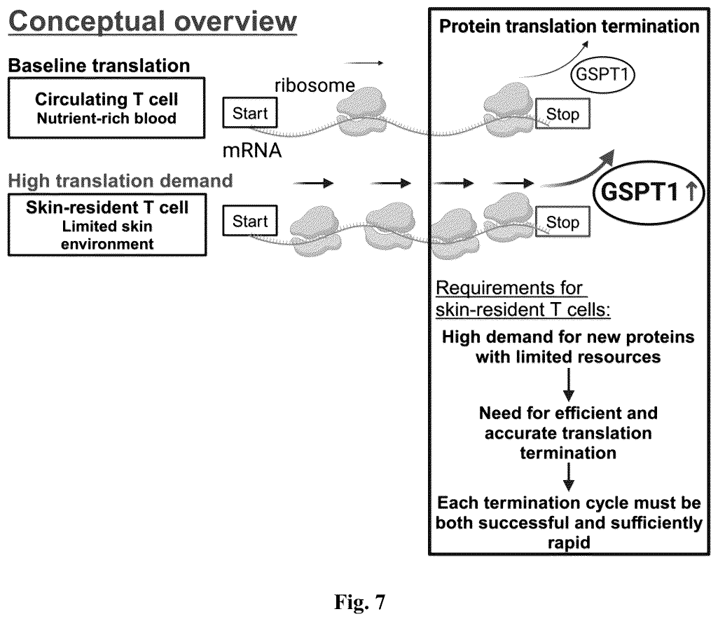

Referring to FIGS. 6A-6C, depletion of GSPT1 with GSPT1 inhibitors resulted in apoptosis in T cells. In FIGS. 6A and 6B, treating acute myeloid leukemia (AML) cells (FIG. 6A) and primary leukemia cells (FIG. 6B) with the GSPT-1 degrader CC-90009 resulted in dose-dependent apoptosis of the cells. In FIG. 6C, treating primary human T cells with the GSPT-1 degraders CC-90009 and MRT-2359 also resulted in does apoptosis of the cells. It is worth noting that, in FIG. 6C, the dosages of CC-90009 required to achieve the same level of T cell apoptosis in the primary T cells (i.e., circulating T cells) were dramatically increased in comparison to the dosage required in the T cells of FIGS. 6A-6B, demonstrating the resistance of circulating T cells to GSPT1 depletion.

GSPT1 depletion was further confirmed to cause the inability of T cells to persist in the skin niche. Referring to FIG. 4B, T cells expressing GSPT1 depleting gRNAs (i.e. deficient in these genes) are depleted from the skin.

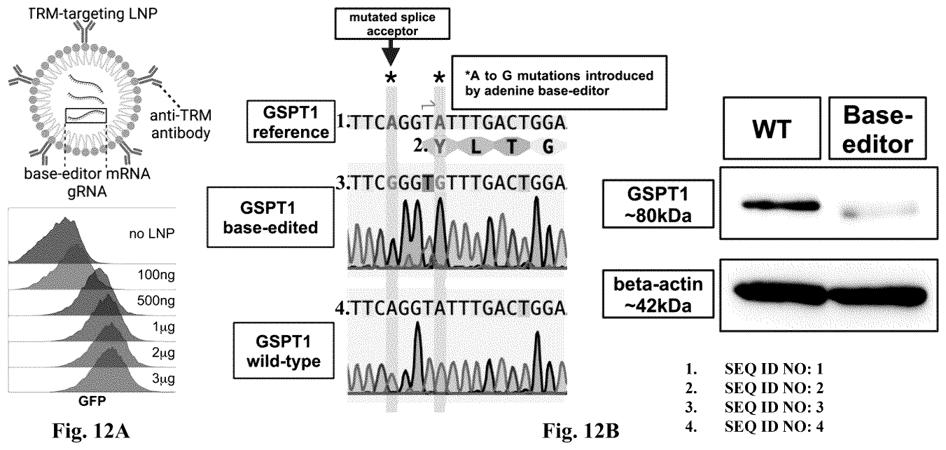

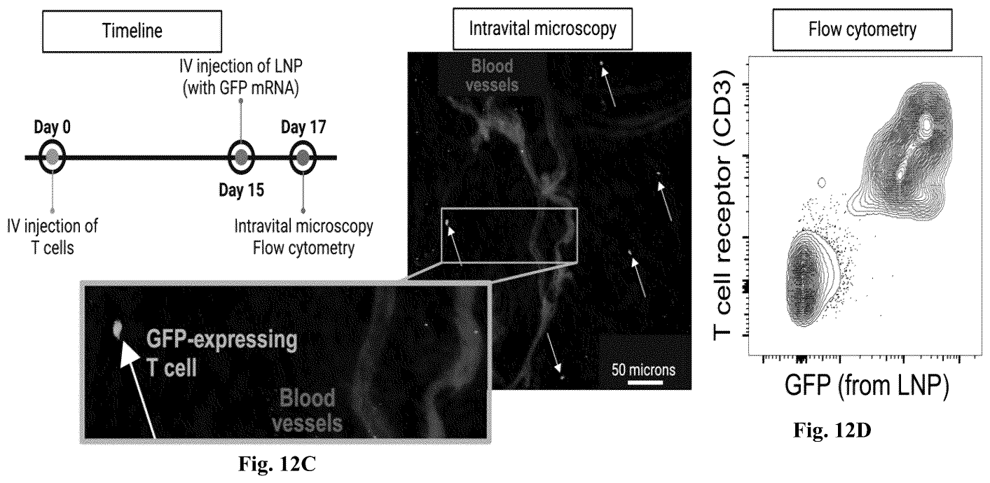

In another set of studies, a lipid nanoparticle (LNP) based system was developed to selectively target GSPT1 in T cells by using CD5 as a pan-T cell marker (though any TRM marker could be substituted; FIG. 12A). LNPs self-assemble from a mixture of ionizable and structural lipids; the ionizable lipids are positively charged at acidic pH for efficient nucleic acid packaging but remain neutral in circulation. Anti-CD5 antibodies were conjugated to the LNP surface, then a microfluidic mixer was used to form uniformly sized particles. Size conformity was confirmed by dynamic light scattering. LNP generated by this platform were then validated in vitro by loading CD5-targeted LNPs with GFP mRNA and then co-incubating them with human donor T cells. Flow cytometric analysis demonstrated a clear LNP dose-dependent GFP expression in T cells, confirming high efficiency of mRNA delivery (FIG. 12A, bottom). The targeted LNPs can carry mRNA or CRISPR components (base editors+guide RNAs) for genetic modification in T cells, potentially enabling selective GSPT1 disruption in TRM while sparing circulating T cells. A follow-up study utilized an adenine base editor (ABE8), which enables precise A·T to G·C conversions without double-strand breaks, using guide RNAs targeting GSPT1 splice site acceptor sequences. Following electroporation of base editor mRNA and guide RNA into T cells, we achieved near-complete GSPT1 knockout as confirmed by sequencing and Western blot (FIG. 12B). Sequences for three of the guide RNAs from the knockout CRISPR screen and two of the guide RNAs used with the base editors in these studies are listed in Table 1. In vivo, after allowing T cells to infiltrate human skin xenografts for 15 days, lug of CD5-targeted LNP-GFP was adminstered by IV injection along with fluorescently-labeled anti-CD31 IgG to visualize blood vessels. Two days later, 2-photon microscopy revealed robust GFP⁺ T cells in the graft, and flow cytometry confirmed nearly all CD3⁺ cells expressed GFP (FIGS. 12C, 12D). These demonstrated that targeted LNPs were able to deliver gene-targeting cargo to skin-resident T cells in vivo.

TABLE 1
Guide RNA sequences

| SEQ ID | | |
|---|---|---|
| NO: | Name | Sequence |
| 5 | Base Editor guide | TTTCAGGTATTTGACTGGAA |
| | 1 targeting GSPT1 | |
| 6 | Base Editor guide | TTATTTCAGGTACTTGTCTT |
| | 2 targeting GSPT1 | |
| 7 | KO guide from | CCAGCGGGAGAACCTCAGCG |
| | CRISPR screen 1 | |
| 8 | KO guide from | AATCCCAAAACCTAAGTCTG |
| | CRISPR screen 2 | |
| 9 | KO guide from | AATGTCTCCAGAAGAATCAT |
| | CRISPR screen 3 | |

### Enumerated Embodiments

In some aspects, the present invention is directed to the following non-limiting embodiments, the numbering of which is not to be construed as designating levels of importance.

Embodiment 1 provides a method of reducing skin-homing T cells or tissue-resident memory T cells (TRM) in a skin tissue, comprising contacting the skin-homing T cells or TRM with an effective amount of a composition that downregulates an expression level and/or an activity of GSPT1.

Embodiment 2 provides the method of embodiment 1, wherein the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

Embodiment 3 provides the method of embodiment 1, wherein the composition is delivered by a T cell-specific delivery method.

Embodiment 4 provides the method of embodiment 1, wherein the composition comprises a lipid nanoparticle (LNP) that is conjugated to an antibody targeting a T cell specific surface antigen.

Embodiment 5 provides the method of embodiment 4, wherein the T cell specific surface antigen is CD5.

Embodiment 6 provides the method of embodiment 1, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

Embodiment 7 provides the method of embodiment 1, wherein the composition comprises a molecular degrader that links GSPT1 to a ubiquitin ligase.

Embodiment 8 provides the method of embodiment 1, wherein the composition comprises a small molecule inhibitor of GSPT1, and wherein the small molecule inhibitor of GSPT1 comprises at least one selected form the group consisting of SP-320T, DuVIVI,

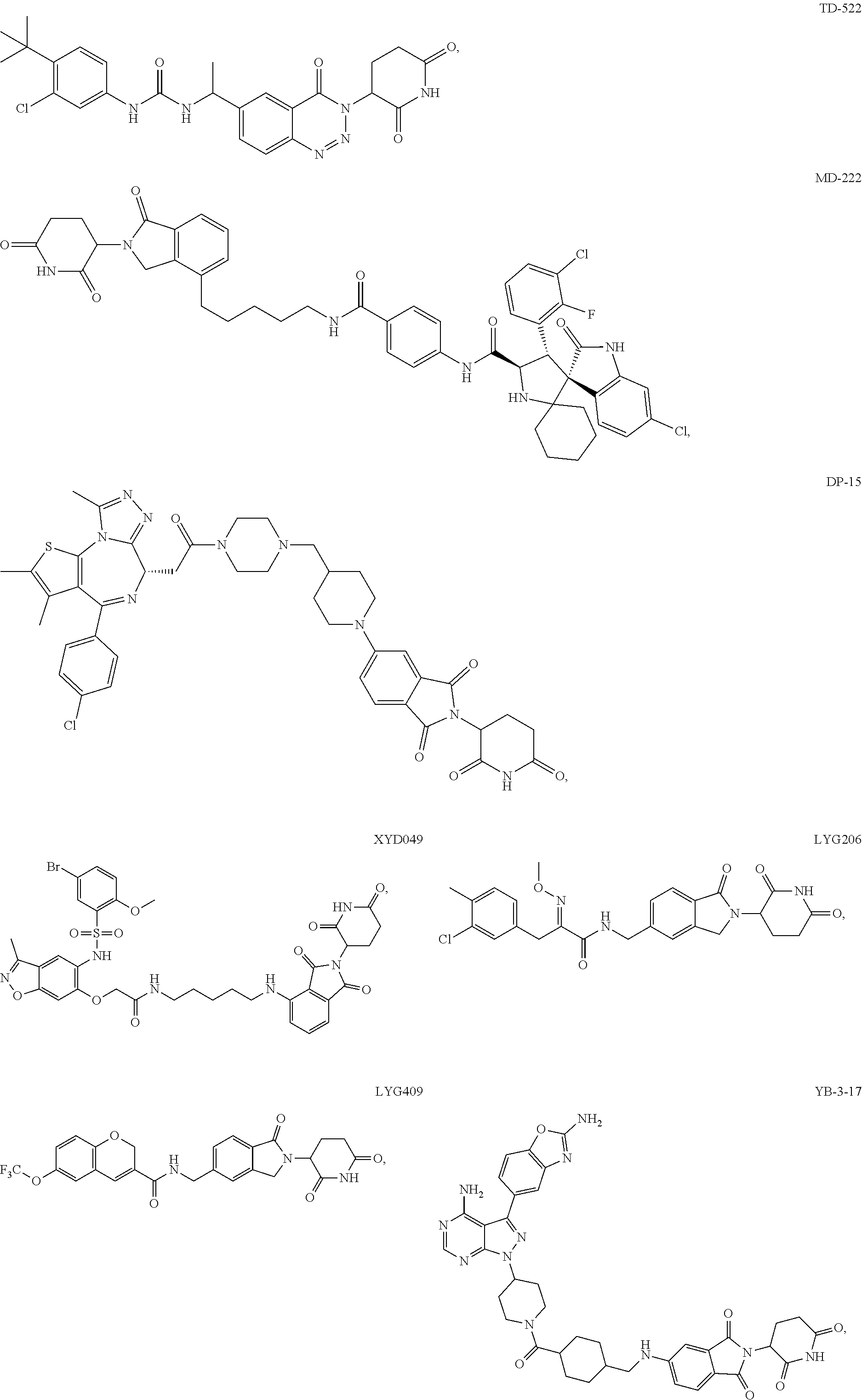

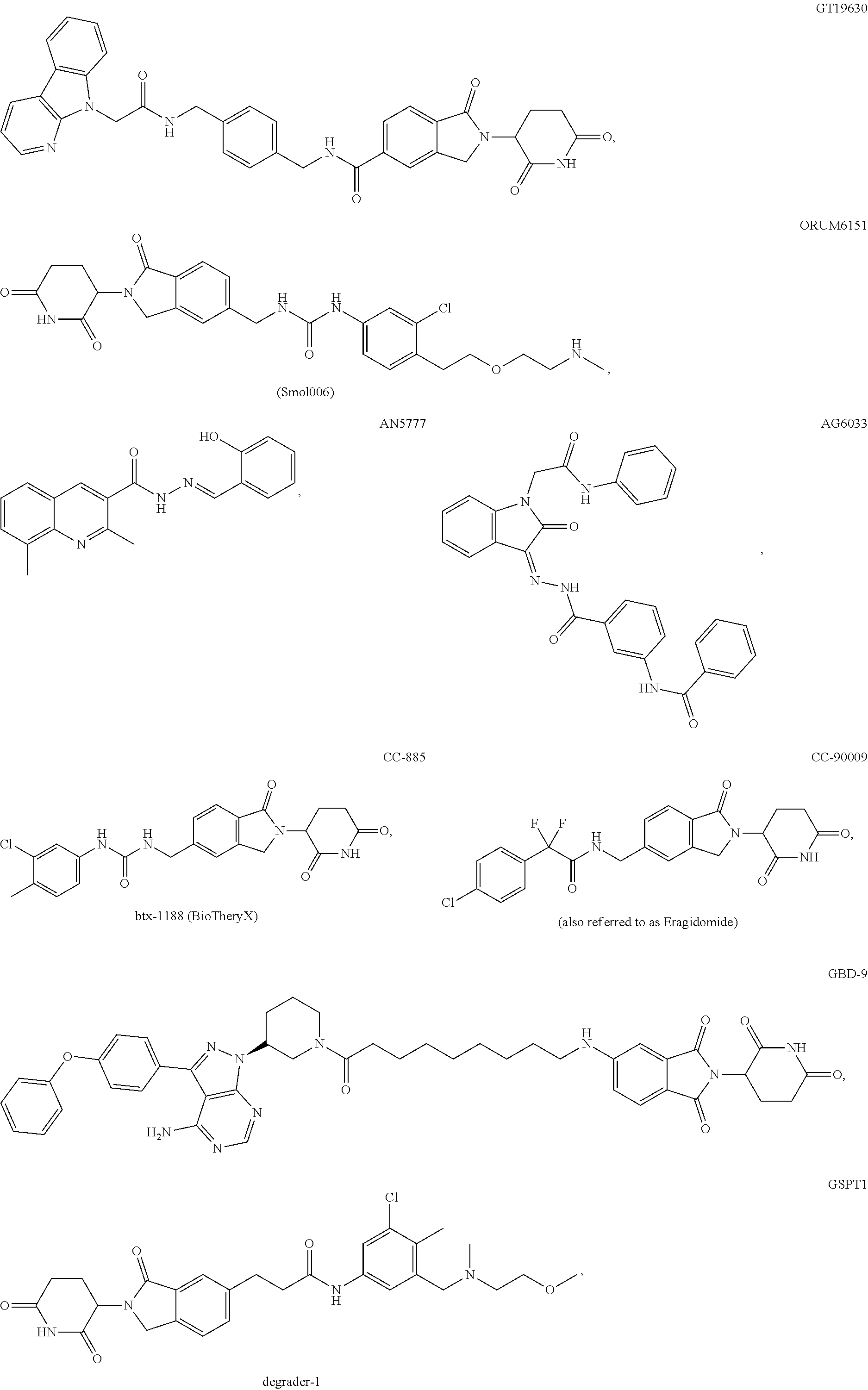

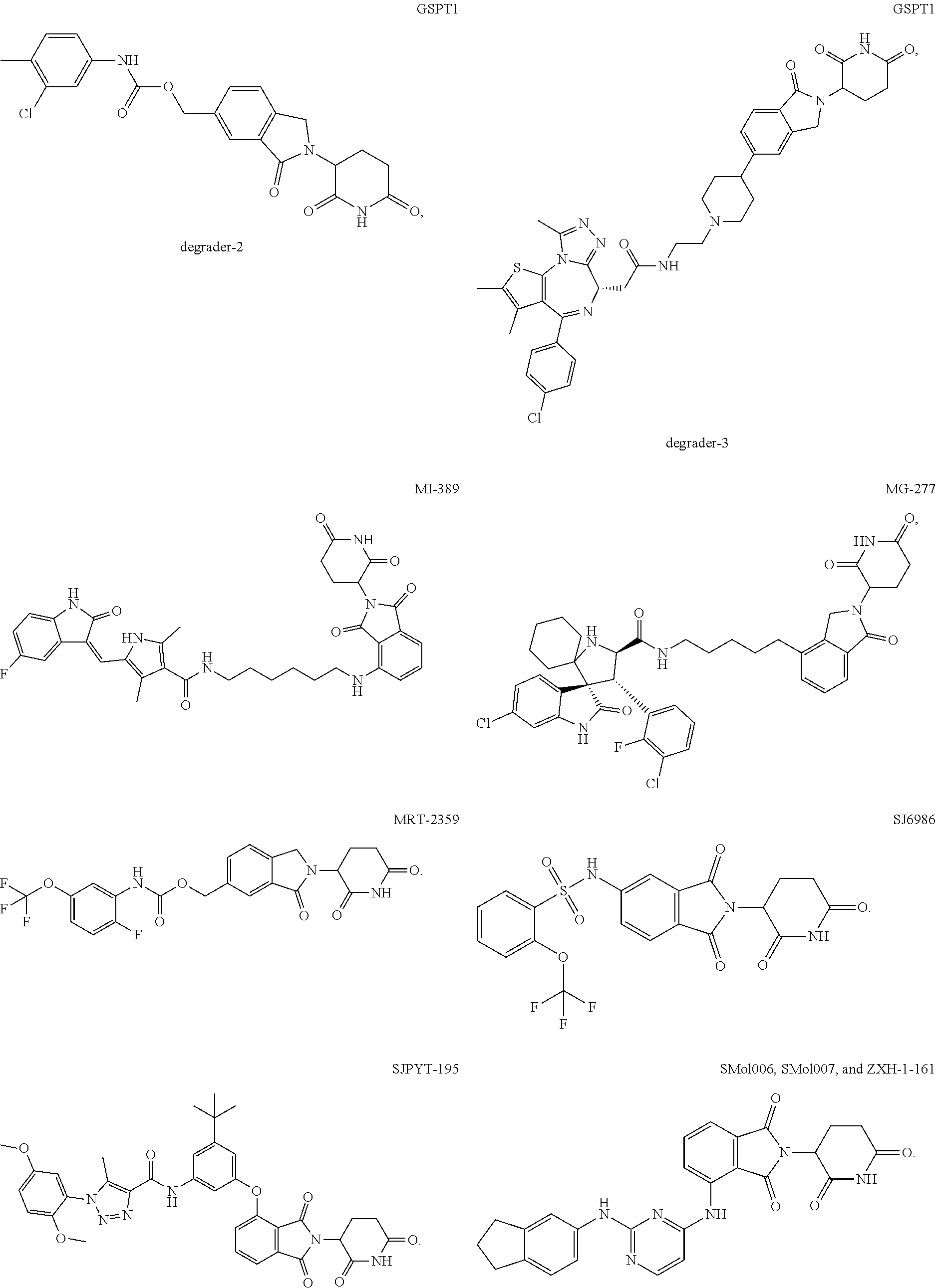

Embodiment 9 provides the method of embodiment 1, wherein administering an effective amount of the composition causes apoptosis in the TRM cells in the skin tissue.

Embodiment 10 provides the method of embodiment 1, wherein at least one of the following applies:
(i) the skin tissue is an isolated skin tissue; or
(ii) the skin tissue is in a subject.

Embodiment 11 provides the method of embodiment 1, wherein the skin tissue is in a subject, and wherein the subject is a mammal, optionally a human.

Embodiment 12 provides a method of selectively depleting T cells in the skin, comprising:
contacting T cells in the skin with a composition that downregulates an expression level and/or an activity of GSPT1.

Embodiment 13 provides the method of embodiment 12, wherein the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

Embodiment 14 provides the method of embodiment 12, wherein the composition is delivered by a T cell-specific delivery method.

Embodiment 15 provides the method of embodiment 12, wherein the composition comprises a lipid nanoparticle, wherein the lipid nanoparticle is conjugated to an antibody targeting a T cell specific surface antigen.

Embodiment 16 provides the method of embodiment 15, wherein the T cell specific surface antigen is CD5.

Embodiment 17 provides the method of embodiment 12, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

Embodiment 18 provides the method of embodiment 12, wherein the composition comprises a molecular degrader that links GSPT1 to a ubiquitin ligase.

Embodiment 19 provides the method of embodiment 12, wherein the composition comprises a small molecule inhibitor of GSPT1, and wherein the small molecule inhibitor of GSPT1 comprises at least one compound selected form the group consisting of SP-3204, CDG0501,

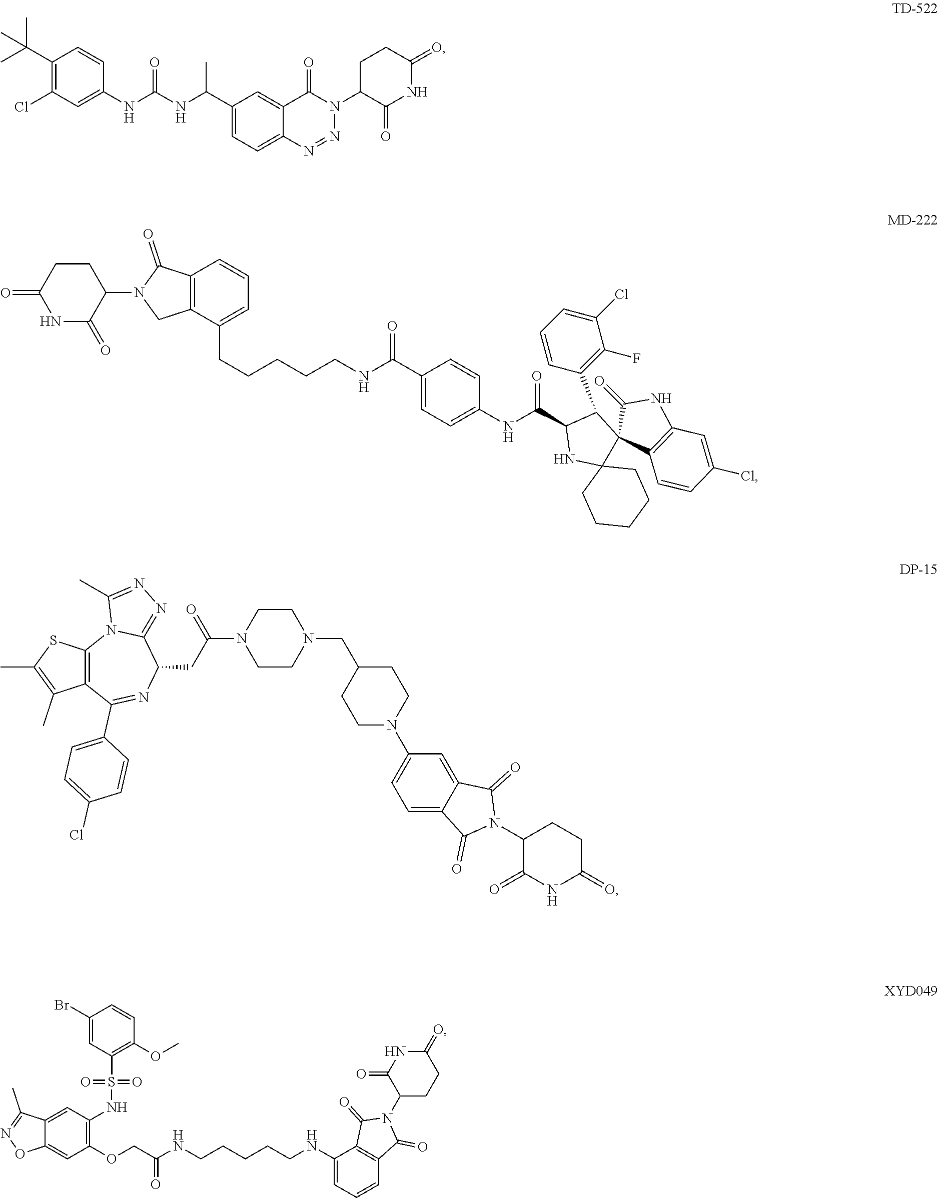 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O]

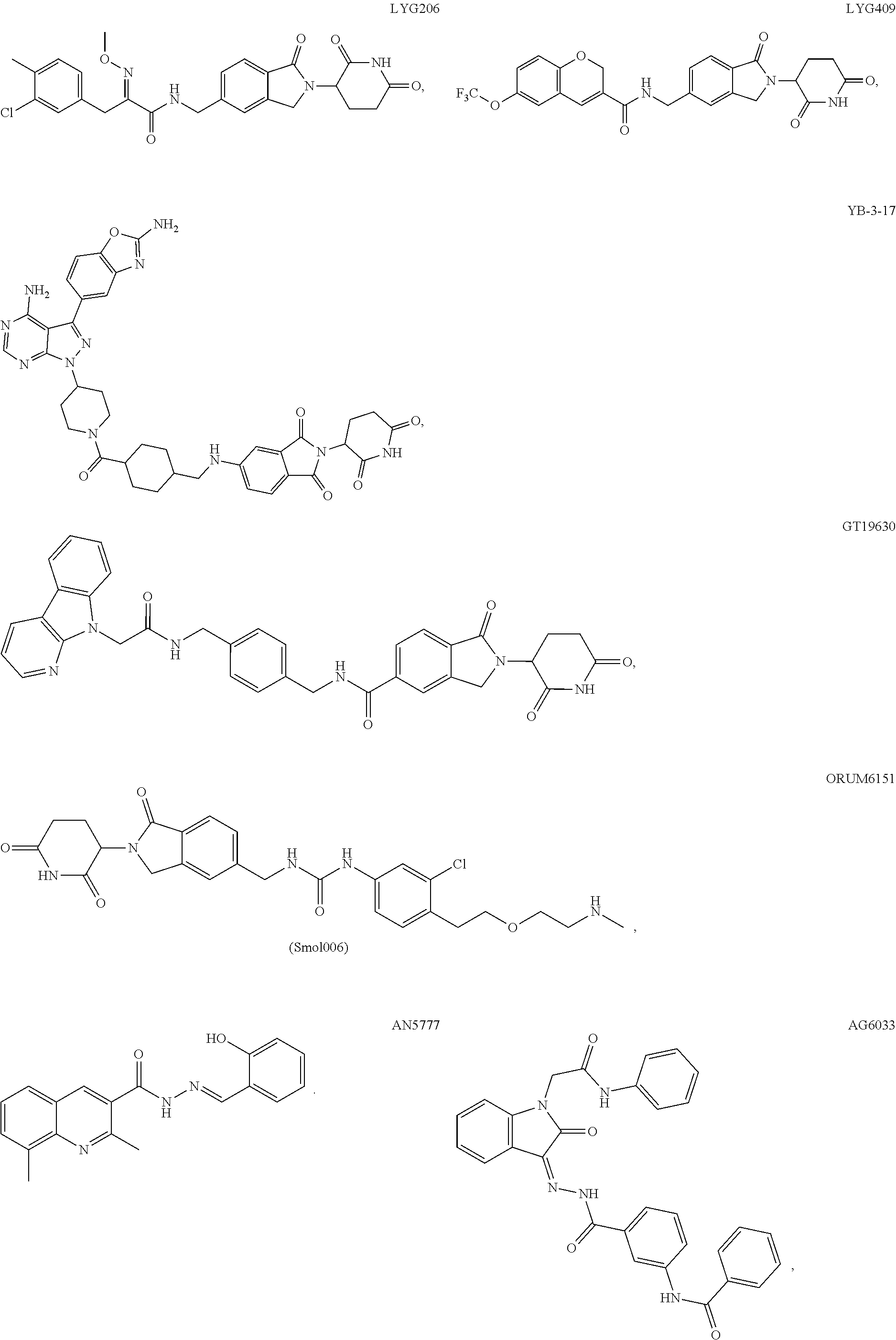

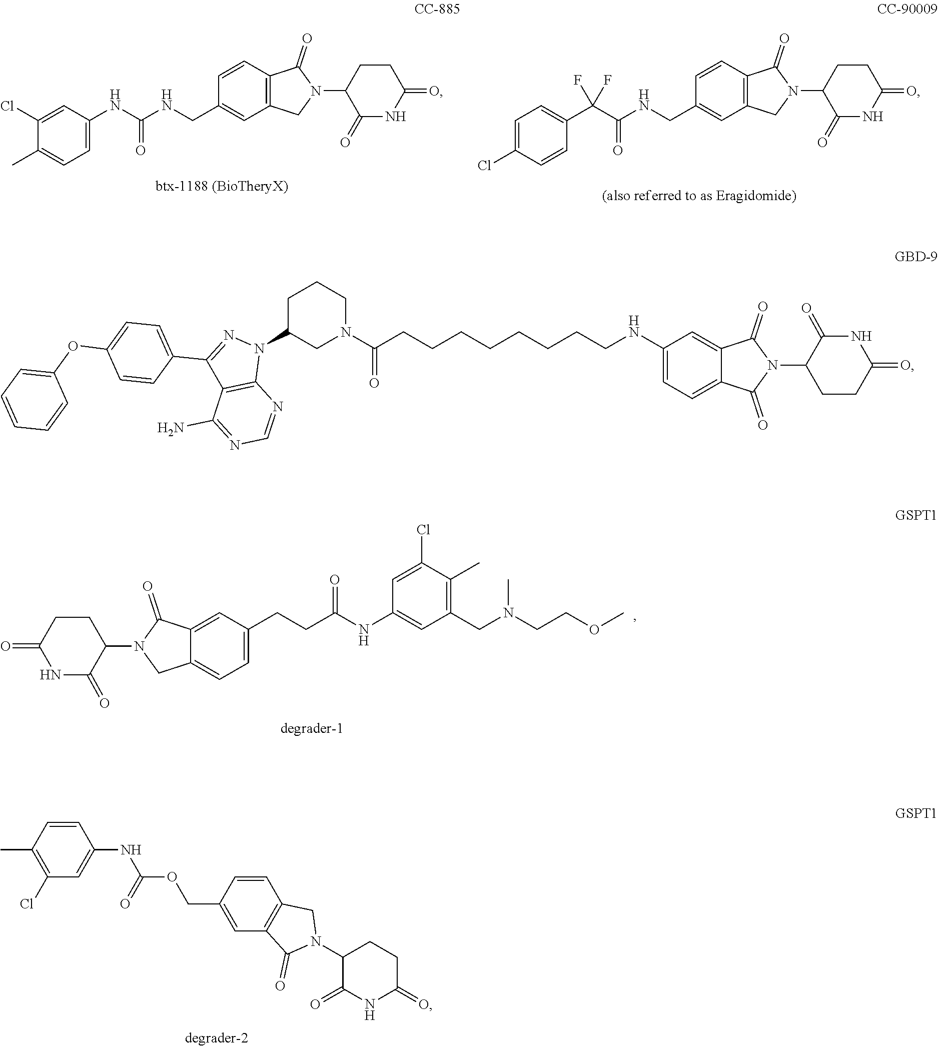

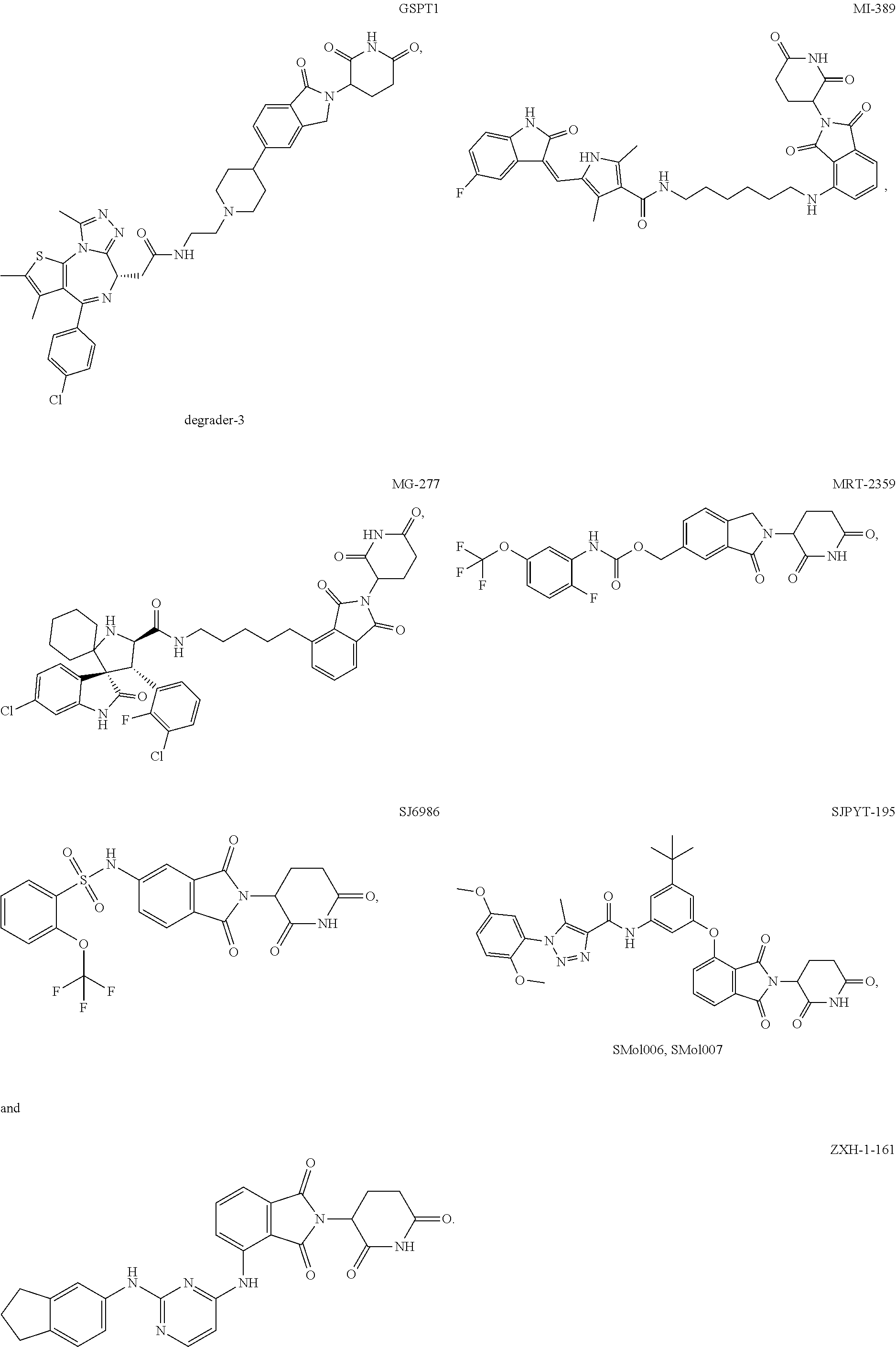

Embodiment 20 provides the method of embodiment 12, wherein administering an effective amount of the compound causes apoptosis in the TRM cells in the skin tissue.

Embodiment 21 provides the method of any one of embodiment 12, wherein at least one of the following applies:
(i) the skin tissue is an isolated skin tissue; or
(ii) the skin tissue is in a subject.

Embodiment 22 provides the method of embodiment 12, wherein the skin tissue is in a subject, and wherein the subject is a mammal, optionally a human.

Embodiment 23 provides a method of treating, ameliorating and/or preventing an autoimmune or inflammatory disease or disorder in a skin in a subject in need thereof, comprising:
administering to the subject an effective amount of a composition that downregulates an expression level and/or an activity of GSPT1.

Embodiment 24 provides the method of embodiment 23, wherein the autoimmune or inflammatory disease or disorder in the skin is alopecia areata, graft-versus-host disease (GVHD), lichen planus, lupus erythematosus, scleroderma (optionally localized forms such as morphea), psoriasis and related conditions such as psoriatic arthritis, Stevens-Johnson syndrome, urticarial vasculitis, vitiligo, atopic dermatitis (eczema), or bullous pemphigoid.

Embodiment 25 provides the method of embodiment 23, wherein the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

Embodiment 26 provides the method of embodiment 23, wherein the composition is delivered by a T cell-specific delivery method.

Embodiment 27 provides the method of embodiment 23, wherein the compound further comprises a lipid nanoparticle, wherein the lipid nanoparticle is conjugated to an antibody targeting a T cell specific surface antigen.

Embodiment 28 provides the method of embodiment 27, wherein the T cell specific surface antigen is CD5.

Embodiment 29 provides the method of embodiment 25, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

Embodiment 30 provides the method of embodiment 23, wherein the composition comprises a molecular degrader that links GSPT1 to a ubiquitin ligase.

Embodiment 31 provides the method of embodiment 23, wherein the composition comprises a small molecule inhibitor of GSPT1, and wherein the small molecule inhibitor of GSPT1 comprises at least one selected form the group consisting of SP-3204, CDG0501,

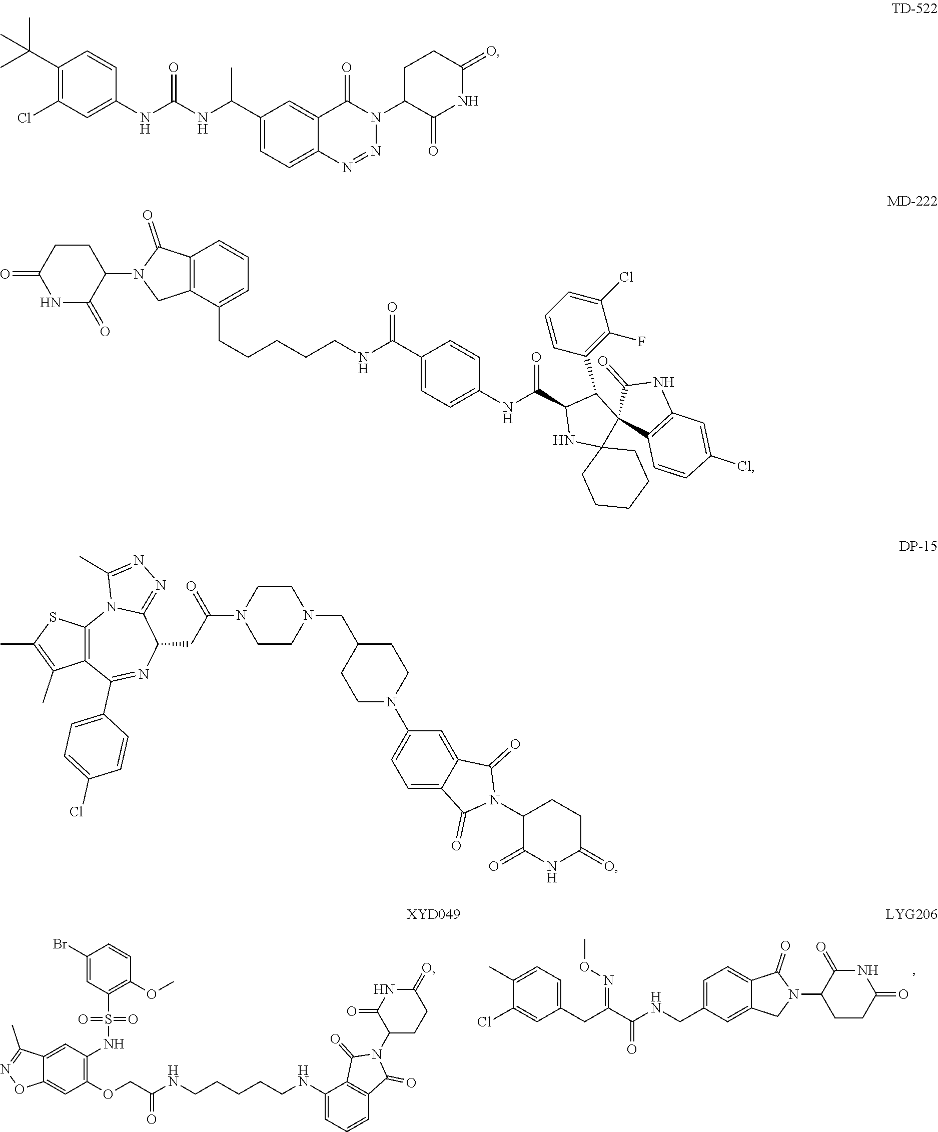 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O ; CO/N=C(\Cc1ccc(C)c(Cl)c1)C(=O)NCc1ccc2c(c1)CN(C1CCC(=O)NC1=O)C2=O]

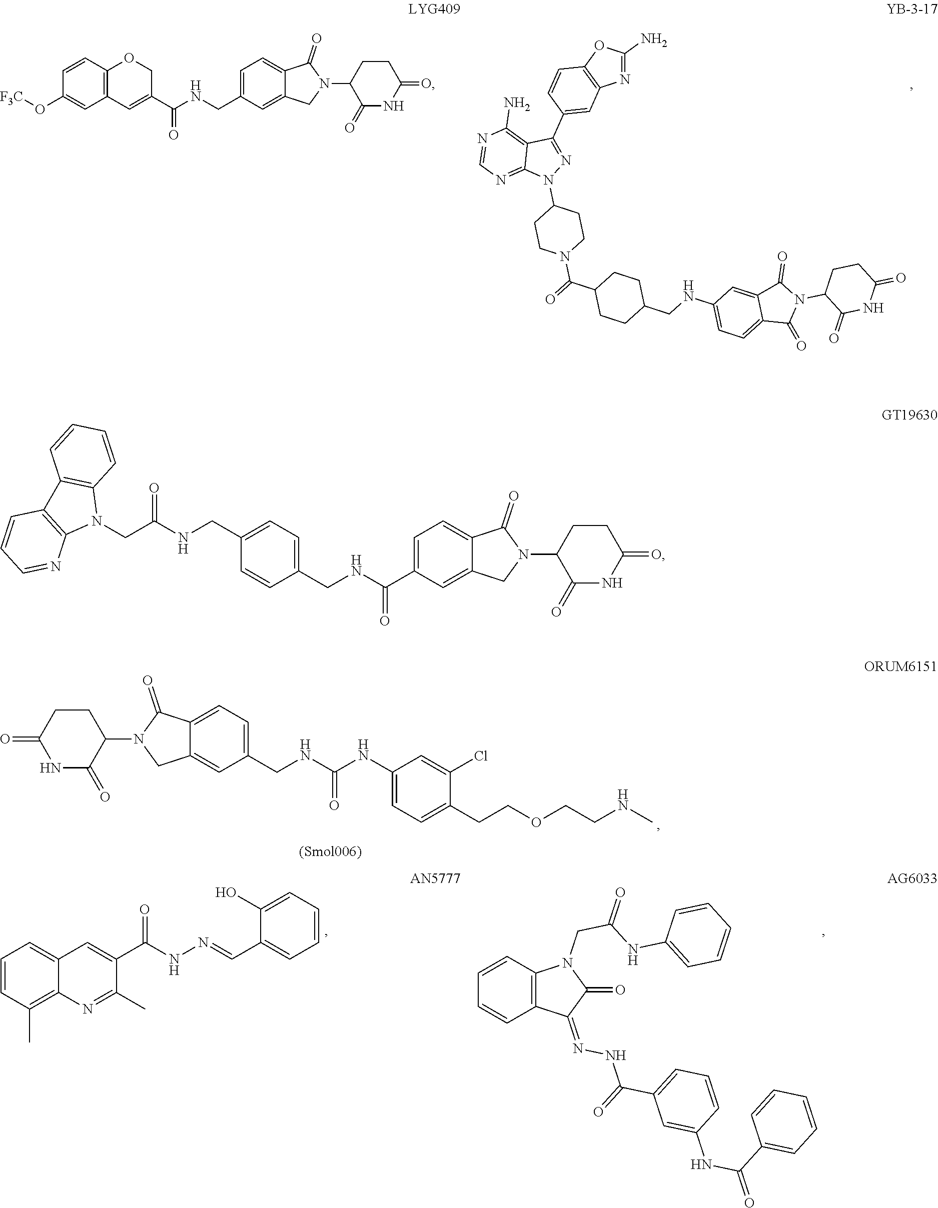 [SMILES: O=C1CCC(N2Cc3cc(CNC(=O)C4=Cc5cc(OC(F)(F)F)ccc5OC4)ccc3C2=O)C(=O)N1 ; Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl ; Cc1nc2c(C)cccc2cc1C(=O)N/N=C/c1ccccc1O ; O=C(CN1C(=O)/C(=N\NC(=O)c2cccc(NC(=O)c3ccccc3)c2)c2ccccc21)Nc1ccccc1]

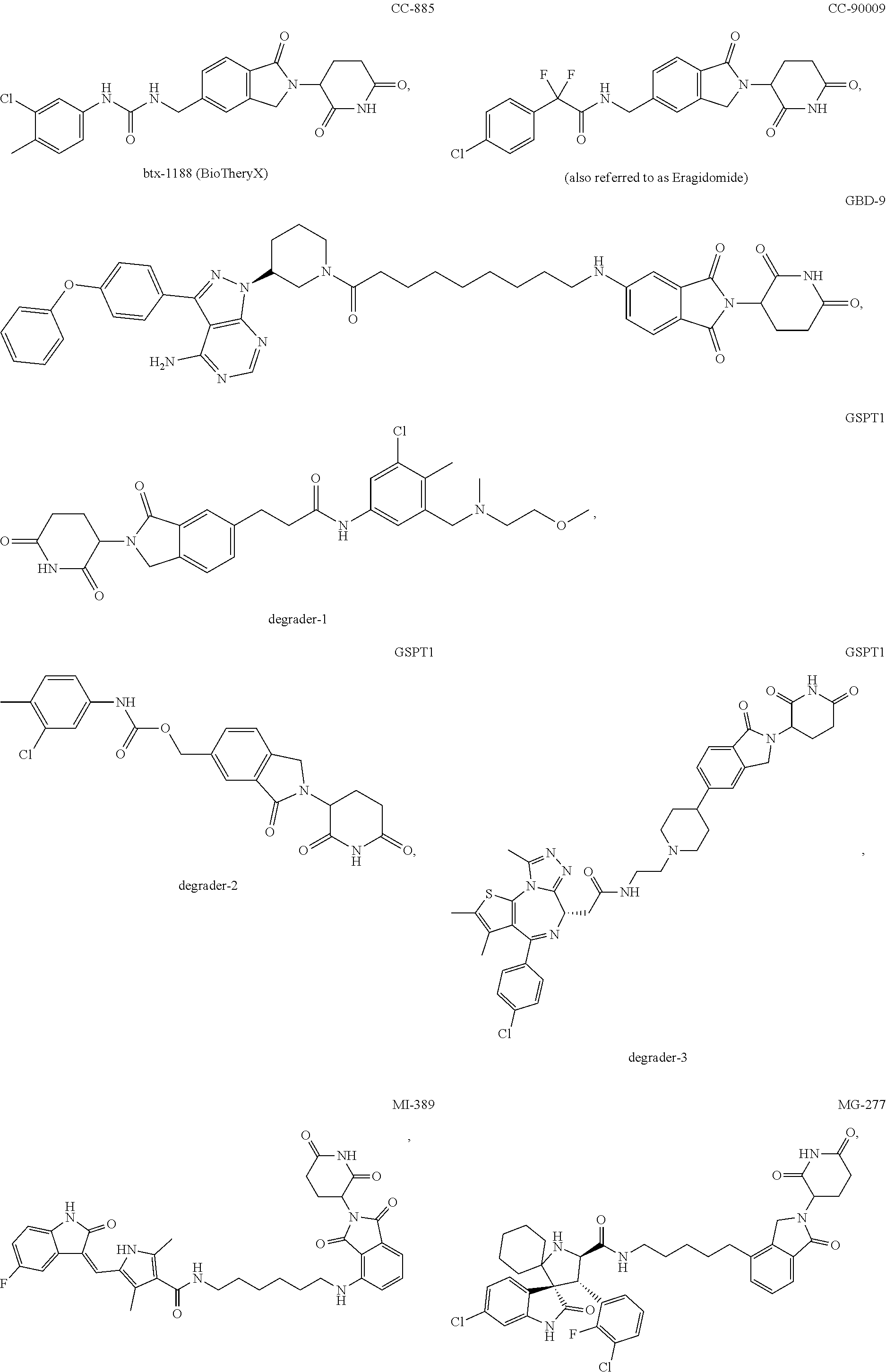

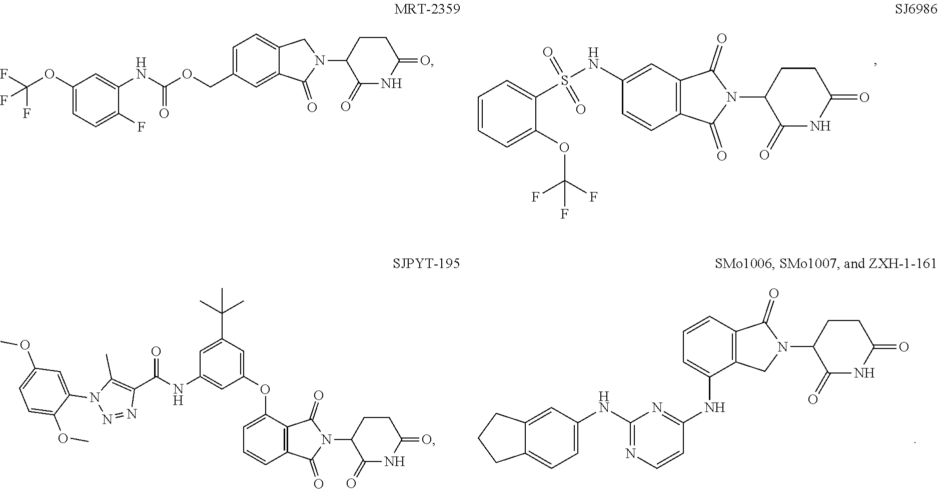 [SMILES: O=C1CCC(N2Cc3ccc(COC(=O)Nc4cc(OC(F)(F)F)ccc4F)cc3C2=O)C(=O)N1 ; O=C1CCC(N2C(=O)c3ccc(NS(=O)(=O)c4ccccc4OC(F)(F)F)cc3C2=O)C(=O)N1 ; COc1ccc(OC)c(-n2nnc(C(=O)Nc3cc(Oc4cccc5c4C(=O)N(C4CCC(=O)NC4=O)C5=O)cc(C(C)(C)C)c3)c2C)c1 ; O=C1CCC(N2Cc3c(Nc4ccnc(Nc5ccc6c(c5)CCC6)n4)cccc3C2=O)C(=O)N1]

Embodiment 32 provides the method of embodiment 23, wherein administering the composition causes apoptosis in skin-homing T cells.

Embodiment 33 provides the method of embodiment 23, wherein administering the composition causes apoptosis in the skin tissue resident memory T cells (TRMs).

Embodiment 34 provides the method of embodiment 23, wherein the composition is a pharmaceutical composition.

Embodiment 35 provides the method of embodiment 23, wherein the composition is administered topically, orally, or parentally.

Embodiment 36 provides the method of embodiment 23, wherein the subject is a mammal, optionally a human.

Embodiment 37 provides a pharmaceutical composition, comprising:
a compound that downregulates an expression level and/or an activity of GSPT1; and
a pharmaceutically acceptable carrier,
wherein the pharmaceutical composition is formulated as a topical formulation.

Embodiment 38 provides a method for treating, ameliorating, and/or preventing an autoimmune or inflammatory disease or disorder in a skin in a subject in need thereof, the method comprising administering an effective amount of the pharmaceutical composition comprising:
a compound that downregulates an expression level and/or an activity of GSPT1; and
a pharmaceutically acceptable carrier,
wherein the pharmaceutical composition is formulated as a topical formulation.

Embodiment 39 provides the method of embodiment 38, wherein the autoimmune or inflammatory disease or disorder is selected from the group consisting of alopecia areata, graft-versus-host disease (GVHD), lichen planus, lupus erythematosus, scleroderma, localized scleroderma such as morphea, psoriasis, psoriasis-related conditions such as psoriatic arthritis, Stevens-Johnson syndrome, urticarial vasculitis, vitiligo, atopic dermatitis (eczema), and bullous pemphigoid.

Embodiment 40 provides the method of embodiment 38, wherein the compound comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

Embodiment 41 provides the method of embodiment 38, wherein the composition is formulated for a T cell-specific delivery method.

Embodiment 42 provides the method of embodiment 38, wherein composition comprises a lipid nanoparticle (LNP) conjugated to an antibody targeting a T cell specific surface antigen.

Embodiment 43 provides the method of embodiment 42, wherein the T cell specific surface antigen is CD5.

Embodiment 44 provides the method of embodiment 40, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

Embodiment 45 provides the method of embodiments 40, wherein the compound comprises a molecular degrader that links GSPT1 to a ubiquitin ligase.

Embodiment 46 provides the method of embodiment 40, wherein the compound comprises a small molecule inhibitor of GSPT1, and wherein the small molecule inhibitor of GSPT1 comprises at least one selected form the group consisting of SP-3204, CDG0501,

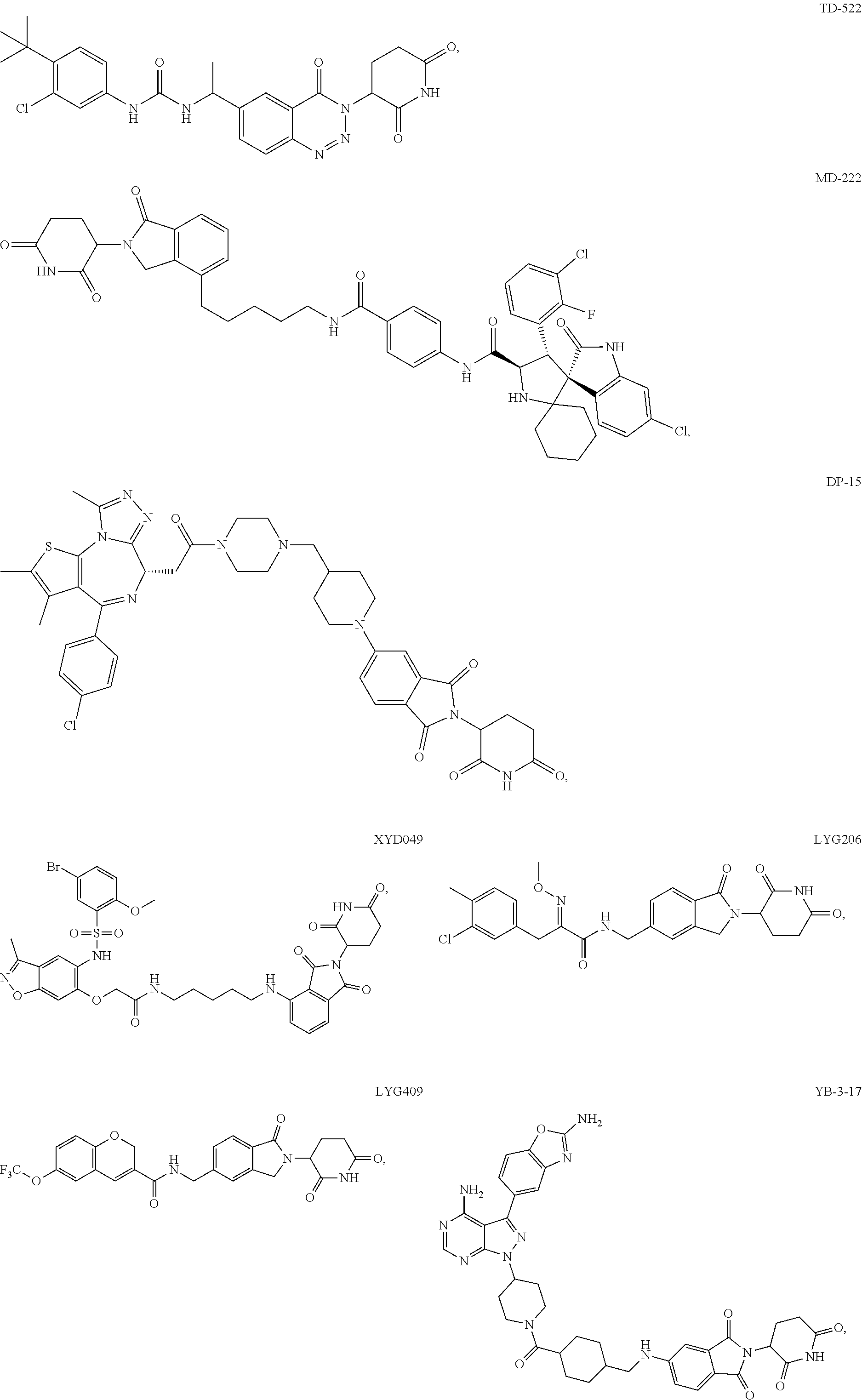

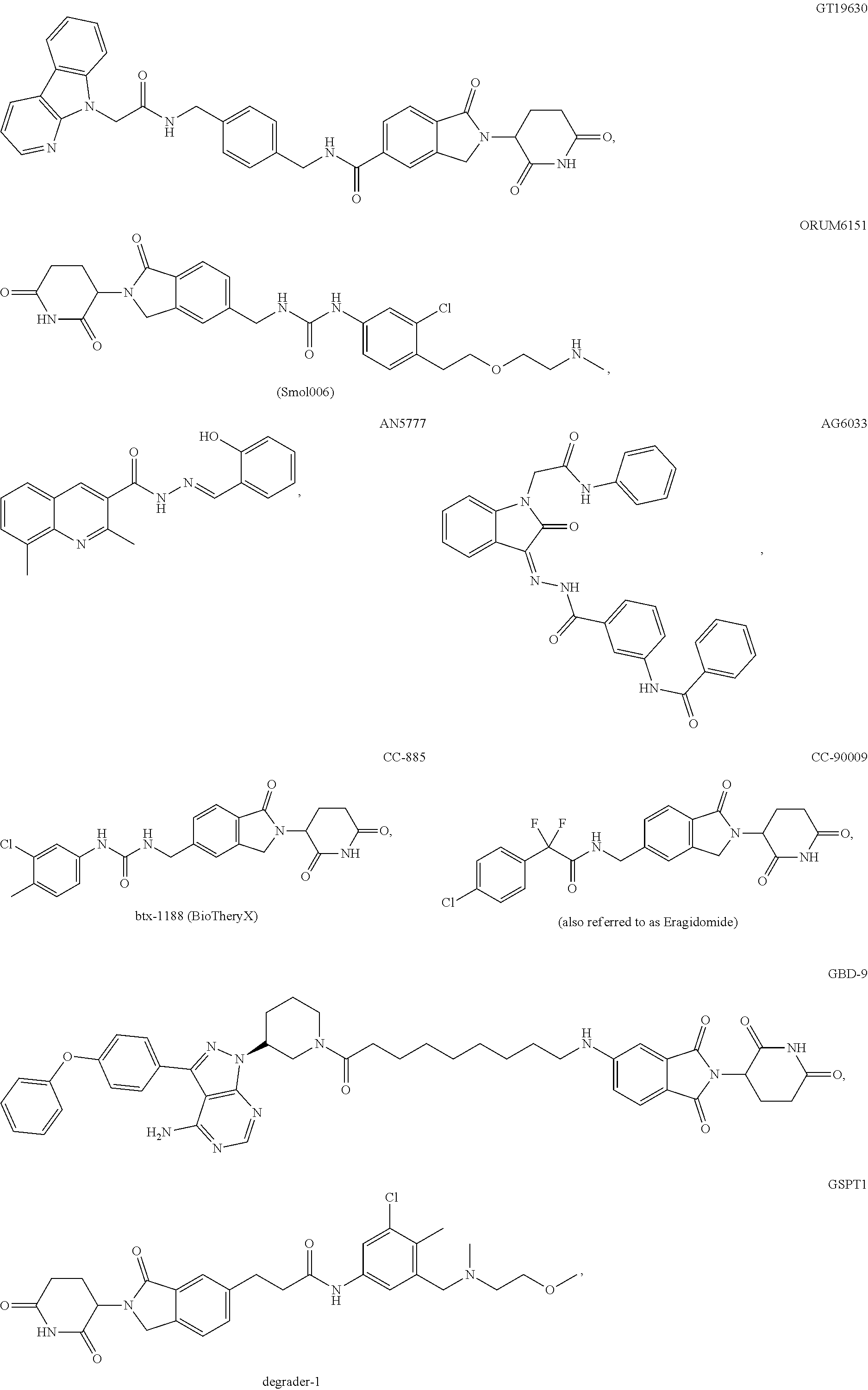

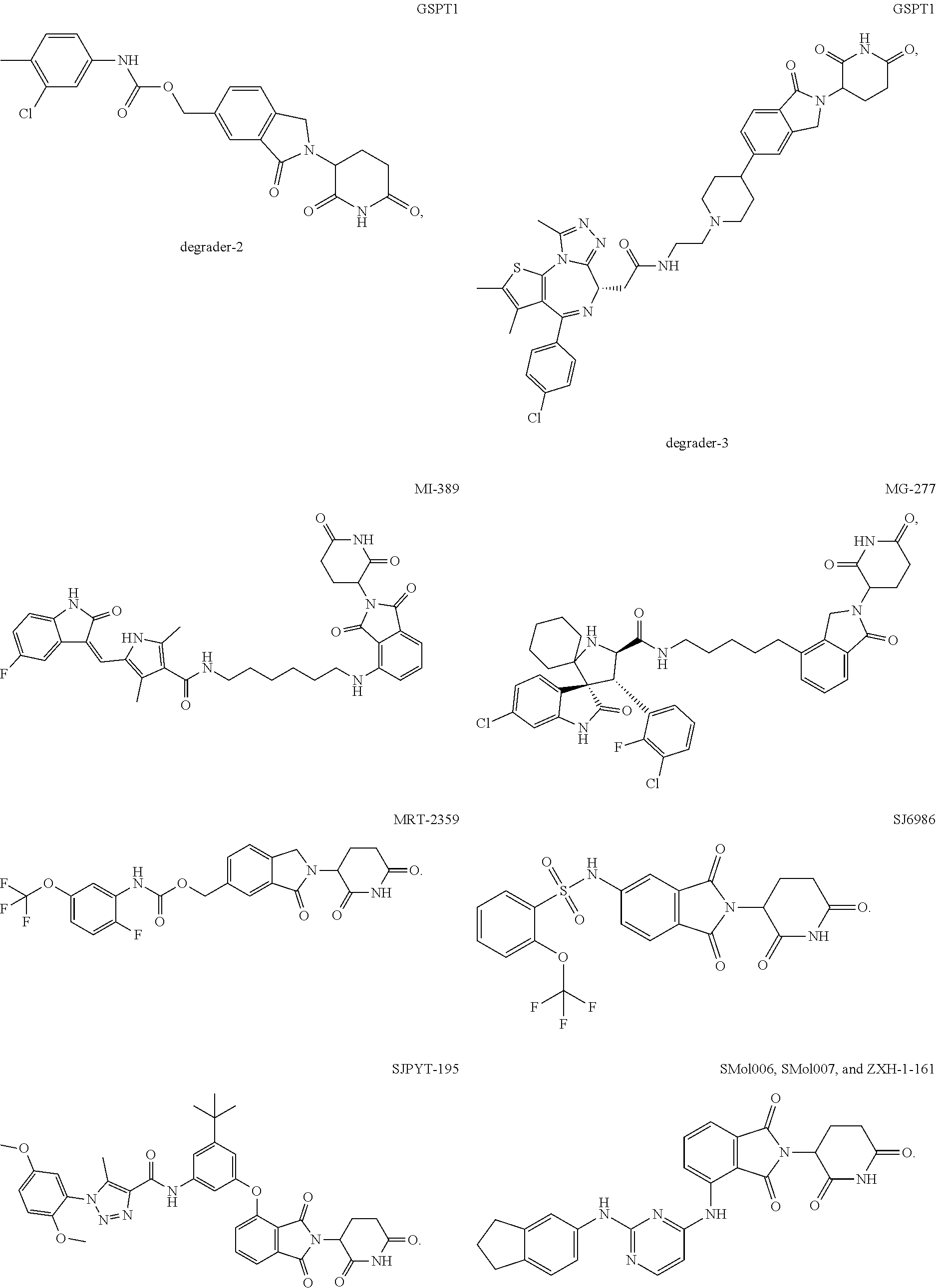

Embodiment 47 provides the method of embodiment 38, wherein the composition is formulated as a cream, a gel, a lotion, an ointment, a patch, a paste, a self-sticking membrane, or a spray.

Embodiment 48 provides a method of screening for therapeutic targets for regulating T cell function in a tissue of interest, comprising:
preparing a pool of genetically engineered T cells, wherein all or some of the genetically engineered T cells comprises one or more knocked-out or disrupted genes;
introducing the pool of genetically engineered T cells into a subject carrying the tissue of interest;
collecting T cells from a plurality of tissues including the tissue of interest;
detecting genes that are knocked-out or disrupted in the T cells,
wherein
a gene is determined to inhibit T cell function in the tissue of interest if the gene is knocked-out or disrupted at a higher rate in T cells in the tissue of interest than in other tissues, or
a gene is determined to enhance T cell function in the tissue of interest if the gene is knocked-out or disrupted at a lower rate in T cells in the tissue of interest than in other tissues.

Embodiment 49 provides the method of embodiment 48, wherein the genetically engineered T cells and the tissue of interest is originated from a first species, and wherein the subject is from a second species different from the first species.

Embodiment 50 provides the method of embodiment 49, wherein the first species is human, and the second species is a non-human species, optionally an immunodeficient non-human species.

Embodiment 51 provides the method of embodiment 48, wherein the method screens genes that cause or are associated with a T cell-mediated autoimmune disease in the tissue of interest, and wherein a gene is determined to cause or associate with the T cell-mediated autoimmune disease in the tissue of interest if the gene is knocked-out or disrupted at a higher rate in T cells found in the tissue of interest than in other tissues.

Embodiment 52 provides the method of embodiment 48, wherein the method screens genes that promote T cell mediated anti-cancer immunity, and wherein a gene is determined to promote T cell mediated anti-cancer immunity against a cancer tissue if the gene is knocked-out or disrupted at a lower rate in T cells found in the cancer tissue than in other tissues.

Embodiment 53 provides the method of embodiment 48, wherein preparing the pool of genetically engineered T cells comprises editing T cells with a CRISPR gene editing system comprising a gRNA library comprising gRNAs for targeting a plurality of genes in the T cells.

Embodiment 54 provides the method of embodiment 53, wherein the gRNA library is a genome-wide gRNA library.

Embodiment 55 provides the method of embodiment 48, further comprising confirming that the gene identified in the screening inhibits or enhances T cell function.

Embodiment 56 provides the method of embodiment 55, wherein confirming that the gene identified in the screening inhibits or enhances T cell function comprises:
introducing T cells into a subject carrying the tissue of interest;
isolating a plurality of tissues including the tissue of interest;
detecting a level of the gene identified in the screening,
wherein the gene is confirmed to inhibit T cell function in the tissue of interest if the level of the gene is reduced in the tissue of interest as compared to that in other tissues, and
   wherein the gene is confirmed to enhance T cell function in the tissue of interest if the level of the gene is increased in the tissue of interest as compared to that in other tissues.

### OTHER EMBODIMENTS

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of reducing skin-homing T cells or tissue-resident memory T cells (TRM) in a skin tissue, comprising contacting the skin-homing T cells or TRM with an effective amount of a composition that downregulates an expression level and/or an activity of GSPT1.

2. The method of claim 1, wherein the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

3. The method of claim 1, wherein the composition is delivered by a T cell-specific delivery method.

4. The method of claim 1, wherein the composition comprises a lipid nanoparticle (LNP) that is conjugated to an antibody targeting a T cell specific surface antigen.

5. The method of claim 4, wherein the T cell specific surface antigen is CD5.

6. The method of claim 1, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

7. The method of claim 1, wherein the composition comprises a molecular degrader that links GSPT1 to a ubiquitin ligase.

8. The method of claim 1, wherein the composition comprises a small molecule inhibitor of GSPT1, and wherein the small molecule inhibitor of GSPT1 comprises at least one selected form the group consisting of SP-3204, CDG0501,

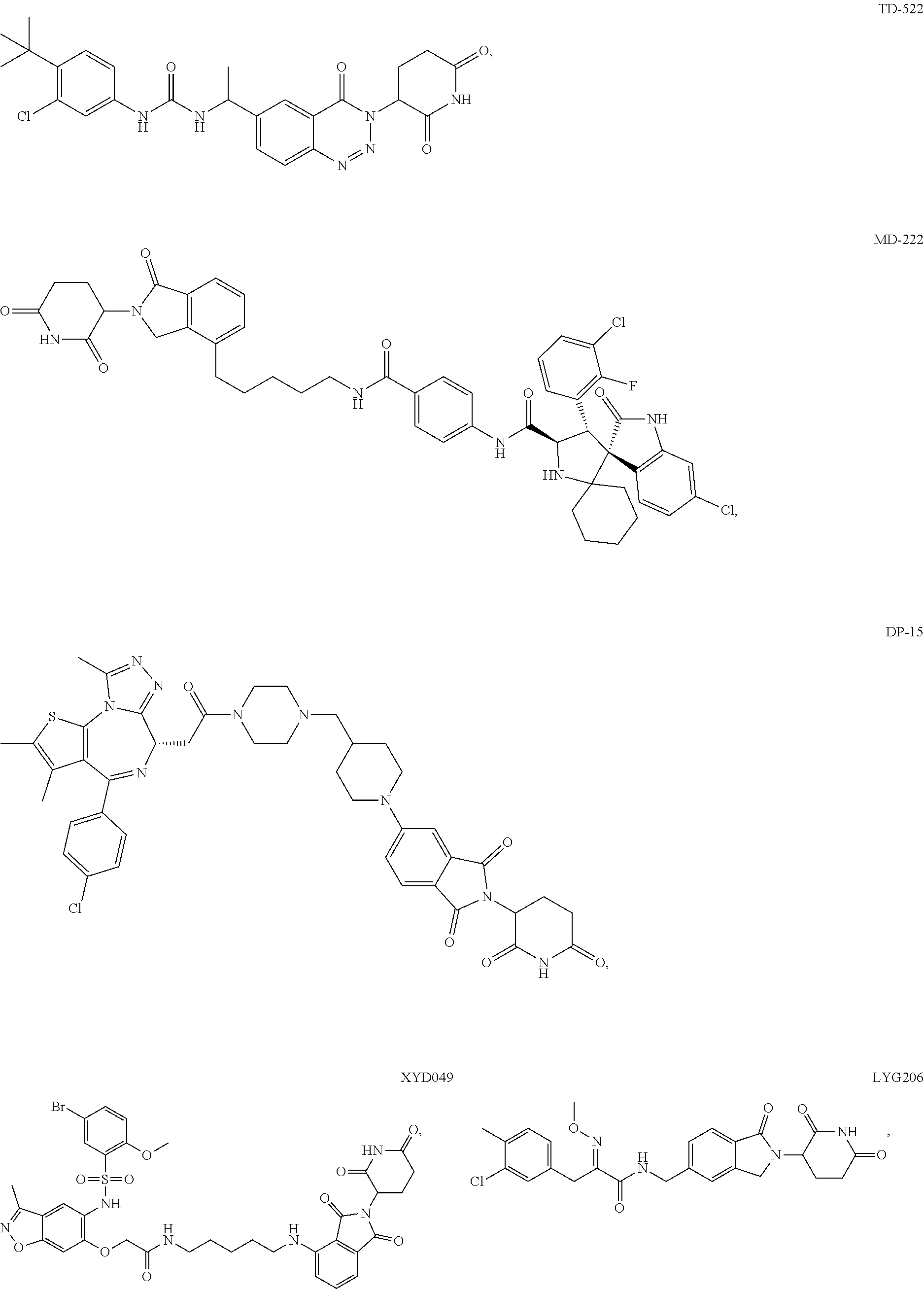 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O ; CO/N=C(\Cc1ccc(C)c(Cl)c1)C(=O)NCc1ccc2c(c1)CN(C1CCC(=O)NC1=O)C2=O]

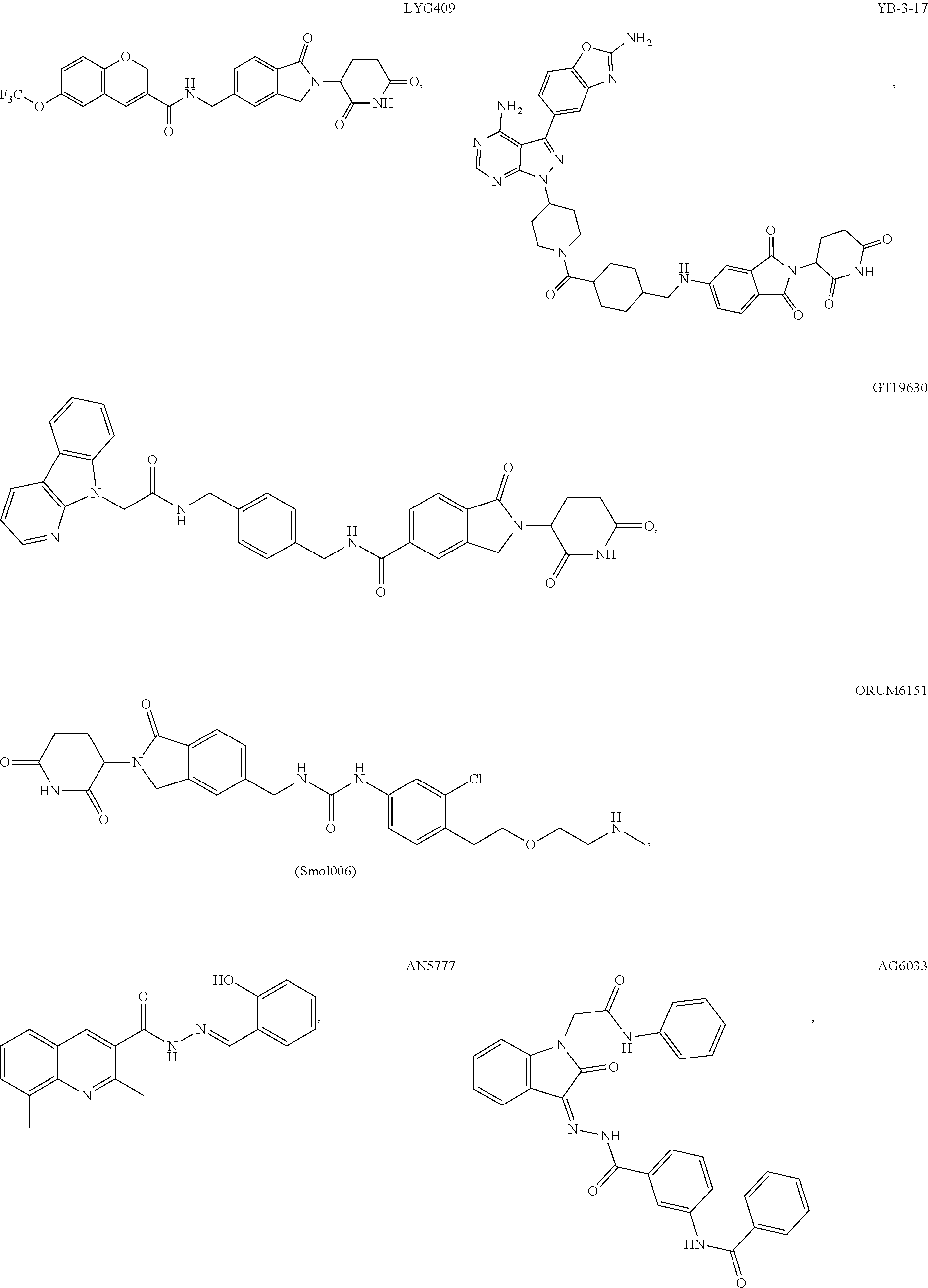 [SMILES: O=C1CCC(N2Cc3cc(CNC(=O)C4=Cc5cc(OC(F)(F)F)ccc5OC4)ccc3C2=O)C(=O)N1 ; Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl ; Cc1nc2c(C)cccc2cc1C(=O)N/N=C/c1ccccc1O ; O=C(CN1C(=O)/C(=N\NC(=O)c2cccc(NC(=O)c3ccccc3)c2)c2ccccc21)Nc1ccccc1]

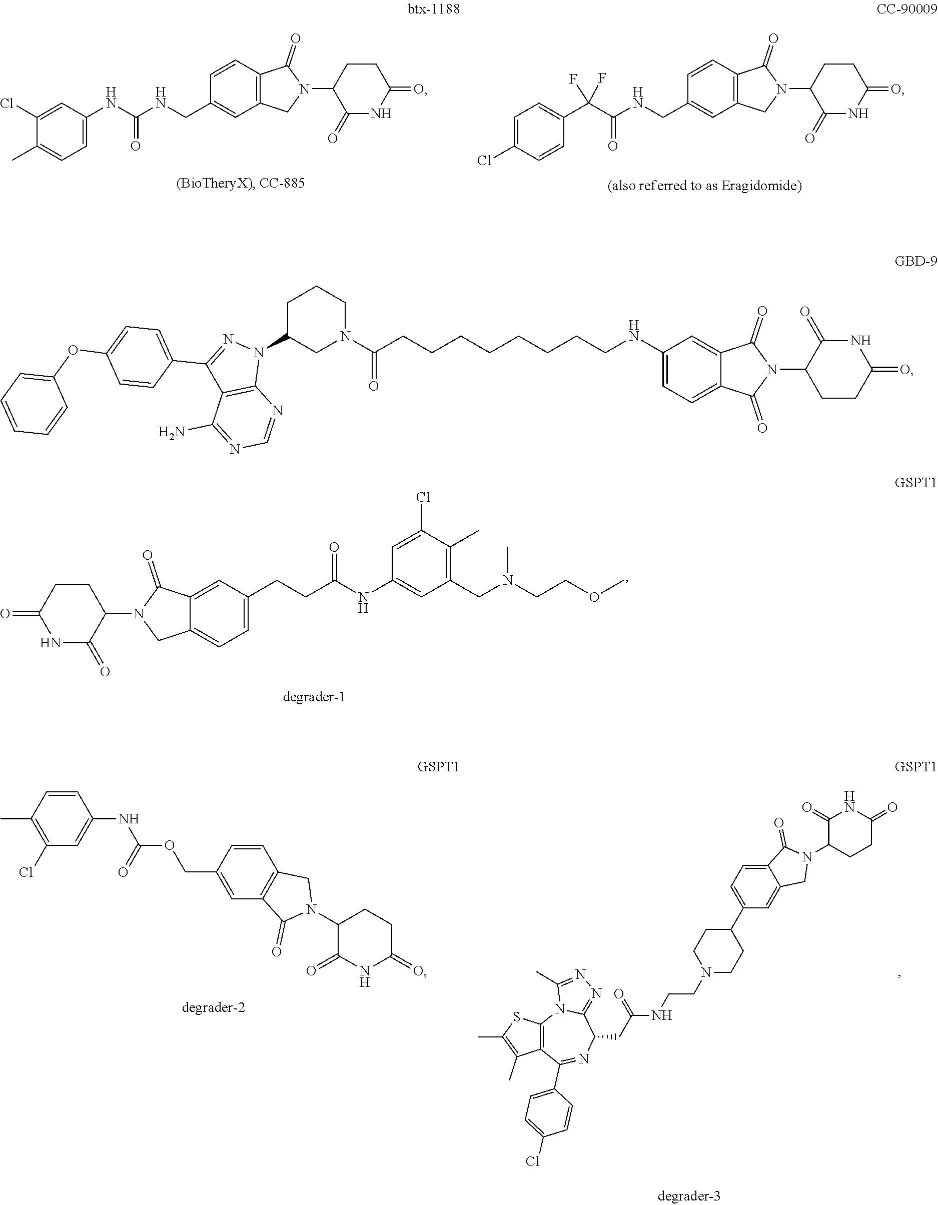

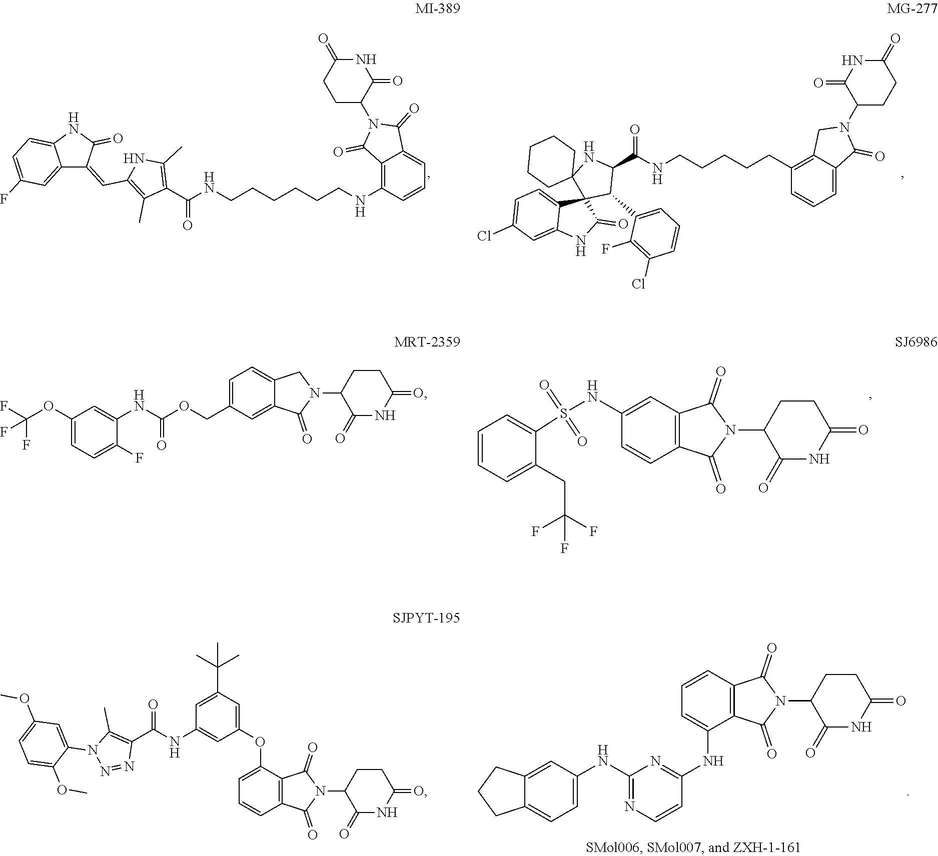

9. The method of claim 1, wherein administering an effective amount of the composition causes apoptosis in the TRM cells in the skin tissue.

10. The method of claim 1, wherein at least one of the following applies:
(i) the skin tissue is an isolated skin tissue; or
(ii) the skin tissue is in a subject.

11. The method of claim 1, wherein the skin tissue is in a subject, and wherein the subject is a mammal, optionally a human.

12. A method of selectively depleting T cells in the skin, comprising:
contacting T cells in the skin with a composition that downregulates an expression level and/or an activity of GSPT1.

13. The method of claim 12, wherein the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

14. The method of claim 12, wherein the composition is delivered by a T cell-specific delivery method.

15. The method of claim 12, wherein the composition comprises a lipid nanoparticle, wherein the lipid nanoparticle is conjugated to an antibody targeting a T cell specific surface antigen.

16. The method of claim 15, wherein the T cell specific surface antigen is CD5.

17. The method of claim 12, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

18. The method of claim 12, wherein the composition comprises a molecular degrader that links GSPT1 to a ubiquitin ligase.

19. The method of claim 12, wherein the composition comprises a small molecule inhibitor of GSPT1, and wherein the small molecule inhibitor of GSPT1 comprises at least one compound selected form the group consisting of SP-3204, CDG0501,

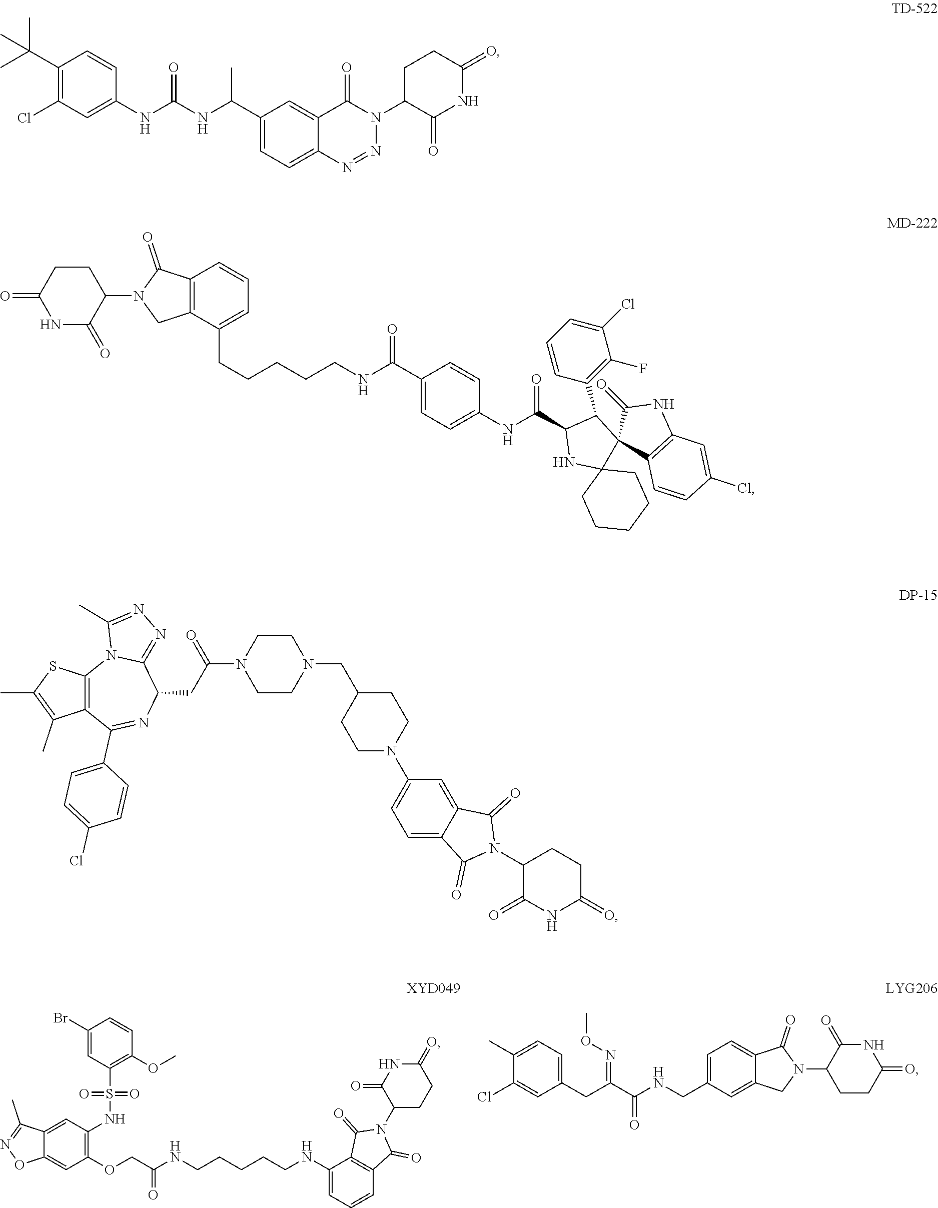 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O ; CO/N=C(\Cc1ccc(C)c(Cl)c1)C(=O)NCc1ccc2c(c1)CN(C1CCC(=O)NC1=O)C2=O]

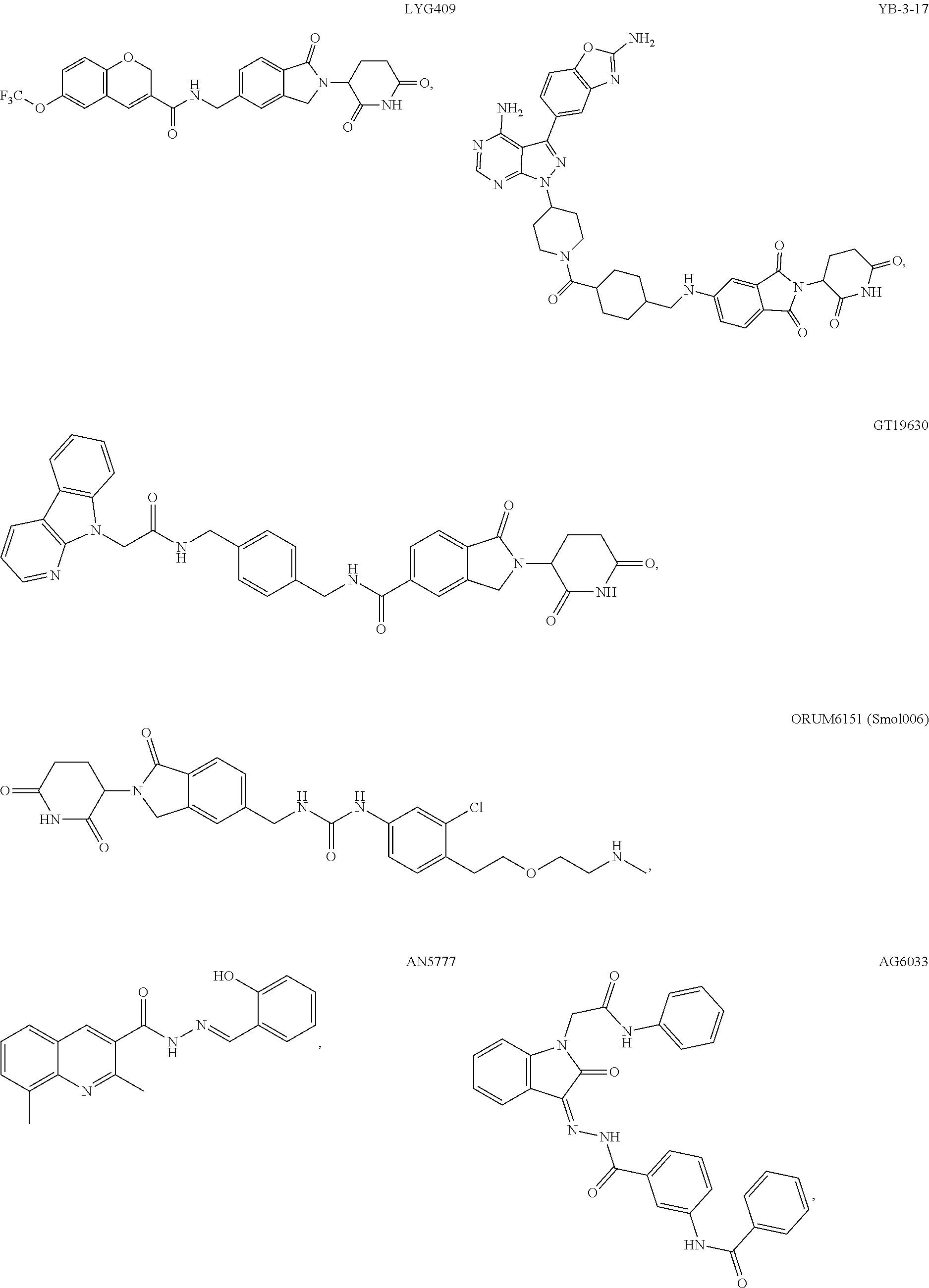 [SMILES: O=C1CCC(N2Cc3cc(CNC(=O)C4=Cc5cc(OC(F)(F)F)ccc5OC4)ccc3C2=O)C(=O)N1 ; Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl ; Cc1nc2c(C)cccc2cc1C(=O)N/N=C/c1ccccc1O ; O=C(CN1C(=O)/C(=N\NC(=O)c2cccc(NC(=O)c3ccccc3)c2)c2ccccc21)Nc1ccccc1]

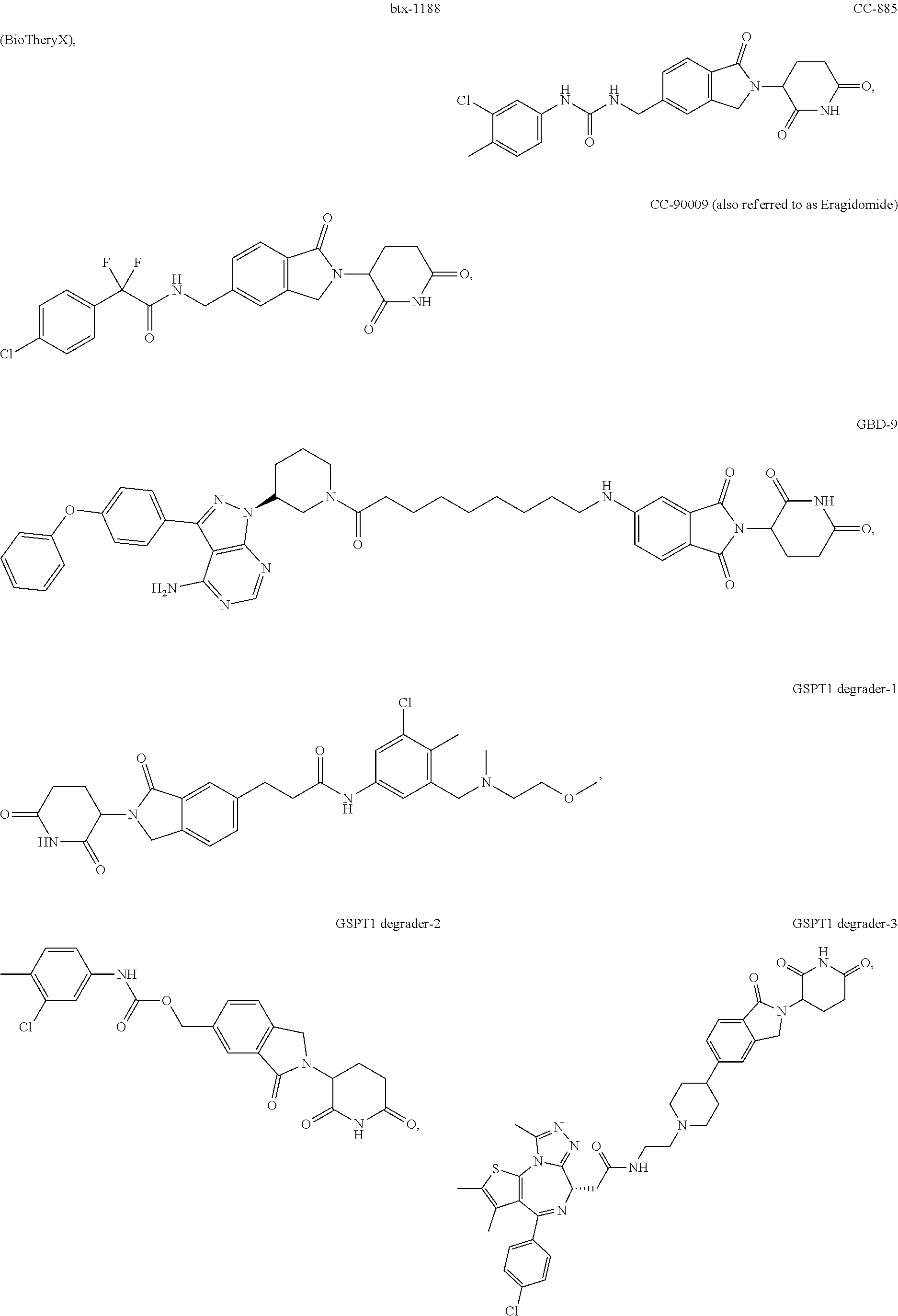

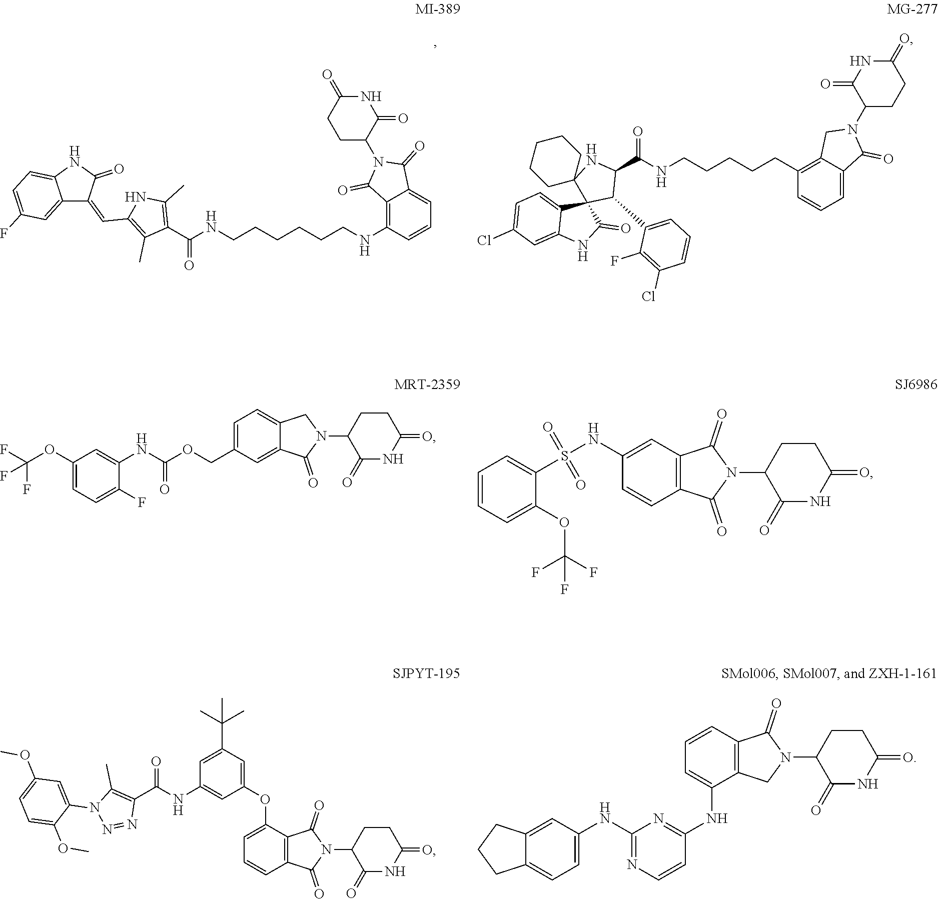 [SMILES: Cc1[nH]c(/C=C2\C(=O)Nc3ccc(F)cc32)c(C)c1C(=O)NCCCCCCNc1cccc2c1C(=O)N(C1CCC(=O)NC1=O)C2=O ; O=C1CCC(N2Cc3c(CCCCCNC(=O)[C@@H]4NC5(CCCCC5)[C@@]5(C(=O)Nc6cc(Cl)ccc65)[C@H]4c4cccc(Cl)c4F)cccc3C2=O)C(=O)N1 ; O=C1CCC(N2Cc3ccc(COC(=O)Nc4cc(OC(F)(F)F)ccc4F)cc3C2=O)C(=O)N1 ; O=C1CCC(N2C(=O)c3ccc(NS(=O)(=O)c4ccccc4OC(F)(F)F)cc3C2=O)C(=O)N1 ; COc1ccc(OC)c(-n2nnc(C(=O)Nc3cc(Oc4cccc5c4C(=O)N(C4CCC(=O)NC4=O)C5=O)cc(C(C)(C)C)c3)c2C)c1 ; O=C1CCC(N2Cc3c(Nc4ccnc(Nc5ccc6c(c5)CCC6)n4)cccc3C2=O)C(=O)N1]

20. The method of claim 12, wherein administering an effective amount of the compound causes apoptosis in the TRM cells in the skin tissue.

21. The method of any one of claim 12, wherein at least one of the following applies:
(i) the skin tissue is an isolated skin tissue; or
(ii) the skin tissue is in a subject.

22. The method of claim 12, wherein the skin tissue is in a subject, and wherein the subject is a mammal, optionally a human.

23. A method of treating, ameliorating and/or preventing an autoimmune or inflammatory disease or disorder in a skin in a subject in need thereof, comprising:
administering to the subject an effective amount of a composition that downregulates an expression level and/or an activity of GSPT1.

24. The method of claim 23, wherein the autoimmune or inflammatory disease or disorder in the skin is alopecia areata, graft-versus-host disease (GVHD), lichen planus, lupus erythematosus, scleroderma (optionally localized forms such as morphea), psoriasis and related conditions such as psoriatic arthritis, Stevens-Johnson syndrome, urticarial vasculitis, vitiligo, atopic dermatitis (eczema), or bullous pemphigoid.

25. The method of claim 23, wherein the composition comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

26. The method of claim 23, wherein the composition is delivered by a T cell-specific delivery method.

27. The method of claim 23, wherein the compound further comprises a lipid nanoparticle, wherein the lipid nanoparticle is conjugated to an antibody targeting a T cell specific surface antigen.

28. The method of claim 27, wherein the T cell specific surface antigen is CD5.

29. The method of claim 25, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

30. The method of claim 23, wherein the composition comprises a molecular degrader that links GSPT1 to a ubiquitin ligase.

31. The method of claim 23, wherein the composition comprises a small molecule inhibitor of GSPT1, and wherein the small molecule inhibitor of GSPT1 comprises at least one selected form the group consisting of SP-3204, CDG0501,

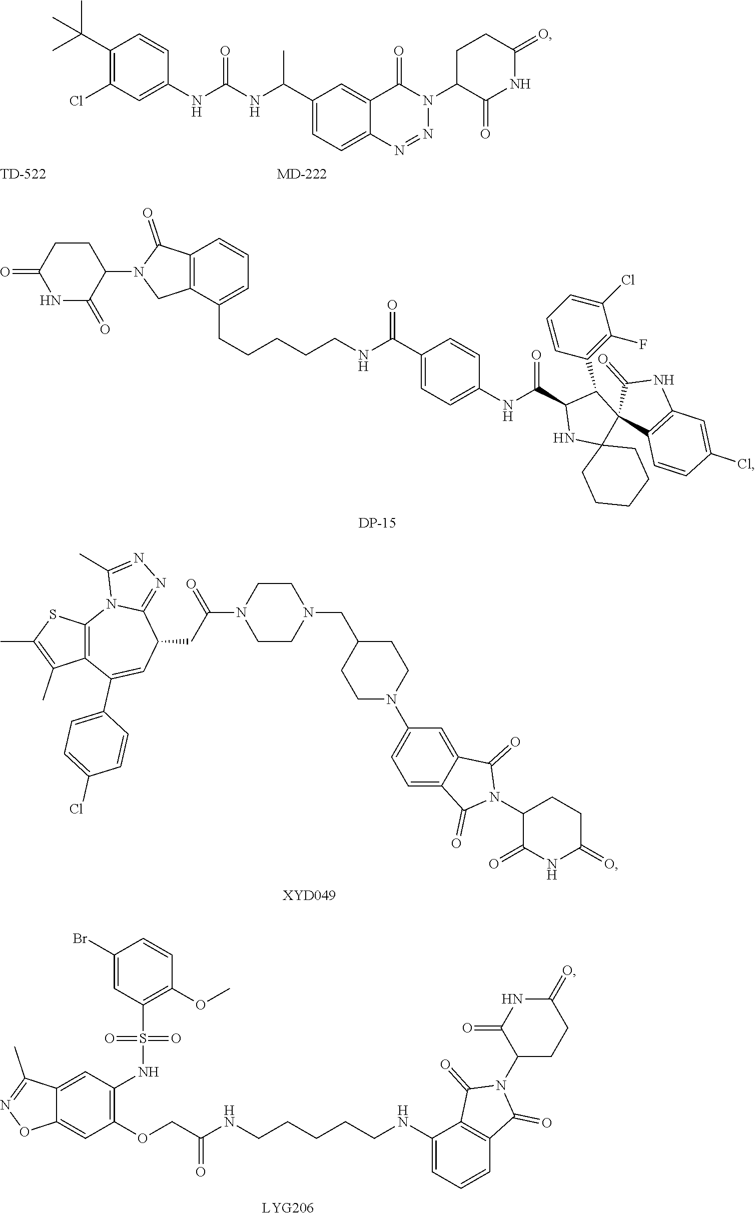

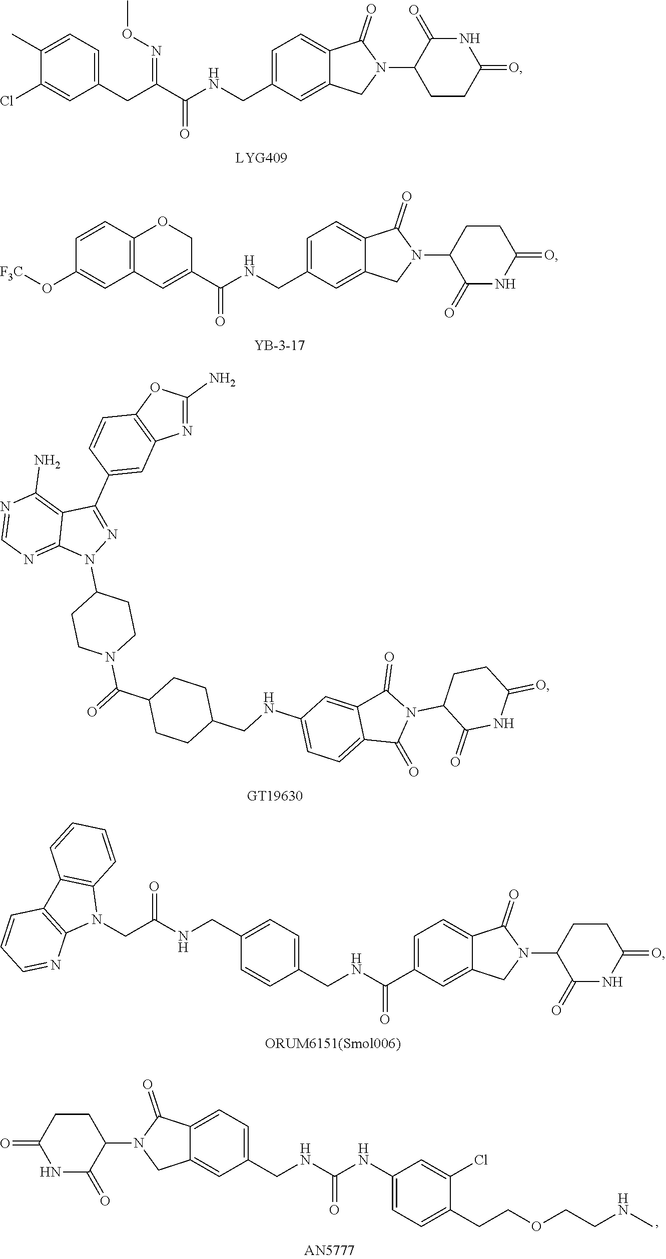 [SMILES: CO/N=C(\Cc1ccc(C)c(Cl)c1)C(=O)NCc1ccc2c(c1)CN(C1CCC(=O)NC1=O)C2=O ; O=C1CCC(N2Cc3cc(CNC(=O)C4=Cc5cc(OC(F)(F)F)ccc5OC4)ccc3C2=O)C(=O)N1 ; Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1 ; O=C(Cn1c2ccccc2c2cccnc21)NCc1ccc(CNC(=O)c2ccc3c(c2)CN(C2CCC(=O)NC2=O)C3=O)cc1 ; CNCCOCCc1ccc(NC(=O)NCc2ccc3c(c2)CN(C2CCC(=O)NC2=O)C3=O)cc1Cl]

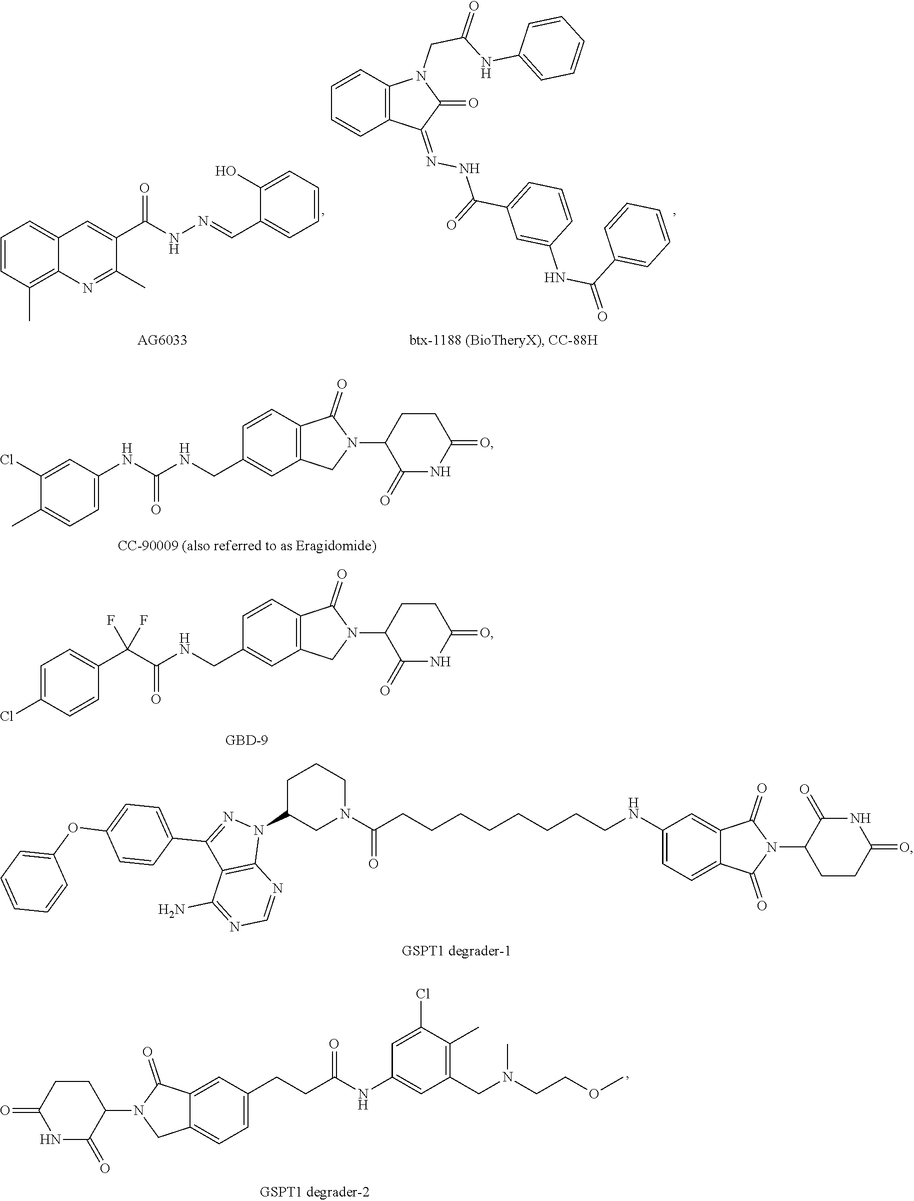

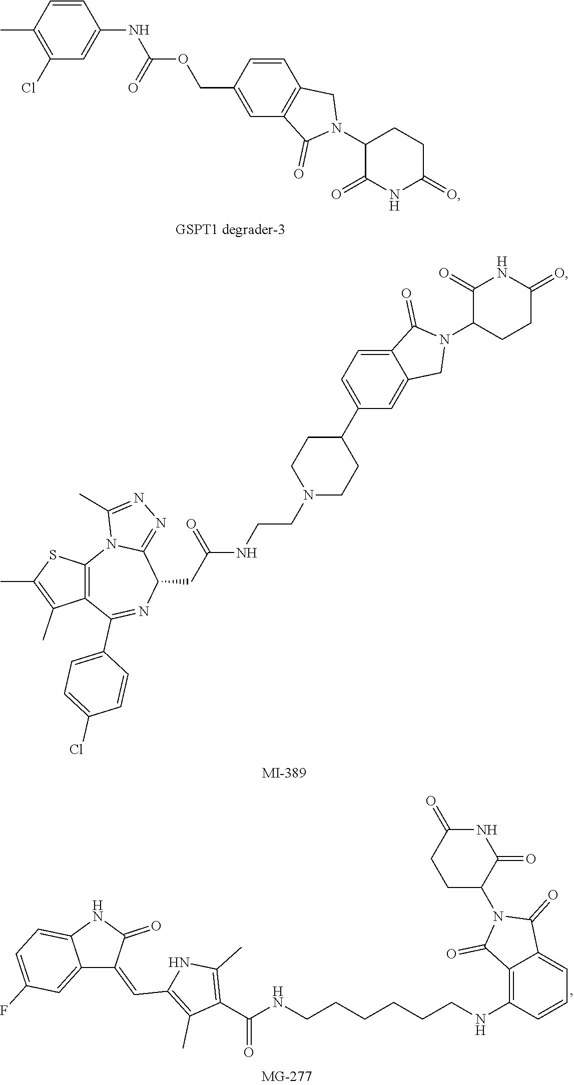 [SMILES: Cc1ccc(NC(=O)OCc2ccc3c(c2)C(=O)N(C2CCC(=O)NC2=O)C3)cc1Cl ; Cc1sc2c(c1C)C(c1ccc(Cl)cc1)=N[C@@H](CC(=O)NCCN1CCC(c3ccc4c(c3)CN(C3CCC(=O)NC3=O)C4=O)CC1)c1nnc(C)n1-2 ; Cc1[nH]c(/C=C2\C(=O)Nc3ccc(F)cc32)c(C)c1C(=O)NCCCCCCNc1cccc2c1C(=O)N(C1CCC(=O)NC1=O)C2=O]

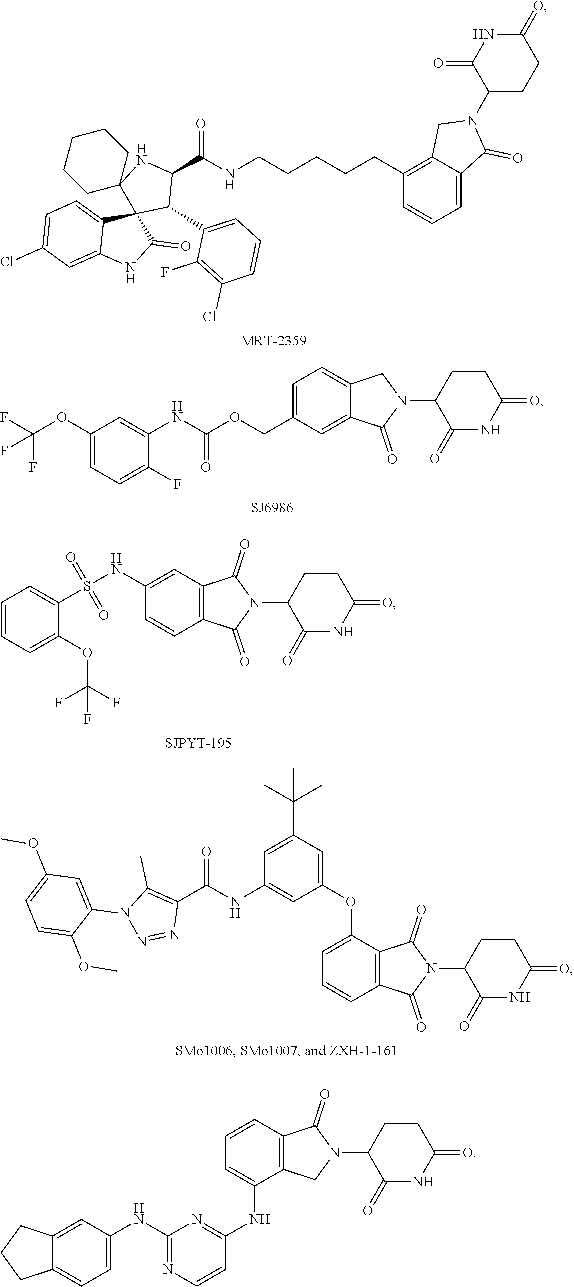 [SMILES: O=C1CCC(N2Cc3c(CCCCCNC(=O)[C@@H]4NC5(CCCCC5)[C@@]5(C(=O)Nc6cc(Cl)ccc65)[C@H]4c4cccc(Cl)c4F)cccc3C2=O)C(=O)N1 ; O=C1CCC(N2Cc3ccc(COC(=O)Nc4cc(OC(F)(F)F)ccc4F)cc3C2=O)C(=O)N1 ; O=C1CCC(N2C(=O)c3ccc(NS(=O)(=O)c4ccccc4OC(F)(F)F)cc3C2=O)C(=O)N1 ; COc1ccc(OC)c(-n2nnc(C(=O)Nc3cc(Oc4cccc5c4C(=O)N(C4CCC(=O)NC4=O)C5=O)cc(C(C)(C)C)c3)c2C)c1 ; O=C1CCC(N2Cc3c(Nc4ccnc(Nc5ccc6c(c5)CCC6)n4)cccc3C2=O)C(=O)N1]

32. The method of claim 23, wherein administering the composition causes apoptosis in skin-homing T cells.

33. The method of claim 23, wherein administering the composition causes apoptosis in the skin tissue resident memory T cells (TRMs).

34. The method of claim 23, wherein the composition is a pharmaceutical composition.

35. The method of claim 23, wherein the composition is administered topically, orally, or parentally.

36. The method of claim 23, wherein the subject is a mammal, optionally a human.

37. A pharmaceutical composition, comprising:
a compound that downregulates an expression level and/or an activity of GSPT1; and
a pharmaceutically acceptable carrier,
wherein the pharmaceutical composition is formulated as a topical formulation.

38. A method for treating, ameliorating, and/or preventing an autoimmune or inflammatory disease or disorder in a skin in a subject in need thereof, the method comprising administering an effective amount of the pharmaceutical composition comprising:
a compound that downregulates an expression level and/or an activity of GSPT1; and
a pharmaceutically acceptable carrier,
wherein the pharmaceutical composition is formulated as a topical formulation.

39. The method of claim 38, wherein the autoimmune or inflammatory disease or disorder is selected from the group consisting of alopecia areata, graft-versus-host disease (GVHD), lichen planus, lupus erythematosus, scleroderma, localized scleroderma such as morphea, psoriasis, psoriasis-related conditions such as psoriatic arthritis, Stevens-Johnson syndrome, urticarial vasculitis, vitiligo, atopic dermatitis (eczema), and bullous pemphigoid.

40. The method of claim 38, wherein the compound comprises at least one selected from the group consisting of:
a small molecule inhibitor of GSPT1,
a protein inhibitor of GSPT1,
a nucleic acid that downregulates the expression level and/or activity of GSPT1 by RNA interference, and/or an expression vector expressing the nucleic acid that downregulates the expression level and/or activity of GSPT1 by RNA interference,
a ribozyme that downregulates the expression level and/or activity of GSPT1, and/or an expression vector expressing the ribozyme,
an expression vector comprising an expression cassette, wherein the expression cassette expresses CRISPR components that downregulate the expression level and/or activity of GSPT1 by CRISPR knockout or CRISPR knockdown, and
a trans-dominant negative mutant protein of GSPT1, and/or an expression vector that expresses the trans-dominant negative mutant protein of GSPT1.

41. The method of claim 38, wherein the composition is formulated for a T cell-specific delivery method.

42. The method of claim 38, wherein composition comprises a lipid nanoparticle (LNP) conjugated to an antibody targeting a T cell specific surface antigen.

43. The method of claim 42, wherein the T cell specific surface antigen is CD5.

44. The method of claim 40, wherein the CRISPR components comprise one or more guide RNAs comprising a nucleic acid sequence selected from the group consisting of SEQ ID NO: 5, SEQ ID NO: 6, and a combination of SEQ ID NO: 5 and SEQ ID NO: 6.

45. The method of claim 40, wherein the compound comprises a molecular degrader that links GSPT1 to a ubiquitin ligase.

46. The method of claim 40, wherein the compound comprises a small molecule inhibitor of GSPT1, and wherein the small molecule inhibitor of GSPT1 comprises at least one selected form the group consisting of SP-3204, CDG0501,

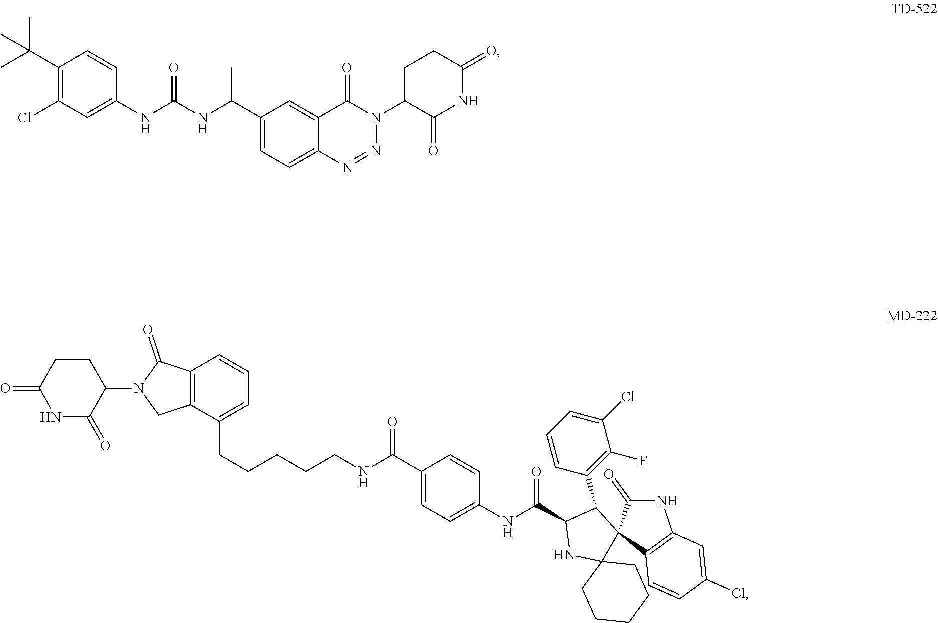 [SMILES: CC(NC(=O)Nc1ccc(C(C)(C)C)c(Cl)c1)c1ccc2nnn(C3CCC(=O)NC3=O)c(=O)c2c1 ; O=C1CCC(N2Cc3c(CCCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1]

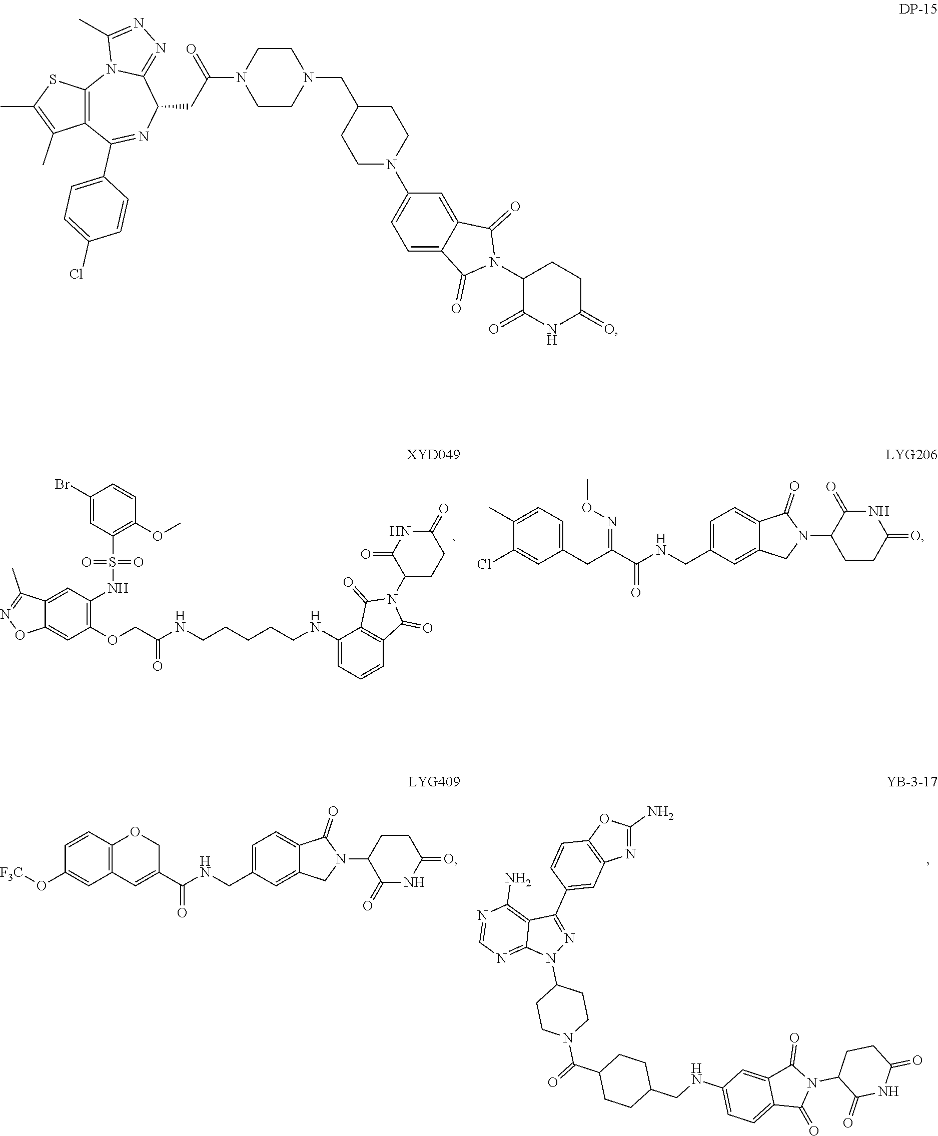 [SMILES: Cc1sc2c(c1C)C(c1ccc(Cl)cc1)=N[C@@H](CC(=O)N1CCN(CC3CCN(c4ccc5c(c4)C(=O)N(C4CCC(=O)NC4=O)C5=O)CC3)CC1)c1nnc(C)n1-2 ; COc1ccc(Br)cc1S(=O)(=O)Nc1cc2c(C)noc2cc1OCC(=O)NCCCCCNc1cccc2c1C(=O)N(C1CCC(=O)NC1=O)C2=O ; CO/N=C(\Cc1ccc(C)c(Cl)c1)C(=O)NCc1ccc2c(c1)CN(C1CCC(=O)NC1=O)C2=O ; O=C1CCC(N2Cc3cc(CNC(=O)C4=Cc5cc(OC(F)(F)F)ccc5OC4)ccc3C2=O)C(=O)N1 ; Nc1nc2cc(-c3nn(C4CCN(C(=O)C5CCC(CNc6ccc7c(c6)C(=O)N(C6CCC(=O)NC6=O)C7=O)CC5)CC4)c4ncnc(N)c34)ccc2o1]

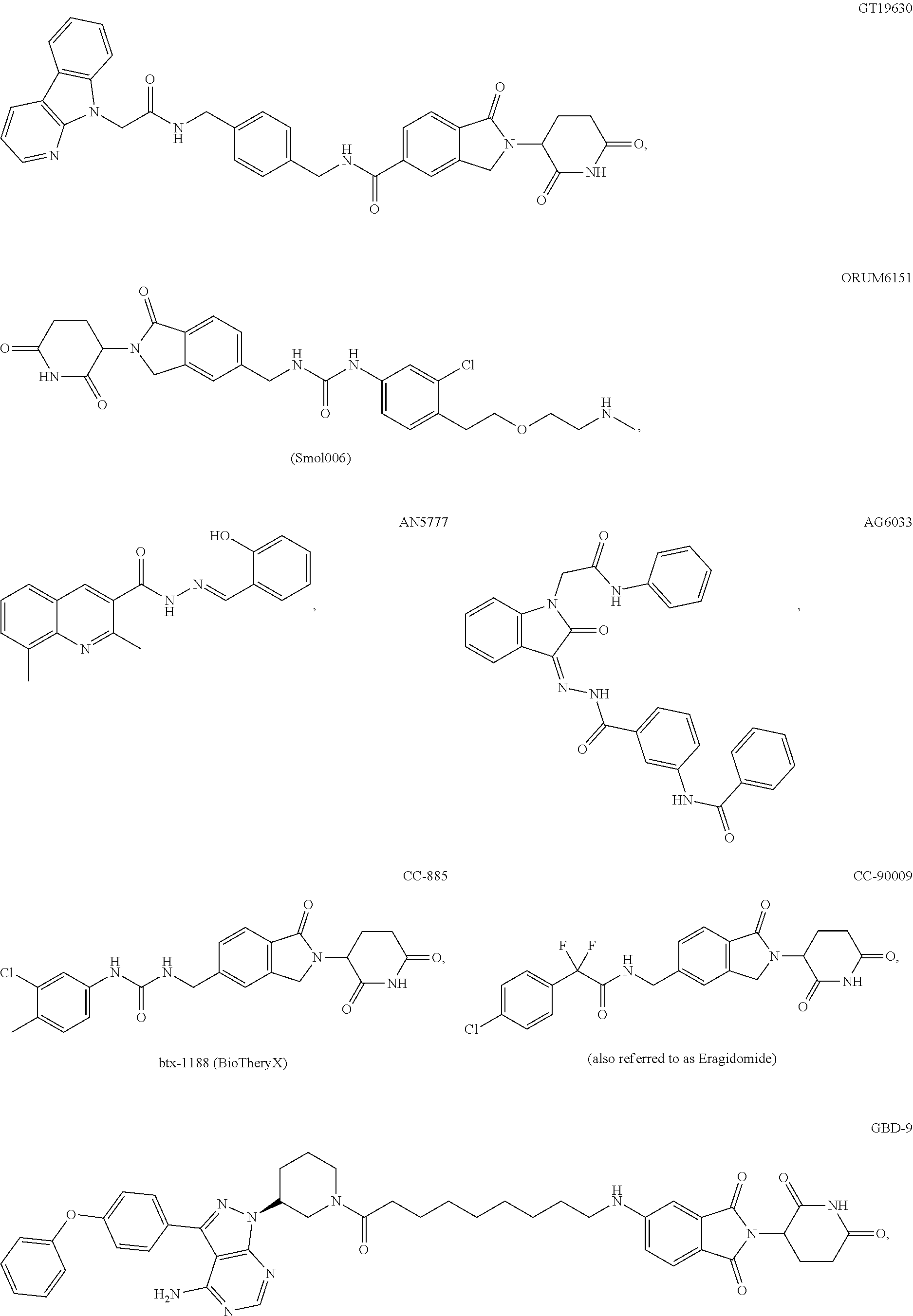

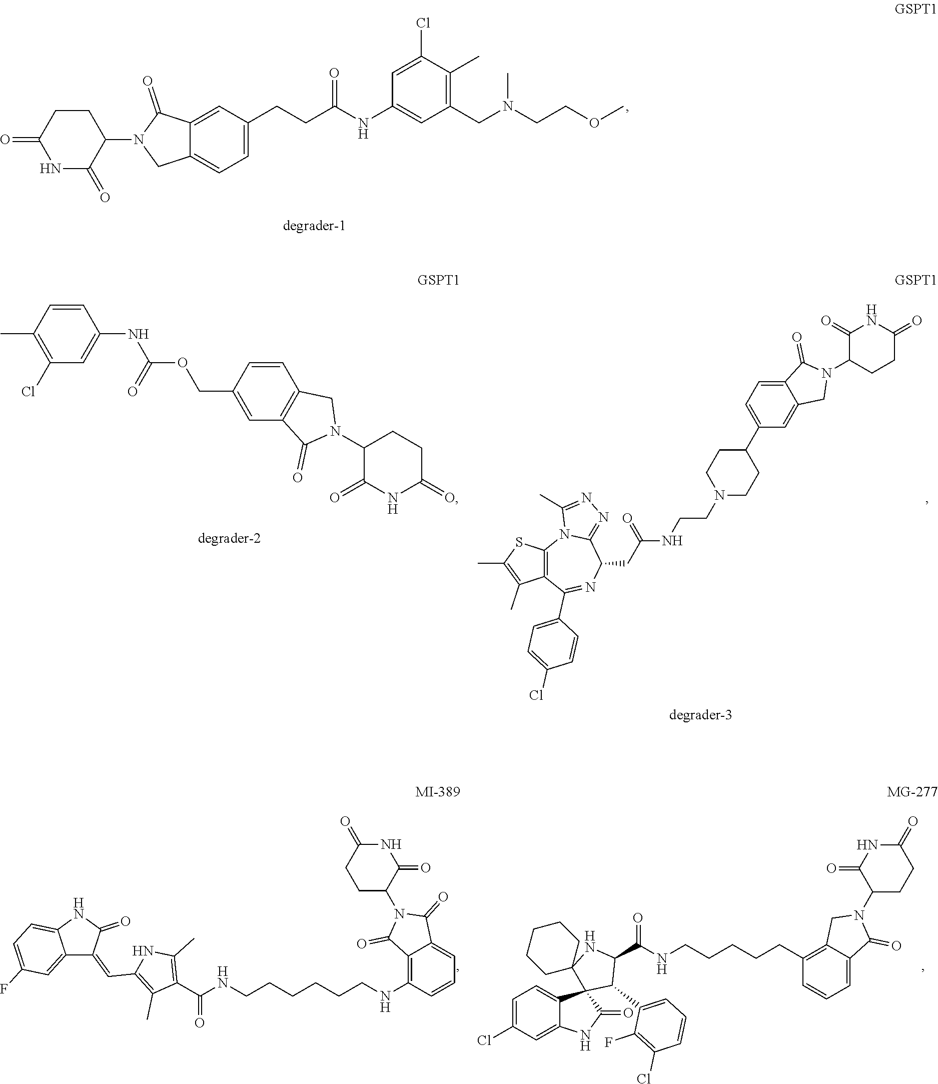

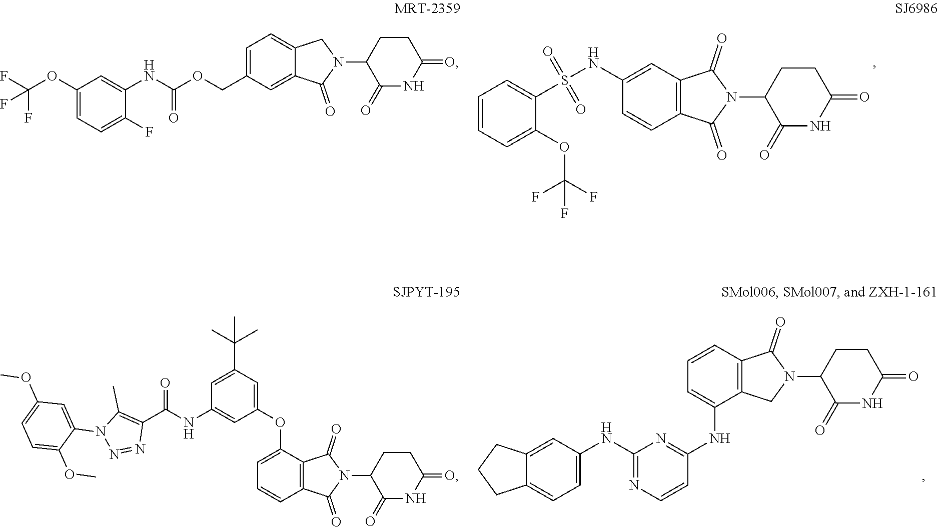

47. The method of claim 38, wherein the composition is formulated as a cream, a gel, a lotion, an ointment, a patch, a paste, a self-sticking membrane, or a spray.

48. A method of screening for therapeutic targets for regulating T cell function in a tissue of interest, comprising:
preparing a pool of genetically engineered T cells, wherein all or some of the genetically engineered T cells comprises one or more knocked-out or disrupted genes;
introducing the pool of genetically engineered T cells into a subject carrying the tissue of interest;
collecting T cells from a plurality of tissues including the tissue of interest;
detecting genes that are knocked-out or disrupted in the T cells,
wherein
a gene is determined to inhibit T cell function in the tissue of interest if the gene is knocked-out or disrupted at a higher rate in T cells in the tissue of interest than in other tissues, or
a gene is determined to enhance T cell function in the tissue of interest if the gene is knocked-out or disrupted at a lower rate in T cells in the tissue of interest than in other tissues.

49. The method of claim 48, wherein the genetically engineered T cells and the tissue of interest is originated from a first species, and wherein the subject is from a second species different from the first species.

50. The method of claim 49, wherein the first species is human, and the second species is a non-human species, optionally an immunodeficient non-human species.

51. The method of claim 48, wherein the method screens genes that cause or are associated with a T cell-mediated autoimmune disease in the tissue of interest, and wherein a gene is determined to cause or associate with the T cell-mediated autoimmune disease in the tissue of interest if the gene is knocked-out or disrupted at a higher rate in T cells found in the tissue of interest than in other tissues.

52. The method of claim 48, wherein the method screens genes that promote T cell mediated anti-cancer immunity, and wherein a gene is determined to promote T cell mediated anti-cancer immunity against a cancer tissue if the gene is knocked-out or disrupted at a lower rate in T cells found in the cancer tissue than in other tissues.

53. The method of claim 48, wherein preparing the pool of genetically engineered T cells comprises editing T cells with a CRISPR gene editing system comprising a gRNA library comprising gRNAs for targeting a plurality of genes in the T cells.

54. The method of claim 53, wherein the gRNA library is a genome-wide gRNA library.

55. The method of claim 48, further comprising confirming that the gene identified in the screening inhibits or enhances T cell function.

56. The method of claim 55, wherein confirming that the gene identified in the screening inhibits or enhances T cell function comprises:
introducing T cells into a subject carrying the tissue of interest;
isolating a plurality of tissues including the tissue of interest;
detecting a level of the gene identified in the screening,
wherein the gene is confirmed to inhibit T cell function in the tissue of interest if the level of the gene is reduced in the tissue of interest as compared to that in other tissues, and
wherein the gene is confirmed to enhance T cell function in the tissue of interest if the level of the gene is increased in the tissue of interest as compared to that in other tissues.


SEQUENCE LISTING (9 sequences)

SEQ ID NO: 1 - DNA, 18 bp, synthetic construct
  source 1..18: mol_type = other DNA
ttcaggtatttgactgga

SEQ ID NO: 2 - protein, 4 aa, synthetic construct
YLTG

SEQ ID NO: 3 - DNA, 18 bp, synthetic construct
  source 1..18: mol_type = other DNA
ttcgggtgtttgactgga

SEQ ID NO: 4 - DNA, 18 bp, synthetic construct
  source 1..18: mol_type = other DNA
ttcaggtatttgactgga

SEQ ID NO: 5 - DNA, 20 bp, synthetic construct
  source 1..20: mol_type = other DNA
tttcaggtatttgactggaa

SEQ ID NO: 6 - DNA, 20 bp, synthetic construct
  source 1..20: mol_type = other DNA
ttatttcaggtacttgtctt

SEQ ID NO: 7 - DNA, 20 bp, synthetic construct
  source 1..20: mol_type = other DNA
ccagcgggagaacctcagcg

SEQ ID NO: 8 - DNA, 20 bp, synthetic construct
  source 1..20: mol_type = other DNA
aatcccaaaacctaagtctg

SEQ ID NO: 9 - DNA, 20 bp, synthetic construct
  source 1..20: mol_type = other DNA
aatgtctccagaagaatcat